SEQUENCE LISTING (190 sequences)

SEQ ID NO: 1-4 - skipped (4 entries)

SEQ ID NO: 5 - protein, 5 aa, synthetic construct
  REGION 1..5: note = Description of Artificial Sequence: Syntheticpeptide
QLVKS

SEQ ID NO: 6 - protein, 12 aa, synthetic construct
  REGION 1..12: note = Description of Artificial Sequence: Syntheticpeptide
SGTPHEVGVYTL

SEQ ID NO: 7 - protein, 12 aa, synthetic construct
  REGION 1..12: note = Description of Artificial Sequence: Syntheticpeptide
SGAIRCHDEFWF

SEQ ID NO: 8 - DNA, 28 bp, synthetic construct
  misc_feature 1..28: note = Description of Artificial Sequence: Syntheticoligonucleotide
  source 1..28: mol_type = other DNA
ttgctcttgaagtccatagacctcatgt

SEQ ID NO: 9 - DNA, 28 bp, synthetic construct
  misc_feature 1..28: note = Description of Artificial Sequence: Syntheticoligonucleotide
  source 1..28: mol_type = other DNA
gtgctgtggcctggagcaacaaatctga

SEQ ID NO: 10 - DNA, 28 bp, synthetic construct
  misc_feature 1..28: note = Description of Artificial Sequence: Syntheticoligonucleotide
  source 1..28: mol_type = other DNA
ctgttgctcttgaagtccatagacctca

SEQ ID NO: 11 - DNA, 28 bp, synthetic construct
  misc_feature 1..28: note = Description of Artificial Sequence: Syntheticoligonucleotide
  source 1..28: mol_type = other DNA
ctgtggcctggagcaacaaatctgactt

SEQ ID NO: 12 - DNA, 22 bp, synthetic construct
  misc_feature 1..22: note = Description of Artificial Sequence: Syntheticoligonucleotide
  source 1..22: mol_type = other DNA
aggattcggaacccaatcactg

SEQ ID NO: 13 - DNA, 28 bp, synthetic construct
  misc_feature 1..28: note = Description of Artificial Sequence: Syntheticoligonucleotide
  source 1..28: mol_type = other DNA
ctcctgaaagtggccgggtttaatctgc

SEQ ID NO: 14 - DNA, 28 bp, synthetic construct
  misc_feature 1..28: note = Description of Artificial Sequence: Syntheticoligonucleotide
  source 1..28: mol_type = other DNA
aggattcggaacccaatcactgacaggt

SEQ ID NO: 15 - DNA, 28 bp, synthetic construct
  misc_feature 1..28: note = Description of Artificial Sequence: Syntheticoligonucleotide
  source 1..28: mol_type = other DNA
tcaagctggtcgagaaaagctttgaaac

SEQ ID NO: 16 - DNA, 28 bp, synthetic construct
  misc_feature 1..28: note = Description of Artificial Sequence: Syntheticoligonucleotide
  source 1..28: mol_type = other DNA
aacaggtaagacaggggtctagcctggg

SEQ ID NO: 17 - DNA, 28 bp, synthetic construct
  misc_feature 1..28: note = Description of Artificial Sequence: Syntheticoligonucleotide
  source 1..28: mol_type = other DNA
ctgtgctagacatgaggtctatggactt

SEQ ID NO: 18 - DNA, 28 bp, synthetic construct
  misc_feature 1..28: note = Description of Artificial Sequence: Syntheticoligonucleotide
  source 1..28: mol_type = other DNA
ttcaagagcaacagtgctgtggcctgga

SEQ ID NO: 19 - DNA, 28 bp, synthetic construct
  misc_feature 1..28: note = Description of Artificial Sequence: Syntheticoligonucleotide
  source 1..28: mol_type = other DNA
cctgtcagtgattgggttccgaatcctc

SEQ ID NO: 20 - DNA, 28 bp, synthetic construct
  misc_feature 1..28: note = Description of Artificial Sequence: Syntheticoligonucleotide
  source 1..28: mol_type = other DNA
tcctcctgaaagtggccgggtttaatct

SEQ ID NO: 21 - DNA, 23 bp, synthetic construct
  misc_feature 1..23: note = Description of Artificial Sequence: Syntheticoligonucleotide
  source 1..23: mol_type = other DNA
ctgtgctagacatgaggtctatg

SEQ ID NO: 22 - protein, 7 aa, synthetic construct
  REGION 1..7: note = Description of Artificial Sequence: Syntheticpeptide
DRSNLSR

SEQ ID NO: 23 - protein, 7 aa, synthetic construct
  REGION 1..7: note = Description of Artificial Sequence: Syntheticpeptide
QKVTLAA

SEQ ID NO: 24 - protein, 7 aa, synthetic construct
  REGION 1..7: note = Description of Artificial Sequence: Syntheticpeptide
DRSALSR

SEQ ID NO: 25 - protein, 7 aa, synthetic construct
  REGION 1..7: note = Description of Artificial Sequence: Syntheticpeptide
TSGNLTR

SEQ ID NO: 26 - protein, 7 aa, synthetic construct
  REGION 1..7: note = Description of Artificial Sequence: Syntheticpeptide
YRSSLKE

SEQ ID NO: 27 - protein, 7 aa, synthetic construct
  REGION 1..7: note = Description of Artificial Sequence: Syntheticpeptide
QQNVLIN

SEQ ID NO: 28 - protein, 7 aa, synthetic construct
  REGION 1..7: note = Description of Artificial Sequence: Syntheticpeptide
QNATRTK

SEQ ID NO: 29 - protein, 7 aa, synthetic construct
  REGION 1..7: note = Description of Artificial Sequence: Syntheticpeptide
QSGHLAR

SEQ ID NO: 30 - protein, 7 aa, synthetic construct
  REGION 1..7: note = Description of Artificial Sequence: Syntheticpeptide
NRYDLMT

SEQ ID NO: 31 - protein, 7 aa, synthetic construct
  REGION 1..7: note = Description of Artificial Sequence: Syntheticpeptide
RSDSLLR

SEQ ID NO: 32 - protein, 7 aa, synthetic construct
  REGION 1..7: note = Description of Artificial Sequence: Syntheticpeptide
QSSDLTR

SEQ ID NO: 33 - protein, 7 aa, synthetic construct
  REGION 1..7: note = Description of Artificial Sequence: Syntheticpeptide
DRSALAR

SEQ ID NO: 34 - protein, 7 aa, synthetic construct
  REGION 1..7: note = Description of Artificial Sequence: Syntheticpeptide
QSGNLAR

SEQ ID NO: 35 - protein, 7 aa, synthetic construct
  REGION 1..7: note = Description of Artificial Sequence: Syntheticpeptide
HRSTLQG

SEQ ID NO: 36 - protein, 7 aa, synthetic construct
  REGION 1..7: note = Description of Artificial Sequence: Syntheticpeptide
QSGDLTR

SEQ ID NO: 37 - protein, 7 aa, synthetic construct
  REGION 1..7: note = Description of Artificial Sequence: Syntheticpeptide
TSGSLTR

SEQ ID NO: 38 - protein, 7 aa, synthetic construct
  REGION 1..7: note = Description of Artificial Sequence: Syntheticpeptide
QHQVLVR

SEQ ID NO: 39 - protein, 7 aa, synthetic construct
  REGION 1..7: note = Description of Artificial Sequence: Syntheticpeptide
QSGHLSR

SEQ ID NO: 40 - protein, 7 aa, synthetic construct
  REGION 1..7: note = Description of Artificial Sequence: Syntheticpeptide
DRSDLSR

SEQ ID NO: 41 - protein, 7 aa, synthetic construct
  REGION 1..7: note = Description of Artificial Sequence: Syntheticpeptide
RSDALAR

SEQ ID NO: 42 - protein, 7 aa, synthetic construct
  REGION 1..7: note = Description of Artificial Sequence: Syntheticpeptide
DQSNLRA

SEQ ID NO: 43 - protein, 7 aa, synthetic construct
  REGION 1..7: note = Description of Artificial Sequence: Syntheticpeptide
TSSNRKT

SEQ ID NO: 44 - protein, 7 aa, synthetic construct
  REGION 1..7: note = Description of Artificial Sequence: Syntheticpeptide
DSSTRKT

SEQ ID NO: 45 - protein, 7 aa, synthetic construct
  REGION 1..7: note = Description of Artificial Sequence: Syntheticpeptide
RSDDLSE

SEQ ID NO: 46 - protein, 7 aa, synthetic construct
  REGION 1..7: note = Description of Artificial Sequence: Syntheticpeptide
TNSNRKR

SEQ ID NO: 47 - protein, 7 aa, synthetic construct
  REGION 1..7: note = Description of Artificial Sequence: Syntheticpeptide
RSDHLST

SEQ ID NO: 48 - protein, 7 aa, synthetic construct
  REGION 1..7: note = Description of Artificial Sequence: Syntheticpeptide
DRSHLAR

SEQ ID NO: 49 - protein, 7 aa, synthetic construct
  REGION 1..7: note = Description of Artificial Sequence: Syntheticpeptide
LKQHLNE

SEQ ID NO: 50 - protein, 7 aa, synthetic construct
  REGION 1..7: note = Description of Artificial Sequence: Syntheticpeptide
HRTSLTD

SEQ ID NO: 51 - protein, 7 aa, synthetic construct
  REGION 1..7: note = Description of Artificial Sequence: Syntheticpeptide
LQQTLAD

SEQ ID NO: 52 - protein, 7 aa, synthetic construct
  REGION 1..7: note = Description of Artificial Sequence: Syntheticpeptide
RREDLIT

SEQ ID NO: 53 - protein, 7 aa, synthetic construct
  REGION 1..7: note = Description of Artificial Sequence: Syntheticpeptide
TSSNLSR

SEQ ID NO: 54 - protein, 7 aa, synthetic construct
  REGION 1..7: note = Description of Artificial Sequence: Syntheticpeptide
HNSSLKD

SEQ ID NO: 55 - protein, 7 aa, synthetic construct
  REGION 1..7: note = Description of Artificial Sequence: Syntheticpeptide
LNHHLQQ

SEQ ID NO: 56 - protein, 7 aa, synthetic construct
  REGION 1..7: note = Description of Artificial Sequence: Syntheticpeptide
HKTSLKD

SEQ ID NO: 57 - protein, 7 aa, synthetic construct
  REGION 1..7: note = Description of Artificial Sequence: Syntheticpeptide
QSSDLSR

SEQ ID NO: 58 - protein, 7 aa, synthetic construct
  REGION 1..7: note = Description of Artificial Sequence: Syntheticpeptide
QSGNRTT

SEQ ID NO: 59 - protein, 7 aa, synthetic construct
  REGION 1..7: note = Description of Artificial Sequence: Syntheticpeptide
RSANLAR

SEQ ID NO: 60 - protein, 7 aa, synthetic construct
  REGION 1..7: note = Description of Artificial Sequence: Syntheticpeptide
RSDVLSE

SEQ ID NO: 61 - protein, 7 aa, synthetic construct
  REGION 1..7: note = Description of Artificial Sequence: Syntheticpeptide
KHSTRRV

SEQ ID NO: 62 - protein, 7 aa, synthetic construct
  REGION 1..7: note = Description of Artificial Sequence: Syntheticpeptide
TMHQRVE

SEQ ID NO: 63 - protein, 7 aa, synthetic construct
  REGION 1..7: note = Description of Artificial Sequence: Syntheticpeptide
TSGHLSR

SEQ ID NO: 64 - protein, 7 aa, synthetic construct
  REGION 1..7: note = Description of Artificial Sequence: Syntheticpeptide
RSDHLTQ

SEQ ID NO: 65 - protein, 7 aa, synthetic construct
  REGION 1..7: note = Description of Artificial Sequence: Syntheticpeptide
DSANLSR

SEQ ID NO: 66 - protein, 7 aa, synthetic construct
  REGION 1..7: note = Description of Artificial Sequence: Syntheticpeptide
QSGSLTR

SEQ ID NO: 67 - protein, 7 aa, synthetic construct
  REGION 1..7: note = Description of Artificial Sequence: Syntheticpeptide
AKWNLDA

SEQ ID NO: 68 - protein, 7 aa, synthetic construct
  REGION 1..7: note = Description of Artificial Sequence: Syntheticpeptide
RNDSLKT

SEQ ID NO: 69 - protein, 7 aa, synthetic construct
  REGION 1..7: note = Description of Artificial Sequence: Syntheticpeptide
DSSNLSR

SEQ ID NO: 70 - protein, 7 aa, synthetic construct
  REGION 1..7: note = Description of Artificial Sequence: Syntheticpeptide
QKATRTT

SEQ ID NO: 71 - protein, 7 aa, synthetic construct
  REGION 1..7: note = Description of Artificial Sequence: Syntheticpeptide
TSSNRTK

SEQ ID NO: 72 - protein, 7 aa, synthetic construct
  REGION 1..7: note = Description of Artificial Sequence: Syntheticpeptide
RNASRTR

SEQ ID NO: 73 - protein, 7 aa, synthetic construct
  REGION 1..7: note = Description of Artificial Sequence: Syntheticpeptide
QSSDLRR

SEQ ID NO: 74 - protein, 7 aa, synthetic construct
  REGION 1..7: note = Description of Artificial Sequence: Syntheticpeptide
RSDNLSE

SEQ ID NO: 75 - protein, 7 aa, synthetic construct
  REGION 1..7: note = Description of Artificial Sequence: Syntheticpeptide
ERANRNS

SEQ ID NO: 76 - protein, 7 aa, synthetic construct
  REGION 1..7: note = Description of Artificial Sequence: Syntheticpeptide
RSDNLAR

SEQ ID NO: 77 - protein, 7 aa, synthetic construct
  REGION 1..7: note = Description of Artificial Sequence: Syntheticpeptide
QKVNLMS

SEQ ID NO: 78 - protein, 7 aa, synthetic construct
  REGION 1..7: note = Description of Artificial Sequence: Syntheticpeptide
QKVNLRE

SEQ ID NO: 79 - protein, 7 aa, synthetic construct
  REGION 1..7: note = Description of Artificial Sequence: Syntheticpeptide
RSDTLSQ

SEQ ID NO: 80 - protein, 7 aa, synthetic construct
  REGION 1..7: note = Description of Artificial Sequence: Syntheticpeptide
RKQTRTT

SEQ ID NO: 81 - protein, 7 aa, synthetic construct
  REGION 1..7: note = Description of Artificial Sequence: Syntheticpeptide
HRSSLRR

SEQ ID NO: 82 - protein, 7 aa, synthetic construct
  REGION 1..7: note = Description of Artificial Sequence: Syntheticpeptide
TSANLSR

SEQ ID NO: 83 - protein, 7 aa, synthetic construct
  REGION 1..7: note = Description of Artificial Sequence: Syntheticpeptide
WHSSLRV

SEQ ID NO: 84 - protein, 7 aa, synthetic construct
  REGION 1..7: note = Description of Artificial Sequence: Syntheticpeptide
RSAHLSR

SEQ ID NO: 85 - protein, 7 aa, synthetic construct
  REGION 1..7: note = Description of Artificial Sequence: Syntheticpeptide
RSDVLSV

SEQ ID NO: 86 - protein, 7 aa, synthetic construct
  REGION 1..7: note = Description of Artificial Sequence: Syntheticpeptide
QNNHRIT

SEQ ID NO: 87 - protein, 7 aa, synthetic construct
  REGION 1..7: note = Description of Artificial Sequence: Syntheticpeptide
SPSSRRT

SEQ ID NO: 88 - protein, 7 aa, synthetic construct
  REGION 1..7: note = Description of Artificial Sequence: Syntheticpeptide
RSDSLSV

SEQ ID NO: 89 - protein, 7 aa, synthetic construct
  REGION 1..7: note = Description of Artificial Sequence: Syntheticpeptide
QNANRKT

SEQ ID NO: 90 - protein, 7 aa, synthetic construct
  REGION 1..7: note = Description of Artificial Sequence: Syntheticpeptide
RSDSLST

SEQ ID NO: 91 - protein, 7 aa, synthetic construct
  REGION 1..7: note = Description of Artificial Sequence: Syntheticpeptide
DRANRIK

SEQ ID NO: 92 - DNA, 23 bp, Homo sapiens
  source 1..23: mol_type = other DNA
gctggtacacggcagggtcaggg

SEQ ID NO: 93 - DNA, 23 bp, Homo sapiens
  source 1..23: mol_type = other DNA
agagtctctcagctggtacacgg

SEQ ID NO: 94 - DNA, 23 bp, Homo sapiens
  source 1..23: mol_type = other DNA
gagaatcaaaatcggtgaatagg

SEQ ID NO: 95 - DNA, 23 bp, Homo sapiens
  source 1..23: mol_type = other DNA
acaaaactgtgctagacatgagg

SEQ ID NO: 96 - DNA, 23 bp, Homo sapiens
  source 1..23: mol_type = other DNA
agagcaacagtgctgtggcctgg

SEQ ID NO: 97 - DNA, 23 bp, Homo sapiens
  source 1..23: mol_type = other DNA
gacaccttcttccccagcccagg

SEQ ID NO: 98 - DNA, 23 bp, Homo sapiens
  source 1..23: mol_type = other DNA
ctcgaccagcttgacatcacagg

SEQ ID NO: 99 - DNA, 23 bp, Homo sapiens
  source 1..23: mol_type = other DNA
aagttcctgtgatgtcaagctgg

SEQ ID NO: 100 - DNA, 23 bp, Homo sapiens
  source 1..23: mol_type = other DNA
gtcgagaaaagctttgaaacagg

SEQ ID NO: 101 - DNA, 23 bp, Homo sapiens
  source 1..23: mol_type = other DNA
ttcggaacccaatcactgacagg

SEQ ID NO: 102 - DNA, 23 bp, Homo sapiens
  source 1..23: mol_type = other DNA
ccactttcaggaggaggattcgg

SEQ ID NO: 103 - DNA, 23 bp, Homo sapiens
  source 1..23: mol_type = other DNA
acccggccactttcaggaggagg

SEQ ID NO: 104 - DNA, 20 bp, synthetic construct
  misc_feature 1..20: note = Description of Artificial Sequence: Syntheticoligonucleotide
  source 1..20: mol_type = other DNA
gctggtacacggcagggtca

SEQ ID NO: 105 - DNA, 21 bp, synthetic construct
  misc_feature 1..21: note = Description of Artificial Sequence: Syntheticoligonucleotide
  source 1..21: mol_type = other DNA
gagagtctctcagctggtaca

SEQ ID NO: 106 - DNA, 20 bp, synthetic construct
  misc_feature 1..20: note = Description of Artificial Sequence: Syntheticoligonucleotide
  source 1..20: mol_type = other DNA
gagaatcaaaatcggtgaat

SEQ ID NO: 107 - DNA, 21 bp, synthetic construct
  misc_feature 1..21: note = Description of Artificial Sequence: Syntheticoligonucleotide
  source 1..21: mol_type = other DNA
gacaaaactgtgctagacatg

SEQ ID NO: 108 - DNA, 21 bp, synthetic construct
  misc_feature 1..21: note = Description of Artificial Sequence: Syntheticoligonucleotide
  source 1..21: mol_type = other DNA
gagagcaacagtgctgtggcc

SEQ ID NO: 109 - DNA, 20 bp, synthetic construct
  misc_feature 1..20: note = Description of Artificial Sequence: Syntheticoligonucleotide
  source 1..20: mol_type = other DNA
gacaccttcttccccagccc

SEQ ID NO: 110 - DNA, 21 bp, synthetic construct
  misc_feature 1..21: note = Description of Artificial Sequence: Syntheticoligonucleotide
  source 1..21: mol_type = other DNA
gctcgaccagcttgacatcac

SEQ ID NO: 111 - DNA, 21 bp, synthetic construct
  misc_feature 1..21: note = Description of Artificial Sequence: Syntheticoligonucleotide
  source 1..21: mol_type = other DNA
gaagttcctgtgatgtcaagc

SEQ ID NO: 112 - DNA, 20 bp, synthetic construct
  misc_feature 1..20: note = Description of Artificial Sequence: Syntheticoligonucleotide
  source 1..20: mol_type = other DNA
gtcgagaaaagctttgaaac

SEQ ID NO: 113 - DNA, 21 bp, synthetic construct
  misc_feature 1..21: note = Description of Artificial Sequence: Syntheticoligonucleotide
  source 1..21: mol_type = other DNA
gttcggaacccaatcactgac

SEQ ID NO: 114 - DNA, 21 bp, synthetic construct
  misc_feature 1..21: note = Description of Artificial Sequence: Syntheticoligonucleotide
  source 1..21: mol_type = other DNA
gccactttcaggaggaggatt

SEQ ID NO: 115 - DNA, 21 bp, synthetic construct
  misc_feature 1..21: note = Description of Artificial Sequence: Syntheticoligonucleotide
  source 1..21: mol_type = other DNA
gacccggccactttcaggagg

SEQ ID NO: 116 - DNA, 300 bp, Homo sapiens
  source 1..300: mol_type = other DNA
atatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcag

SEQ ID NO: 117 - DNA, 28 bp, synthetic construct
  misc_feature 1..28: note = Description of Artificial Sequence: Syntheticoligonucleotide
  source 1..28: mol_type = other DNA
tgtcggatggatgaaacccagacacata

SEQ ID NO: 118 - DNA, 160 bp, Homo sapiens
  source 1..160: mol_type = other DNA
aagcccataaccgctgtggcctcttggttttacagatacgaacctaaactttcaaaacctgtcagtgattgggttccgaatcctcctcctgaaagtggccgggtttaatctgctcatgacgctgcggctgtggtccagctgaggtgaggggccttgaagc

SEQ ID NO: 119 - protein, 7 aa, synthetic construct
  REGION 1..7: note = Description of Artificial Sequence: Syntheticpeptide
QQHVLQN

SEQ ID NO: 120 - protein, 7 aa, synthetic construct
  REGION 1..7: note = Description of Artificial Sequence: Syntheticpeptide
ASKTRTN

SEQ ID NO: 121 - protein, 7 aa, synthetic construct
  REGION 1..7: note = Description of Artificial Sequence: Syntheticpeptide
RTEDRLA

SEQ ID NO: 122 - protein, 9 aa, unidentified
  REGION 1..9: note = Description of Unknown:"LAGLIDADG" family peptide motif sequence
LAGLIDADG

SEQ ID NO: 123 - DNA, 28 bp, synthetic construct
  misc_feature 1..28: note = Description of Artificial Sequence: Syntheticoligonucleotide
  source 1..28: mol_type = other DNA
tagcaattcaggaaatttgactttccat

SEQ ID NO: 124 - skipped

SEQ ID NO: 125 - protein, 7 aa, synthetic construct
  REGION 1..7: note = Description of Artificial Sequence: Syntheticpeptide
ARWYLDK

SEQ ID NO: 126 - DNA, 28 bp, synthetic construct
  misc_feature 1..28: note = Description of Artificial Sequence: Syntheticoligonucleotide
  source 1..28: mol_type = other DNA
gagtagcgcgagcacagctaaggccacg

SEQ ID NO: 127 - DNA, 28 bp, synthetic construct
  misc_feature 1..28: note = Description of Artificial Sequence: Syntheticoligonucleotide
  source 1..28: mol_type = other DNA
gccacggagcgagacatctcggcccgaa

SEQ ID NO: 128 - protein, 7 aa, synthetic construct
  REGION 1..7: note = Description of Artificial Sequence: Syntheticpeptide
AQCCLFH

SEQ ID NO: 129 - protein, 7 aa, synthetic construct
  REGION 1..7: note = Description of Artificial Sequence: Syntheticpeptide
RSANLTR

SEQ ID NO: 130 - protein, 7 aa, synthetic construct
  REGION 1..7: note = Description of Artificial Sequence: Syntheticpeptide
RSDDLTR

SEQ ID NO: 131 - protein, 7 aa, synthetic construct
  REGION 1..7: note = Description of Artificial Sequence: Syntheticpeptide
RSDDLSK

SEQ ID NO: 132 - protein, 7 aa, synthetic construct
  REGION 1..7: note = Description of Artificial Sequence: Syntheticpeptide
DSSARKK

SEQ ID NO: 133 - protein, 7 aa, synthetic construct
  REGION 1..7: note = Description of Artificial Sequence: Syntheticpeptide
QRTHLRD

SEQ ID NO: 134 - protein, 7 aa, synthetic construct
  REGION 1..7: note = Description of Artificial Sequence: Syntheticpeptide
DSSNREA

SEQ ID NO: 135 - protein, 9 aa, synthetic construct
  REGION 1..9: note = Description of Artificial Sequence: Syntheticpeptide
LRGSQLVKS

SEQ ID NO: 136 - protein, 12 aa, synthetic construct
  REGION 1..12: note = Description of Artificial Sequence: Syntheticpeptide
SGAIRCHDEFWF

SEQ ID NO: 137 - protein, 12 aa, synthetic construct
  REGION 1..12: note = Description of Artificial Sequence: Syntheticpeptide
SGTPHEVGVYTL

SEQ ID NO: 138 - protein, 579 aa, unidentified
  REGION 1..579: note = Description of Unknown:FokI protein sequence
MVSKIRTFGWVQNPGKFENLKRVVQVFDRNSKVHNEVKNIKIPTLVKESKIQKELVAIMNQHDLIYTYKELVGTGTSIRSEAPCDAIIQATIADQGNKKGYIDNWSSDGFLRWAHALGFIEYINKSDSFVITDVGLAYSKSADGSAIEKEILIEAISSYPPAIRILTLLEDGQHLTKFDLGKNLGFSGESGFTSLPEGILLDTLANAMPKDKGEIRNNWEGSSDKYARMIGGWLDKLGLVKQGKKEFIIPTLGKPDNKEFISHAFKITGEGLKVLRRAKGSTKFTRVPKRVYWEMLATNLTDKEYVRTRRALILEILIKAGSLKIEQIQDNLKKLGFDEVIETIENDIKGLINTGIFIEIKGRFYQLKDHILQFVIPNRGVTKQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 139 - protein, 196 aa, unidentified
  REGION 1..196: note = Description of Unknown:FokI cleavage domain
QLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 140 - protein, 196 aa, synthetic construct
  REGION 1..196: note = Description of Artificial Sequence: Syntheticpolypeptide
QLVKSELEEKKSELRHKLKYVPHEYIELIEIARNPTQDRILEMKVMEFFMKVYGYRGEHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 141 - protein, 196 aa, synthetic construct
  REGION 1..196: note = Description of Artificial Sequence: Syntheticpolypeptide
QLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMERYVEENQTRDKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 142 - protein, 196 aa, synthetic construct
  REGION 1..196: note = Description of Artificial Sequence: Syntheticpolypeptide
QLVKSELEEKKSELRHKLKYVPHEYIELIEIARNPTQDRILEMKVMEFFMKVYGYRGEHLGGSRKPDGAIYTVGSPIDYGVIVDTKAYSGGYNLPIGQADEMERYVEENQTRDKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 143 - protein, 196 aa, synthetic construct
  REGION 1..196: note = Description of Artificial Sequence: Syntheticpolypeptide
QLVKSELEEKKSELRHKLKYVPHEYIELIEIAENSTQDRILEMKVMEFFMKVYGYRGKHLGGSRKPDGAIYTVGSPIDYGVIVDTKAYSGGYNLPIGQADEMERYVEENQTRDKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 144 - protein, 196 aa, synthetic construct
  REGION 1..196: note = Description of Artificial Sequence: Syntheticpolypeptide
QLVKSELEEKKSELRHKLKYVPHEYIELIEIAENPTQDRILEMKVMEFFMKVYGYRGEHLGGSRKPDGAIYTVGSPIDYGVIVDTKAYSGGYNLPIGQADEMERYVEENQTRDKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 145 - protein, 196 aa, synthetic construct
  REGION 1..196: note = Description of Artificial Sequence: Syntheticpolypeptide
QLVKSELEEKKSELRHKLKYVPHEYIELIEIAYNSTQDRILEMKVMEFFMKVYGYRGKHLGGSRKPDGAIYTVGSPIDYGVIVDTKAYSGGYNLPIGQADEMERYVEENQTRDKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 146 - protein, 196 aa, synthetic construct
  REGION 1..196: note = Description of Artificial Sequence: Syntheticpolypeptide
QLVKSELEEKKSELRHKLKYVPHEYIELIEIAYNPTQDRILEMKVMEFFMKVYGYRGEHLGGSRKPDGAIYTVGSPIDYGVIVDTKAYSGGYNLPIGQADEMERYVEENQTRDKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 147 - protein, 196 aa, synthetic construct
  REGION 1..196: note = Description of Artificial Sequence: Syntheticpolypeptide
QLVKSELEEKKSELRHKLKYVPHEYIELIEIARNETQDRILEMKVMEFFMKVYGYRGKHLGGSRKPDGAIYTVGSPIDYGVIVDTKAYSGGYNLPIGQADEMERYVEENQTRDKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 148 - protein, 196 aa, synthetic construct
  REGION 1..196: note = Description of Artificial Sequence: Syntheticpolypeptide
QLVKSELEEKKSELRHKLKYVPHEYIELIEIARNETQDRILEMKVMEFFMKVYGYRGEHLGGSRKPDGAIYTVGSPIDYGVIVDTKAYSGGYNLPIGQADEMERYVEENQTRDKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 149 - protein, 196 aa, synthetic construct
  REGION 1..196: note = Description of Artificial Sequence: Syntheticpolypeptide
QLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMERYVEENQTRDKHLNPNEWWKVYPSSVTEFKFLFVSGHFSGNYKAQLTRLNHITNCNGAVLSVEELLIGGEMIKAGTLTLEEVRRKFNNGEINF

SEQ ID NO: 150 - protein, 196 aa, synthetic construct
  REGION 1..196: note = Description of Artificial Sequence: Syntheticpolypeptide
QLVKSELEEKKSELRHKLKYVPHEYIELIEIARNPTQDRILEMKVMEFFMKVYGYRGEHLGGSRKPDGAIYTVGSPIDYGVIVDTKAYSGGYNLPIGQADEMERYVEENQTRDKHLNPNEWWKVYPSSVTEFKFLFVSGHFSGNYKAQLTRLNHITNCNGAVLSVEELLIGGEMIKAGTLTLEEVRRKFNNGEINF

SEQ ID NO: 151 - protein, 196 aa, synthetic construct
  REGION 1..196: note = Description of Artificial Sequence: Syntheticpolypeptide
QLVKSELEEKKSELRHKLKYVPHEYIELIEIARNSTQDRILEMKVMEFFMKVYGYRGKHLGGSRKPDGAIYTVGSPIDYGVIVDTKAYSGGYNLPTGQADEMERYVEENQTRDKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 152 - protein, 196 aa, synthetic construct
  REGION 1..196: note = Description of Artificial Sequence: Syntheticpolypeptide
QLVKSELEEKKSELRHKLKYVPHEYIELIEIARNPTQDRILEMKVMEFFMKVYGYRGEHLGGSRKPDGAIYTVGSPIDYGVIVDTKAYSGGYNLPTGQADEMERYVEENQTRDKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 153 - protein, 196 aa, synthetic construct
  REGION 1..196: note = Description of Artificial Sequence: Syntheticpolypeptide
QLVKSELEEKKSELRHKLKYVPHEYIELIEIARNSTQDRILEMKVMEFFMKVYGYRGKHLGGSRKPDGAIYTVGSPIDYGVIVDTKAYSGGYNLDIGQADEMERYVEENQTRDKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 154 - protein, 196 aa, synthetic construct
  REGION 1..196: note = Description of Artificial Sequence: Syntheticpolypeptide
QLVKSELEEKKSELRHKLKYVPHEYIELIEIARNPTQDRILEMKVMEFFMKVYGYRGEHLGGSRKPDGAIYTVGSPIDYGVIVDTKAYSGGYNLDIGQADEMERYVEENQTRDKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 155 - protein, 196 aa, synthetic construct
  REGION 1..196: note = Description of Artificial Sequence: Syntheticpolypeptide
QLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGDADEMERYVEENQTRDKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 156 - protein, 196 aa, synthetic construct
  REGION 1..196: note = Description of Artificial Sequence: Syntheticpolypeptide
QLVKSELEEKKSELRHKLKYVPHEYIELIEIARNPTQDRILEMKVMEFFMKVYGYRGEHLGGSRKPDGAIYTVGSPIDYGVIVDTKAYSGGYNLPIGDADEMERYVEENQTRDKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 157 - protein, 196 aa, synthetic construct
  REGION 1..196: note = Description of Artificial Sequence: Syntheticpolypeptide
QLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVKENQTRNKHINPNEWWKVYPSSVTEFKFLFVSGHFKGNYKAQLTRLNRKTNCNGAVLSVEELLIGGEMIKAGTLTLEEVRRKFNNGEINF

SEQ ID NO: 158 - protein, 196 aa, synthetic construct
  REGION 1..196: note = Description of Artificial Sequence: Syntheticpolypeptide
QLVKSELEEKKSELRHKLKYVPHEYIELIEIARNPTQDRILEMKVMEFFMKVYGYRGEHLGGSRKPDGAIYTVGSPIDYGVIVDTKAYSGGYNLPIGQADEMQRYVKENQTRNKHINPNEWWKVYPSSVTEFKFLFVSGHFKGNYKAQLTRLNRKTNCNGAVLSVEELLIGGEMIKAGTLTLEEVRRKFNNGEINF

SEQ ID NO: 159 - protein, 196 aa, synthetic construct
  REGION 1..196: note = Description of Artificial Sequence: Syntheticpolypeptide
QLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGEADEMQRYVKENQTRNKHINPNEWWKVYPSSVTEFKFLFVSGHFKGNYKAQLTRLNRKTNCNGAVLSVEELLIGGEMIKAGTLTLEEVRRKFNNGEINF

SEQ ID NO: 160 - protein, 196 aa, synthetic construct
  REGION 1..196: note = Description of Artificial Sequence: Syntheticpolypeptide
QLVKSELEEKKSELRHKLKYVPHEYIELIEIARNPTQDRILEMKVMEFFMKVYGYRGEHLGGSRKPDGAIYTVGSPIDYGVIVDTKAYSGGYNLPIGEADEMQRYVKENQTRNKHINPNEWWKVYPSSVTEFKFLFVSGHFKGNYKAQLTRLNRKTNCNGAVLSVEELLIGGEMIKAGTLTLEEVRRKFNNGEINF

SEQ ID NO: 161 - protein, 196 aa, synthetic construct
  REGION 1..196: note = Description of Artificial Sequence: Syntheticpolypeptide
QLVKSELEEKKSELRHKLKYVPHEYIELIEIAENSTQDRILEMKVMEFFMKVYGYRGKHLGGSRKPDGAIYTVGSPIDYGVIVDTKAYSGGYNLPIGQADEMQRYVKENQTRNKHINPNEWWKVYPSSVTEFKFLFVSGHFKGNYKAQLTRLNRKTNCNGAVLSVEELLIGGEMIKAGTLTLEEVRRKFNNGEINF

SEQ ID NO: 162 - protein, 196 aa, synthetic construct
  REGION 1..196: note = Description of Artificial Sequence: Syntheticpolypeptide
QLVKSELEEKKSELRHKLKYVPHEYIELIEIAENPTQDRILEMKVMEFFMKVYGYRGEHLGGSRKPDGAIYTVGSPIDYGVIVDTKAYSGGYNLPIGQADEMQRYVKENQTRNKHINPNEWWKVYPSSVTEFKFLFVSGHFKGNYKAQLTRLNRKTNCNGAVLSVEELLIGGEMIKAGTLTLEEVRRKFNNGEINF

SEQ ID NO: 163 - protein, 196 aa, synthetic construct
  REGION 1..196: note = Description of Artificial Sequence: Syntheticpolypeptide
QLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVKENQTRNKHINPNEWWKVYPSSVTEFKFLFVSGHFSGNYKAQLTRLNRKTNCNGAVLSVEELLIGGEMIKAGTLTLEEVRRKFNNGEINF

SEQ ID NO: 164 - protein, 196 aa, synthetic construct
  REGION 1..196: note = Description of Artificial Sequence: Syntheticpolypeptide
QLVKSELEEKKSELRHKLKYVPHEYIELIEIARNPTQDRILEMKVMEFFMKVYGYRGEHLGGSRKPDGAIYTVGSPIDYGVIVDTKAYSGGYNLPIGQADEMQRYVKENQTRNKHINPNEWWKVYPSSVTEFKFLFVSGHFSGNYKAQLTRLNRKTNCNGAVLSVEELLIGGEMIKAGTLTLEEVRRKFNNGEINF

SEQ ID NO: 165 - protein, 196 aa, synthetic construct
  REGION 1..196: note = Description of Artificial Sequence: Syntheticpolypeptide
QLVKSELEEKKSELRHKLKYVPHEYIELIEIAYNSTQDRILEMKVMEFFMKVYGYRGKHLGGSRKPDGAIYTVGSPIDYGVIVDTKAYSGGYNLPIGQADEMQRYVKENQTRNKHINPNEWWKVYPSSVTEFKFLFVSGHFKGNYKAQLTRLNRKTNCNGAVLSVEELLIGGEMIKAGTLTLEEVRRKFNNGEINF

SEQ ID NO: 166 - protein, 196 aa, synthetic construct
  REGION 1..196: note = Description of Artificial Sequence: Syntheticpolypeptide
QLVKSELEEKKSELRHKLKYVPHEYIELIEIAYNPTQDRILEMKVMEFFMKVYGYRGEHLGGSRKPDGAIYTVGSPIDYGVIVDTKAYSGGYNLPIGQADEMQRYVKENQTRNKHINPNEWWKVYPSSVTEFKFLFVSGHFKGNYKAQLTRLNRKTNCNGAVLSVEELLIGGEMIKAGTLTLEEVRRKFNNGEINF

SEQ ID NO: 167 - protein, 196 aa, synthetic construct
  REGION 1..196: note = Description of Artificial Sequence: Syntheticpolypeptide
QLVKSELEEKKSELRHKLKYVPHEYIELIEIARNSTQDRILEMKVMEFFMKVYGYRGKHLGGSRKPDGAIYTVGSPIDYGVIVDTKAYSGGYNLPTGQADEMQRYVKENQTRNKHINPNEWWKVYPSSVTEFKFLFVSGHFKGNYKAQLTRLNRKTNCNGAVLSVEELLIGGEMIKAGTLTLEEVRRKFNNGEINF

SEQ ID NO: 168 - protein, 196 aa, synthetic construct
  REGION 1..196: note = Description of Artificial Sequence: Syntheticpolypeptide
QLVKSELEEKKSELRHKLKYVPHEYIELIEIARNPTQDRILEMKVMEFFMKVYGYRGEHLGGSRKPDGAIYTVGSPIDYGVIVDTKAYSGGYNLPTGQADEMQRYVKENQTRNKHINPNEWWKVYPSSVTEFKFLFVSGHFKGNYKAQLTRLNRKTNCNGAVLSVEELLIGGEMIKAGTLTLEEVRRKFNNGEINF

SEQ ID NO: 169 - protein, 196 aa, synthetic construct
  REGION 1..196: note = Description of Artificial Sequence: Syntheticpolypeptide
QLVKSELEEKKSELRHKLKYVPHEYIELIEIARNSTQDRILEMKVMEFFMKVYGYRGKHLGGSRKPDGAIYTVGSPIDYGVIVDTKAYSGGYNLDIGQADEMQRYVKENQTRNKHINPNEWWKVYPSSVTEFKFLFVSGHFKGNYKAQLTRLNRKTNCNGAVLSVEELLIGGEMIKAGTLTLEEVRRKFNNGEINF

SEQ ID NO: 170 - protein, 196 aa, synthetic construct
  REGION 1..196: note = Description of Artificial Sequence: Syntheticpolypeptide
QLVKSELEEKKSELRHKLKYVPHEYIELIEIARNPTQDRILEMKVMEFFMKVYGYRGEHLGGSRKPDGAIYTVGSPIDYGVIVDTKAYSGGYNLDIGQADEMQRYVKENQTRNKHINPNEWWKVYPSSVTEFKFLFVSGHFKGNYKAQLTRLNRKTNCNGAVLSVEELLIGGEMIKAGTLTLEEVRRKFNNGEINF

SEQ ID NO: 171 - protein, 196 aa, synthetic construct
  REGION 1..196: note = Description of Artificial Sequence: Syntheticpolypeptide
QLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQDYVEENQTRDKHANPNEWWKVYPSSVTEFKFLFVSGHFKGNYKAQLTRLNHITNCNGAVLSVEELLIGGEMIKAGTLTLEEVRRKFNNGEINF

SEQ ID NO: 172 - protein, 196 aa, synthetic construct
  REGION 1..196: note = Description of Artificial Sequence: Syntheticpolypeptide
QLVKSELEEKKSELRHKLKYVPHEYIELIEIARNPTQDRILEMKVMEFFMKVYGYRGEHLGGSRKPDGAIYTVGSPIDYGVIVDTKAYSGGYNLPIGQADEMQDYVEENQTRDKHANPNEWWKVYPSSVTEFKFLFVSGHFKGNYKAQLTRLNHITNCNGAVLSVEELLIGGEMIKAGTLTLEEVRRKFNNGEINF

SEQ ID NO: 173 - protein, 196 aa, synthetic construct
  REGION 1..196: note = Description of Artificial Sequence: Syntheticpolypeptide
QLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQAREMQRYVEENQTRNKHINPNEWWKVYPSSVTEFKFLFVSGHFKGNYKAQLTRLNRVTNCNGAVLSVEELLIGGEMIKAGTLTLEEVRRKFNNGEINF

SEQ ID NO: 174 - protein, 196 aa, synthetic construct
  REGION 1..196: note = Description of Artificial Sequence: Syntheticpolypeptide
QLVKSELEEKKSELRHKLKYVPHEYIELIEIARNPTQDRILEMKVMEFFMKVYGYRGEHLGGSRKPDGAIYTVGSPIDYGVIVDTKAYSGGYNLPIGQAREMQRYVEENQTRNKHINPNEWWKVYPSSVTEFKFLFVSGHFKGNYKAQLTRLNRVTNCNGAVLSVEELLIGGEMIKAGTLTLEEVRRKFNNGEINF

SEQ ID NO: 175 - protein, 170 aa, synthetic construct
  REGION 1..170: note = Description of Artificial Sequence: Syntheticpolypeptide
RPFQCRICMRNFSRSDDLSKHIRTHTGEKPFACDICGRKFADSSARKKHTKIHTGEKPFQCRICMRNFSDRSNLSRHIRTHTGEKPFACDICGRKFAQRTHLRDHTKIHTHPRAPIPKPFQCRICMRNFSQSGHLARHIRTHTGEKPFACDICGRKFADSSNREAHTKIH

SEQ ID NO: 176 - protein, 142 aa, synthetic construct
  REGION 1..142: note = Description of Artificial Sequence: Syntheticpolypeptide
RPFQCRICMRKFAAQCCLFHHTKIHTGEKPFQCRICMRNFSDQSNLRAHIRTHTGEKPFACDICGRKFARSANLTRHTKIHTHPRAPIPKPFQCRICMRNFSRSDDLTRHIRTHTGEKPFACDICGRKFAQSGSLTRHTKIH

SEQ ID NO: 177 - protein, 171 aa, synthetic construct
  REGION 1..171: note = Description of Artificial Sequence: Syntheticpolypeptide
RPFQCRICMQNFSQSSDLSRHIRTHTGEKPFACDICGRKFAQSGNRTTHTKIHTHPRAPIPKPFQCRICMQNFSRSANLARHIRTHTGEKPFACDICGRKFADRSALARHTKIHTGSQKPFQCRICMQNFSRSDVLSEHIRTHTGEKPFACDICGRKFAKHSTRRVHTKIH

SEQ ID NO: 178 - protein, 171 aa, synthetic construct
  REGION 1..171: note = Description of Artificial Sequence: Syntheticpolypeptide
RPFQCRICMRNFSTMHQRVEHIRTHTGEKPFACDICGRKFATSGHLSRHTKIHTGSQKPFQCRICMRNFSRSDHLTQHIRTHTGEKPFACDICGRKFADSANLSRHTKIHTHPRAPIPKPFQCRICMRNFSQSGSLTRHIRTHTGEKPFACDICGRKFAAKWNLDAHTKIH

SEQ ID NO: 179 - protein, 12 aa, synthetic construct
  REGION 1..12: note = Description of Artificial Sequence: Syntheticpeptide
SGAIRCHDEFWF

SEQ ID NO: 180 - protein, 22 aa, synthetic construct
  REGION 1..22: note = Description of Artificial Sequence: Syntheticpeptide
DYKDHDGDYKDHDIDYKDDDDK

SEQ ID NO: 181 - protein, 7 aa, unidentified
  REGION 1..7: note = Description of Unknown:Nuclear localization sequence
PKKKRKV

SEQ ID NO: 182 - DNA, 2601 bp, synthetic construct
  misc_feature 1..2601: note = Description of Artificial Sequence: Syntheticpolynucleotide
  source 1..2601: mol_type = other DNA
atggactacaaagaccatgacggtgattataaagatcatgacatcgattacaaggatgacgatgacaagatggcccccaagaagaagaggaaggtcggcatccacggggtacccgccgctatgggacagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctaccggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcaatggcgccgtgctgagcgtggaggagctgctgatcggcggcgagatgatcaaagccggcaccctgacactggaggaggtgcggcgcaagttcaacaacggcgagatcaacttcagcggcgccatcagatgccacgacgagttctggttcaggcccttccagtgtcgaatctgcatgcagaacttcagtcagtcctccgacctgtcccgccacatccgcacccacaccggcgagaagccttttgcctgtgacatttgtgggaggaaatttgcccagtccggcaaccgcaccacccataccaagatacacacgcatcccagggcacctattcccaagcccttccagtgtcgaatctgcatgcagaacttcagtcgctccgccaacctggcccgccacatccgcacccacaccggcgagaagccttttgcctgtgacatttgtgggaggaaatttgccgaccgctccgccctggcccgccataccaagatacacacgggatctcagaagcccttccagtgtcgaatctgcatgcagaacttcagtcgctccgacgtgctgtccgagcacatccgcacccacaccggcgagaagccttttgcctgtgacatttgtgggaggaaatttgccaagcactccacccgccgcgtgcataccaagatacacctgcggcagaaggacagatctggcggcggagagggcagaggaagtcttctaacctgcggtgacgtggaggagaatcccggccctaggaccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcgtaacttcagtaccatgcaccagcgcgtggagcacatccgcacccacaccggcgagaagcctttcgcctgtgacatttgtgggaggaaatttgccacctccggccacctgtcccgccataccaagatacacacgggcagccaaaagcccttccagtgtcgaatctgcatgcgtaacttcagtcgctccgaccacctgacccagcacatccgcacccacaccggcgagaagccttttgcctgtgacatttgtgggaggaaatttgccgactccgccaacctgtcccgccataccaagatacacacgcacccgcgcgccccgatcccgaagcccttccagtgtcgaatctgcatgcgtaacttcagtcagtccggctccctgacccgccacatccgcacccacaccggcgagaagccttttgcctgtgacatttgtgggaggaaatttgccgccaagtggaacctggacgcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttctgataa

SEQ ID NO: 183 - protein, 865 aa, synthetic construct
  REGION 1..865: note = Description of Artificial Sequence: Syntheticpolypeptide
MDYKDHDGDYKDHDIDYKDDDDKMAPKKKRKVGIHGVPAAMGQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPTGQADEMERYVEENQTRDKHLNPNEWWKVYPSSVTEFKFLFVSGHFKGNYKAQLTRLNHITNCNGAVLSVEELLIGGEMIKAGTLTLEEVRRKFNNGEINFSGAIRCHDEFWFRPFQCRICMQNFSQSSDLSRHIRTHTGEKPFACDICGRKFAQSGNRTTHTKIHTHPRAPIPKPFQCRICMQNFSRSANLARHIRTHTGEKPFACDICGRKFADRSALARHTKIHTGSQKPFQCRICMQNFSRSDVLSEHIRTHTGEKPFACDICGRKFAKHSTRRVHTKIHLRQKDRSGGGEGRGSLLTCGDVEENPGPRTMDYKDHDGDYKDHDIDYKDDDDKMAPKKKRKVGIHGVPAAMAERPFQCRICMRNFSTMHQRVEHIRTHTGEKPFACDICGRKFATSGHLSRHTKIHTGSQKPFQCRICMRNFSRSDHLTQHIRTHTGEKPFACDICGRKFADSANLSRHTKIHTHPRAPIPKPFQCRICMRNFSQSGSLTRHIRTHTGEKPFACDICGRKFAAKWNLDAHTKIHLRGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVKENQTRNKHINPNEWWKVYPSSVTEFKFLFVSGHFKGNYKAQLTRLNRKTNCNGAVLSVEELLIGGEMIKAGTLTLEEVRRKFNNGEINF

SEQ ID NO: 184 - DNA, 2505 bp, synthetic construct
  misc_feature 1..2505: note = Description of Artificial Sequence: Syntheticpolynucleotide
  source 1..2505: mol_type = other DNA
atggactacaaagaccatgacggtgattataaagatcatgacatcgattacaaggatgacgatgacaagatggcccccaagaagaagaggaaggtcggcatccacggggtacccgccgctatggctgagaggcccttccagtgtcgaatctgcatgcgtaacttcagtcgtagtgacgacctgagcaagcacatccgcacccacacaggcgagaagccttttgcctgtgacatttgtgggaggaaatttgccgacagcagcgcccgcaaaaagcataccaagatacacacgggcgagaagcccttccagtgtcgaatctgcatgcgtaacttcagtgaccgctccaacctgtcccgccacatccgcacccacaccggcgagaagccttttgcctgtgacatttgtgggaggaaatttgcccagcgcacccacctgcgcgaccataccaagatacacacgcacccgcgcgccccgatcccgaagcccttccagtgtcgaatctgcatgcgtaacttcagtcagtccggccacctggcccgccacatccgcacccacaccggcgagaagccttttgcctgtgacatttgtgggaggaaatttgccgactcctccaaccgcgaggcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctggacatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcaatggcgccgtgctgagcgtggaggagctgctgatcggcggcgagatgatcaaagccggcaccctgacactggaggaggtgcggcgcaagttcaacaacggcgagatcaacttcagatctggcggcggagagggcagaggaagtcttctaacctgcggtgacgtggaggagaatcccggccctaggaccatggactacaaagaccatgacggtgattataaagatcatgacatcgattacaaggatgacgatgacaagatggcccccaagaagaagaggaaggtcggcattcatggggtacccgccgctatgggacagctggtgaagagcgagctggaggagaagaagtccgagctgcggcacaagctgaagtacgtgccccacgagtacatcgagctgatcgagatcgcctacaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttcagcggcgctcagggatctaccctggactttaggcccttccagtgtcgaatctgcatgcgtaagtttgccgcccagtgttgtctgttccaccataccaagatacacacgggcgagaagcccttccagtgtcgaatctgcatgcgtaacttcagtgaccagtccaacctgcgcgcccacatccgcacccacaccggcgagaagccttttgcctgtgacatttgtgggaggaaatttgcccgctccgccaacctgacccgccataccaagatacacacgcacccgcgcgccccgatcccgaagcccttccagtgtcgaatctgcatgcgtaacttcagtcgctccgacgacctgacccgccacatccgcacccacaccggcgagaagccttttgcctgtgacatttgtgggaggaaatttgcccagtccggctccctgacccgccataccaagatacacctgcggcagaaggactgataa

SEQ ID NO: 185 - protein, 833 aa, synthetic construct
  REGION 1..833: note = Description of Artificial Sequence: Syntheticpolypeptide
MDYKDHDGDYKDHDIDYKDDDDKMAPKKKRKVGIHGVPAAMAERPFQCRICMRNFSRSDDLSKHIRTHTGEKPFACDICGRKFADSSARKKHTKIHTGEKPFQCRICMRNFSDRSNLSRHIRTHTGEKPFACDICGRKFAQRTHLRDHTKIHTHPRAPIPKPFQCRICMRNFSQSGHLARHIRTHTGEKPFACDICGRKFADSSNREAHTKIHLRGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLDIGQADEMERYVEENQTRDKHLNPNEWWKVYPSSVTEFKFLFVSGHFKGNYKAQLTRLNHITNCNGAVLSVEELLIGGEMIKAGTLTLEEVRRKFNNGEINFRSGGGEGRGSLLTCGDVEENPGPRTMDYKDHDGDYKDHDIDYKDDDDKMAPKKKRKVGIHGVPAAMGQLVKSELEEKKSELRHKLKYVPHEYIELIEIAYNSTQDRILEMKVMEFFMKVYGYRGKHLGGSRKPDGAIYTVGSPIDYGVIVDTKAYSGGYNLPIGQADEMQRYVKENQTRNKHINPNEWWKVYPSSVTEFKFLFVSGHFKGNYKAQLTRLNRKTNCNGAVLSVEELLIGGEMIKAGTLTLEEVRRKFNNGEINFSGAQGSTLDFRPFQCRICMRKFAAQCCLFHHTKIHTGEKPFQCRICMRNFSDQSNLRAHIRTHTGEKPFACDICGRKFARSANLTRHTKIHTHPRAPIPKPFQCRICMRNFSRSDDLTRHIRTHTGEKPFACDICGRKFAQSGSLTRHTKIHLRQKD

SEQ ID NO: 186 - protein, 7 aa, synthetic construct
  REGION 1..7: note = Description of Artificial Sequence: Syntheticpeptide
RSDTLSE

SEQ ID NO: 187 - DNA, 100 bp, Homo sapiens
  source 1..100: mol_type = other DNA
tctggatgctgaaagaatgtctgtttttccttttagaaagttcctgtgatgtcaagctggtcgagaaaagctttgaaacaggtaagacaggggtctagcc

SEQ ID NO: 188 - DNA, 64 bp, synthetic construct
  misc_feature 1..64: note = Description of Artificial Sequence: Syntheticoligonucleotide
  source 1..64: mol_type = other DNA
aaaaaaaaaaaaaaaaaaaaaaaaaaaaaaaaaaaaaaaaaaaaaaaaaaaaaaaaaaaaaaaa

SEQ ID NO: 189 - DNA, 60 bp, synthetic construct
  misc_feature 1..60: note = Description of Artificial Sequence: Syntheticoligonucleotide
  source 1..60: mol_type = other DNA
aaaaaaaaaaaaaaaaaaaaaaaaaaaaaaaaaaaaaaaaaaaaaaaaaaaaaaaaaaaa

SEQ ID NO: 190 - protein, 4 aa, synthetic construct
  REGION 1..4: note = Description of Artificial Sequence: Syntheticpeptide
LRGS

(19) United States
(12) Patent Application Publication
(10) Pub. No.: US 2023/0295563 A1
(43) Pub. Date: Sep. 21, 2023

(54) TARGETED DISRUPTION OF T CELL AND/OR HLA RECEPTORS

(71) Applicant: Sangamo Therapeutics, Inc., Brisbane, CA (US)

(72) Inventors: Anthony Conway, Brisbane, CA (US); Sumiti Jain, Brisbane, CA (US); Gary K. Lee, Brisbane, CA (US); David Paschon, Brisbane, CA (US); Edward J. Rebar, Brisbane, CA (US); Lei Zhang, Brisbane, CA (US)

(21) Appl. No.: 17/971,071

(22) Filed: Oct. 21, 2022

Related U.S. Application Data
(62) Division of application No. 16/009,975, filed on Jun. 15, 2018, now Pat. No. 11,512,287.
(60) Provisional application No. 62/521,132, filed on Jun. 16, 2017.
(60) Provisional application No. 62/542,052, filed on Aug. 7, 2017.
(60) Provisional application No. 62/573,956, filed on Oct. 18, 2017.

(51) Int. Cl. C12N 5/0783; C12N 9/22; A61P 37/06; A61P 35/00; C07K 14/725; A61K 48/00; C12N 15/85; C12N 15/90; C07K 14/74

(52) U.S. Cl. CPC C12N 5/0636; A61K 48/0066; A61P 35/00; A61P 37/06; C07K 14/7051; C12N 9/22; C12N 15/907; C12N 15/8509; A61K 48/005; C07K 14/70539; C12N 2015/8518; C12N 2501/515; C12N 2510/00; C12N 2750/14143

(57) ABSTRACT

Disclosed herein are methods and compositions for inactivating TCR and/or HLA genes, using engineered nucleases comprising at least one DNA binding domain and a cleavage domain or cleavage half-domain in conditions able to preserve cell viability. Polynucleotides encoding nucleases, vectors comprising polynucleotides encoding nucleases and cells comprising polynucleotides encoding nucleases and/or cells comprising nucleases are also provided.

### CROSS-REFERENCE TO RELATED APPLICATIONS

The present application is a divisional application of U.S. Pat. Application No. 16/009,975, filed Jun. 15, 2018, which claims the benefit of U.S. Provisional Application No. 62/521,132, filed Jun. 16, 2017; U.S. Provisional Application 62/542,052, filed Aug. 7, 2017 and U.S. Provisional Application No. 62/573,956, filed Oct. 18, 2017, the disclosures of which are hereby incorporated by reference in their entireties.

### SEQUENCE LISTING

[0001.1] The instant application contains a Sequence Listing which has been submitted electronically in XML format and is hereby incorporated by reference in its entirety. Said XML copy, created on Mar. 9, 2023, is named 128687-2880.xml and is 248,720 bytes in size.

### TECHNICAL FIELD

The present disclosure is in the field of genome modification of human cells, including lymphocytes and stem cells.

### BACKGROUND

Gene therapy holds enormous potential for a new era of human therapeutics. These methodologies will allow treatment for conditions that have not been addressable by standard medical practice. Gene therapy can include the many variations of genome editing techniques such as disruption (inactivation) or correction of a gene locus, and/or insertion of an expressible transgene that can be controlled either by a specific exogenous promoter operably linked to the transgene, or by the endogenous promoter found at the site of insertion into the genome.

Delivery and insertion of the transgene are examples of hurdles that must be solved for any real implementation of this technology. For example, although a variety of gene delivery methods are potentially available for therapeutic use, all involve substantial tradeoffs between safety, durability and level of expression. Methods that provide the transgene as an episome (e.g., adenovirus (Ad), adeno-associated virus (AAV) and plasmid-based systems) can yield high initial expression levels, however, these methods lack robust episomal replication, which may limit the duration of expression in mitotically active tissues. In contrast, delivery methods that result in the random integration of the desired transgene (e.g., integrating lentivirus (LV)) provide more durable expression but, due to the untargeted nature of the random insertion, may provoke unregulated growth in the recipient cells, potentially leading to malignancy via activation of oncogenes in the vicinity of the randomly integrated transgene cassette. Moreover, although transgene integration avoids replication-driven loss, it does not prevent eventual silencing of the exogenous promoter fused to the transgene. Over time, such silencing results in reduced transgene expression for the majority of non-specific insertion events. In addition, integration of a transgene rarely occurs in every target cell, which can make it difficult to achieve a high enough expression level of the transgene of interest to achieve the desired therapeutic effect.

In recent years, a new strategy for genetic modification (e.g., inactivation, correction and/or transgene integration) has been developed that uses cleavage with site-specific nucleases (e.g., zinc finger nucleases (ZFNs), transcription activator-like effector domain nucleases (TALENs), CRISPR/Cas system with an engineered crRNA/tracr RNA (‘single guide RNA’) to guide specific cleavage, etc.) to bias editing at a chosen genomic locus. See, e.g., U.S. Pat. Nos. 9,937,207; 9,255,250; 9,045,763; 9,005,973; 8,956,828; 8,945,868; 8,703,489; 8,586,526; 6,534,261; 6,599,692; 6,503,717; 6,689,558; 7,067,317; 7,262,054; 7,888,121; 7,972,854; 7,914,796; 7,951,925; 8,110,379; 8,409,861; U.S. Patent Publication Nos. 2017/0211075; 2003/0232410; 2005/0208489; 2005/0026157; 2005/0064474; 2006/0063231; 2008/0159996; 2010/0218264; 2012/0017290; 2011/0265198; 2013/0137104; 2013/0122591; 2013/0177983 and 2013/0177960 and 2015/0056705. Further, targeted nucleases are being developed based on the Argonaute system (e.g., from T. thermophilus, known as ‘TtAgo’, see Swarts, et al. (2014) Nature 507(7491): 258-261), which also may have the potential for uses in genome editing and gene therapy. This nuclease-mediated approach to genetic modification offers the prospect of improved transgene expression, increased safety and expressional durability, as compared to classic integration approaches, since it allows exact transgene positioning for a minimal risk of gene silencing or activation of nearby oncogenes.

The T cell receptor (TCR) is an essential part of the selective activation of T cells. Bearing some resemblance to an antibody, the antigen recognition part of the TCR is typically made from two chains, α and β, which co-assemble to form a heterodimer. The antibody resemblance lies in the manner in which a single gene encoding a TCR alpha and beta complex is put together. TCR alpha (TCR α) and beta (TCR β) chains are each composed of two regions, a C-terminal constant region and an N-terminal variable region. The genomic loci that encode the TCR alpha and beta chains resemble antibody encoding loci in that the TCR α gene comprises V and J segments, while the β chain locus comprises D segments in addition to V and J segments. For the TCR β locus, there are additionally two different constant regions that are selected from during the selection process. During T cell development, the various segments recombine such that each T cell comprises a unique TCR variable portion in the alpha and beta chains, called the complementarity determining region (CDR), and the body has a large repertoire of T cells which, due to their unique CDRs, are capable of interacting with unique antigens displayed by antigen presenting cells. Once a TCR α or β gene rearrangement has occurred, the expression of the second corresponding TCR α or TCR β is repressed such that each T cell only expresses one unique TCR structure in a process called ‘antigen receptor allelic exclusion’ (see, Brady, et al. (2010) J Immunol 185:3801-3808).

During T cell activation, the TCR interacts with antigens displayed as peptides on the major histocompatability complex (MHC) of an antigen presenting cell. Recognition of the antigen-MHC complex by the TCR leads to T cell stimulation, which in turn leads to differentiation of both T helper cells (CD4+) and cytotoxic T lymphocytes (CD8+) in memory and effector lymphocytes. These cells then can expand in a clonal manner to give an activated subpopulation within the whole T cell population capable of reacting to one particular antigen.

MHC proteins are of two classes, I and II. The class I MHC proteins are heterodimers of two proteins, the α chain, which is a transmembrane protein encoded by the MHC1 class I genes, and the β2 microglobulin chain (sometimes referred to as B2M), which is a small extracellular protein that is encoded by a gene that does not lie within the MHC gene cluster. The α chain folds into three globular domains and when the β2 microglobulin chain is associated, the globular structure complex functional and expressed on the cell surface. Peptides are presented on the two most N-terminal domains which are also the most variable. Class II MHC proteins are also heterodimers, but the heterodimers comprise two transmembrane proteins encoded by genes within the MHC complex. The class I MHC:antigen complex interacts with cytotoxic T cells while the class II MHC presents antigens to helper T cells. In addition, class I MHC proteins tend to be expressed in nearly all nucleated cells and platelets (and red blood cells in mice) while class II MHC protein are more selectively expressed. Typically, class II MHC proteins are expressed on B cells, some macrophage and monocytes, Langerhans cells, and dendritic cells.

In humans, the major histocompatibility complex (MHC) is commonly known as the human leukocyte antigen (HLA). The class I HLA gene cluster in humans comprises three major loci, B, C and A, as well as several minor loci (including E, G and F, all found in the HLA region on chromosome 6). The class II HLA cluster also comprises three major loci, DP, DQ and DR, and both the class I and class II gene clusters are polymorphic, in that there are several different alleles of both the class I and II genes within the population. There are also several accessory proteins that play a role in HLA functioning as well. β-2 microglobulin functions as a chaperon (encoded by B2M, located on chromosome 15) and stabilizes the HLA A, B or C protein expressed on the cell surface and also stabilizes the antigen display groove on the class I structure. It is found in the serum and urine in low amounts normally.

HLA plays a major role in transplant rejection. The acute phase of transplant rejection can occur within about 1-3 weeks and usually involves the action of host T lymphocytes on donor tissues due to sensitization of the host system to the donor class I and class II HLA molecules. In most cases, the triggering antigens are the class I HLAs. For best success, donors are typed for HLA and matched to the patient recipient as completely as possible. But donation even between family members, which can share a high percentage of HLA identity, is still often not successful. Thus, in order to preserve the graft tissue within the recipient, the patient often must be subjected to profound immunosuppressive therapy to prevent rejection. Such therapy can lead to complications and significant morbidities due to opportunistic infections that the patient may have difficulty overcoming. Regulation of the class I or II genes can be disrupted in the presence of some tumors and such disruption can have consequences on the prognosis of the patients. For example, reduction of B2M expression was found in metastatic colorectal cancers (Shrout, et al. (2008) Br J Canc 98: 1999). Since B2M has a key role in stabilizing the MHC class I complex, loss of B2M in certain solid cancers has been hypothesized to be a mechanism of immune escape from T cell driven immune surveillance. Depressed B2M expression has been shown to be a result of suppression of the normal IFN gamma B2M expressional regulation and/or specific mutations in the B2M coding sequence that result in gene knock-out (Shrout, et al., ibid). Confoundingly, increased B2M is also associated with some types of cancer. Increased B2M levels in the urine serves as a prognosticator for several cancers including prostate, chronic lymphocytic leukemia (CLL) and Non-Hodgkin’s lymphomas.

Adoptive cell therapy (ACT) is a developing form of cancer therapy based on delivering tumor-specific immune cells to a patient in order for the delivered cells to attack and clear the patient’s cancer. ACT can involve the use of tumor-infiltrating lymphocytes (TILs) which are T-cells that are isolated from a patient’s own tumor masses and expanded ex vivo to re-infuse back into the patient. This approach has been promising in treating metastatic melanoma, where in one study, a long term response rate of >50% was observed (see for example, Rosenberg, et al. (2011) Clin Canc Res 17(13): 4550). TILs are a promising source of cells because they are a mixed set of the patient’s own cells that have T-cell receptors (TCRs) specific for the Tumor associated antigens (TAAs) present on the tumor (Wu, et al. (2012) Cancer J 18(2):160). Other approaches involve editing T cells isolated from a patient’s blood such that they are engineered to be responsive to a tumor in some way (Kalos, et al. (2011) Sci TranslMed 3(95):95ra73).

Chimeric Antigen Receptors (CARs) are molecules designed to target immune cells to specific molecular targets expressed on cell surfaces. In their most basic form, they are receptors introduced into a cell that couple a specificity domain expressed on the outside of the cell to signaling pathways on the inside of the cell such that when the specificity domain interacts with its target, the cell becomes activated. Often CARs are made from emulating the functional domains of T-cell receptors (TCRs) where an antigen specific domain, such as a scFv or some type of receptor, is fused to the signaling domain, such as ITAMs and other co-stimulatory domains. These constructs are then introduced into a T-cell ex vivo allowing the T-cell to become activated in the presence of a cell expressing the target antigen, resulting in the attack on the targeted cell by the activated T-cell in a non-MHC dependent manner (see Chicaybam, et al. (2011) Int Rev Immunol 30:294-311) when the T-cell is re-introduced into the patient. Thus, adoptive cell therapy using T cells altered ex vivo with an engineered TCR or CAR is a very promising clinical approach for several types of diseases. For example, cancers and their antigens that are being targeted includes follicular lymphoma (CD20 or GD2), neuroblastoma (CD 171), non-Hodgkin lymphoma (CD 19 and CD20), lymphoma (CD19), glioblastoma (IL13Rα2), chronic lymphocytic leukemia or CLL and acute lymphocytic leukemia or ALL (both CD19). Virus specific CARs have also been developed to attack cells harboring virus such as HIV. For example, a clinical trial was initiated using a CAR specific for Gp100 for treatment of HIV (Chicaybam, ibid).

ACTRs (Antibody-coupled T-cell Receptors) are engineered T cell components that are capable of binding to an exogenously supplied antibody. The binding of the antibody to the ACTR component arms the T cell to interact with the antigen recognized by the antibody, and when that antigen is encountered, the ACTR comprising T cell is triggered to interact with antigen (see U.S. Pat. Publication No. 2015/0139943).

One of the drawbacks of adoptive cell therapy however is the source of the cell product must be patient specific (autologous) to avoid potential rejection of the transplanted cells. This has led researchers to develop methods of editing a patient’s own T cells to avoid this rejection. For example, a patient’s T cells or hematopoietic stem cells can be manipulated ex vivo with the addition of an engineered CAR, ACTR and/or T cell receptor (TCR), and then further treated with engineered nucleases to knock out T cell check point inhibitors such as PD1 and/or CTLA4 (see International Patent Publication No. WO 2014/059173). For application of this technology to a larger patient population, it would be advantageous to develop a universal population of cells (allogeneic). In addition, knockout of the TCR will result in cells that are unable to mount a graft-versus-host disease (GVHD) response once introduced into a patient.

Thus, there remains a need for methods and compositions that can be used to modify (e.g., knock out) TCR and/or HLA expression in effector T cells, regulatory T cells, B cells, NK cells or stem cells (e.g., hematopoietic stem cells, induced pluripotent stem cells and embryonic stem cells).

### SUMMARY

Disclosed herein are compositions and methods for partial or complete inactivation or disruption of a TCR and/or B2M gene and compositions and methods for introducing and expressing to desired levels of exogenous transgenes in T lymphocytes, after or simultaneously with the disruption of the endogenous TCR and/or B2M. Also provided herein are methods and compositions for deleting (inactivating) or repressing a TCR and/or B2M gene to produce TCR null T cell or TCR and HLA class I null T cell, B cells, NK cell, stem cell, tissue or whole organism, for example a cell that does not express one or more T cell receptors and/or one or more HLA class I receptors on its surface. Additional genomic modifications may be present in the TCR and/or HLA class I null cells described herein, including, but not limited to genomic modifications to a different gene (e.g., a programmed cell death 1 (PD1) gene, a Cytotoxic T-Lymphocyte Antigen 4 (CTLA-4) gene, a CISH gene, a tet2 gene, an human leukocyte antigen (HLA) A gene, an HLA B gene, an HLA C gene, an HLA-DPA gene, an HLA-DQ gene, an HLA-DRA gene, a LMP7 gene, a Transporter associated with Antigen Processing (TAP) 1 gene, a TAP2 gene, a tapasin gene (TAPBP), a class II major histocompatibility complex transactivator (CIITA) gene, a glucocorticoid receptor gene (GR), an IL2RG gene, an RFX5 gene), insertion of transgene (e.g., CAR) into one or more of these or other genes (e.g., safe harbor genes) and any combination of such genomic modifications. In certain embodiments, the TCR null cells and/or HLA class I null cells, or tissues are human cells or tissues that are advantageous for use in transplants. In preferred embodiments, the TCR null T cells and/or HLA class I null cells are prepared for use in adoptive T cell therapy.

In one aspect, described herein is a zinc finger nuclease comprising: a ZFP from a ZFN designated 68957, 72678, 72732 or 72748; an engineered FokI cleavage domain; and a linker between the FokI cleavage domain and the ZFP. In certain embodiments, the ZFN comprises first and second ZFNs as follows (amino acid and polynucleotide sequences disclosed in the Examples): a ZFN comprising a ZFP from the ZFN designated 72678 and a ZFN comprising a ZFP from the ZFN designated 72732. In certain embodiments the ZFN comprises left and right (first and second) ZFNs as follows: a ZFN designated 57531 and a ZFN designated 72732; a ZFN designated 57531 and a ZFN designated 72748; a ZFN designated 68957 and a ZFN designated 57071; a ZFN designated 68957 and a ZFN designated 72732; a ZFN designated 68957 and a ZFN designated 72748; a ZFN designated 72678 and a ZFN designated 57071; a ZFN designated 72678 and a ZFN designated 72732; and a comprising a ZFP ZFN designated 72678 and a ZFN designated 72748. A zinc finger nuclease (ZFN) comprising left and right (first and second) ZFNs as follows: a ZFN designated 68796 and a ZFN designated 68813; a ZFN designated 68796 and a ZFN designated 68861; a ZFN designated 68812 and a ZFN designated 68813; a ZFN designated 68876 and a ZFN designated 68877; a ZFN designated 68815 and a ZFN designated 55266; a ZFN designated 68879 and a ZFN designated 55266; a ZFN designated 68798 and a ZFN designated 68815; or a ZFN designated 68846 and a ZFN designated 53853. Polynucleotides (e.g., mRNA, plasmids, viral vectors, etc.) encoding a ZFN (including a pair) as disclosed herein are also provided, including a polynucleotide comprising a 2A sequence between the sequences encoding the left and ZFNs. Also disclosed are genetically modified cells (e.g., stem cells, precursor cells, T cells (effector and regulatory), etc.) comprising one or more of the ZFNs and/or polynucleotides disclosed herein and cells descended from these cells (e.g., genetically modified cells that do not comprise the ZFN but include the genetic modification). The genetic modifications include insertions, deletions and combinations thereof in the gene targeted by the ZFN. Additional genomic modifications, for example, modification of a T cell receptor (TCR) gene, modification of an HLA-A gene, modification of an HLA-B gene, modification of an HLA-C gene, modification of a TAP gene, modification of a CTLA-4 gene, modification of a PD1 gene, modification of a CISH gene, modification of a tet-2 gene, and/or insertion of a transgene (e.g., CAR) may be present at the target and/or one or more different loci. Pharmaceutical compositions comprising any of the zinc finger nucleases, polynucleotides, and/or cells as described herein are also provided. Methods of modifying an endogenous beta-2-microglobulin (B2M) and/or TCR gene in a cell are also provided, the method comprising administering a polynucleotide or pharmaceutical composition as described herein to the cell such that the endogenous gene is modified (e.g., deletion, insertion of an exogenous sequence such as a transgene). Methods of using the ZFNs, polynucleotides, cells and/or pharmaceutical compositions as described herein for the treatment and/or prevention of a cancer, an autoimmune disease or graft-versus-host disease are also provided. Kits comprising any of the ZFNs, polynucleotides, cells and/or pharmaceutical compositions as described herein are also provided.

In other aspects, described herein is an isolated cell (e.g., a eukaryotic cell such as a mammalian cell including a lymphoid cell, a stem cell (e.g., iPSC, embryonic stem cell, MSC or HSC), or a progenitor/precursor cell) in which expression of a TCR gene is modulated by modification of exonic sequences of the TCR gene. In certain embodiments, the modification is to a sequence comprising a sequence of 9-25 (including target sites of 9, 10, 11, 12, 13, 14, 15, 16, 17, 18, 19, 20, 21, 22, 23, 24, 25) or more nucleotides (contiguous or non-contiguous) of a sequence as shown in the target sites herein) of a target site as shown in one or more of Tables 1, 2 or 6 (SEQ ID NO: 8-21 and/or 92-103); within 1-5, within 1-10 or within 1-20 base pairs on either side (the flanking genomic sequence) of the target sites shown in Tables 1, 2 or 6(SEQ ID NO:8-21 and/or 92-103); or within AACAGT, AGTGCT, CTCCT, TTGAAA, TGGACTT and AATCCTC or a target site comprising AACAGT, AGTGCT, CTCCT, TTGAAA, TGGACTT and AATCCTC. Alternatively, or in addition, the modifications may also be made to sequences (e.g., genomic sequences) between paired target sites of as described herein (e.g., target sites for the nuclease pairs shown in Table 3, including between the target sites for 55204 and 53759 (between SEQ ID NO:8 and SEQ ID NO:9); between the target sites for 55229 and 53785 (between SEQ ID NO:10 and SEQ ID NO:11); between the target sites for 53810 and 55255 (between SEQ ID NO:12 and SEQ ID NO:13); between the target sites shown for 55248 and 55254/55260 (between SEQ ID NO:14 and SEQ ID NO:13); between the target sites for 55266 and 53853 (between SEQ ID NO:15 and SEQ ID NO:16); between the target sites for 53860 and 53863 (between SEQ ID NO:17 and SEQ ID NO:18); between the target sites for 53856 and 55287 (between SEQ ID NO:21 and SEQ ID NO:18); or between the target sites for 53885 or 52774 and 53909 or 52742 (between SEQ ID NO:19 and SEQ ID NO:20). The modification may be by an exogenous fusion molecule comprising a functional domain (e.g., transcriptional regulatory domain, nuclease domain including any FokI cleavage domain with one or more mutations as compared to wild-type) and a DNA-binding domain, including, but not limited to: (i) a cell comprising an exogenous transcription factor comprising a DNA-binding domain that binds to a target site as shown in any of SEQ ID NO:8-21 and/or 92-103 and a transcriptional regulatory domain in which the transcription factor modifies TRAC gene expression and/or (ii) a cell comprising an insertion and/or a deletion within one or more of the target sites shown herein, including SEQ ID NO:8-21 and/or 92-103; within 1-5, within 1-10 or within 1-20 base pairs on either side (the flanking genomic sequence) of the target sites shown in Tables 1 and 2 (SEQ ID NO: 8-21 and/or 92-103); within AACAGT, AGTGCT, CTCCT, TTGAAA, TGGACTT and AATCCTC; and/or between paired target sites as described herein (e.g., target sites for the nuclease pairs shown in Table 3). Cells comprising these modifications to TCR gene(s) and additional genetic modifications (e.g., B2M gene modification, CTLA, CISH, PD1 and/or tet2 gene modifications, CAR, an antigen-specific TCR (alpha and beta chains), insertions at these or other loci including a transgene encoding an Antibody-coupled T-cell Receptor (ACTR) and/or a transgene encoding an antibody, etc.) are also described.

In another aspect, described herein is an isolated cell (e.g., a eukaryotic cell such as a mammalian cell including a lymphoid cell, a stem cell (e.g., iPSC, embryonic stem cell, MSC or HSC), or a progenitor/precursor cell) in which expression of a B2M gene is modulated by modification of the B2M gene. In certain embodiments, the modification is to a sequence comprising a sequence of 9-25 (including target sites of 9, 10, 11, 12, 13, 14, 15, 16, 17, 18, 19, 20, 21, 22, 23, 24, 25) or more nucleotides (contiguous or non-contiguous) of a sequence as shown in the target sites herein) of a target site as shown in one or more of Tables 5 and 8 (SEQ ID NO: 117, 123, 126 and/or 127); within 1-5, within 1-10 or within 1-20 base pairs on either side (the flanking genomic sequence) of the target sites shown in Tables 5 and 8 (SEQ ID NO:117, 123, 126 and/or 127). Alternatively, or in addition, the modifications may also be made to sequences (e.g., genomic sequences) between paired target sites of as described herein (e.g., target sites for the nuclease pairs shown in Tables 5 and 8, including between the target sites as shown in Table 8 (SEQ ID NO:126 and 127). The modification may be by an exogenous fusion molecule comprising a functional domain (e.g., transcriptional regulatory domain, nuclease domain including any FokI cleavage domain with one or more mutations as compared to wild-type) and a DNA-binding domain (e.g., a ZFP as shown in Table 8 (the ZFP component (designs) of the ZFNs designated 72732; 72748; 68957; or 72678), including, but not limited to: (i) a cell comprising an exogenous transcription factor comprising a DNA-binding domain that binds to a target site as shown in any of Tables 5 or 8 (e.g., SEQ ID NO: 126 or 127) and a transcriptional regulatory domain in which the transcription factor modifies B2M gene expression and/or (ii) a cell comprising an insertion and/or a deletion within one or more of the target sites shown herein, including Tables 5 and 8; within 1-5, within 1-10 or within 1-20 base pairs on either side (the flanking genomic sequence); and/or between paired target sites as described herein (e.g., target sites for the nuclease pairs shown in Table 8). Cells comprising these modifications to B2M genes and additional genetic modifications (e.g., TCR gene modification, CTLA, CISH, PD1 and/or tet2 gene modifications, PD1 modification, a CAR insertion, an antigen-specific TCR (alpha and beta chains), insertions at these or other loci including a transgene encoding an Antibody-coupled T-cell Receptor (ACTR) and/or a transgene encoding an antibody, etc.) are also described.

The TCR and/or B2M modified cells described herein may include further modifications, for example one or more inactivated T-cell receptor genes in B2M modified cells, additional inactivated TCR genes, PD1 and/or CTLA4 gene and/or a transgene a transgene encoding a chimeric antigen receptor (CAR), a transgene encoding an Antibody-coupled T-cell Receptor (ACTR) and/or a transgene encoding an antibody. Pharmaceutical compositions comprising any cell as described herein are also provided as well as methods of using the cells and pharmaceutical compositions in ex vivo therapies for the treatment of a disorder (e.g., a cancer) in a subject. In certain embodiments, a population of cells comprising one or more modifications (TCR edits, B2M edits, PD1 edits, CISH, tet2 and/or CTLA4 edits, HLA class I gene edits and/or transgene (e.g., CAR) insertions into these or other genes, etc.) as described herein are provided, including a population of cells in which less than 5% (e.g., 0-5% or any value therebetween), preferably less than 3%, even more preferably less than 2% of the cells include any other modifications (e.g., modifications at off-target sites). In certain embodiments, the population of cells includes modifications at off-target sites at background levels (e.g., 2-10-fold less (or any value therebetween)) as compared to cells modified with ZFNs that are not modified as described herein (which unmodified ZFNs are also referred to as “parent” or “parental” ZFNs). The modifications made by the ZFNs are heritable in that, in vivo or in culture, cells descended from (including differentiated cells) cells comprising the ZFNs (and modifications) include the modifications described herein.

Thus, in one aspect, described herein are cells in which the expression of a TCR gene is modulated (e.g., activated, repressed or inactivated). In preferred embodiments, exonic sequences of a TCR gene are modulated. The modulation may be by an exogenous molecule (e.g., engineered transcription factor comprising a DNA-binding domain and a transcriptional activation or repression domain) that binds to the TCR gene and regulates TCR expression and/or via sequence modification of the TCR gene (e.g., using a nuclease that cleaves the TCR gene and modifies the gene sequence by insertions and/or deletions), including for example a ZFN (e.g., ZFN pair of left and right ZFNs) as shown in Table 6. In some embodiments, cells are described that comprise an engineered nuclease to cause a knockout of a TCR gene. In other embodiments, cells are described that comprise an engineered transcription factor (TF) such that the expression of a TCR gene is modulated. In some embodiments, the cells are T cells. Further described are cells wherein the expression of a TCR gene is modulated and wherein the cells are further engineered to comprise a least one exogenous transgene and/or an additional knock out of at least one endogenous gene (e.g., beta 2 microglobuin (B2M) and/or immunological checkpoint gene such as PD1 and/or CTLA4) or combinations thereof.

In another aspect, described herein are cells in which the expression of a B2M gene is modulated (e.g., activated, repressed or inactivated). The modulation may be by an exogenous molecule (e.g., engineered transcription factor comprising a DNA-binding domain and a transcriptional activation or repression domain) that binds to the B2M gene and regulates B2M expression and/or via sequence modification of the B2M gene (e.g., using a nuclease that cleaves the B2M gene and modifies the gene sequence by insertions and/or deletions), including for example a ZFN (e.g., ZFN pair of left and right ZFNs) as shown in Table 8 or a ZFN comprising a ZFP having the design (recognition helix region and backbone of ZFPs in ZFNs designated 72732; 72748; 68957; or 72678) described herein (e.g., Table 8) in combination with any FokI domain (wild-type or engineered) and optionally any linker between the FokI domain and the ZFP (e.g., L0, N7a, N7c, etc.). In some embodiments, cells are described that comprise an engineered nuclease to cause a knockout of a B2M gene. In other embodiments, cells are described that comprise an engineered transcription factor (TF) such that the expression of a B2M gene is modulated. In some embodiments, the cells are T cells, including effector T cells and regulatory T cells. Further described are cells wherein the expression of a B2M gene is modulated and wherein the cells are further engineered to comprise a least one exogenous transgene and/or an additional knock out of at least one endogenous gene (e.g., one or more TCR genes and/or immunological checkpoint gene such as PD1 and/or CTLA4) or combinations thereof.

In any of the cells described herein comprising an exogenous transgene, the exogenous transgene may be integrated into a TCR and/or B2M gene (e.g., when the TCR and/or B2M gene is knocked out) and/or may be integrated into a gene such as a safe harbor gene. In some cases, the exogenous transgene encodes an ACTR, an antigen-specific TCR, and/or a CAR. The transgene construct may be inserted by either HDR- or NHEJ- driven processes. In some aspects the cells with modulated TCR and/or B2M expression comprise at least an exogenous ACTR, an exogenous TCR and an exogenous CAR. Some cells comprising a TCR modulator further comprise a knockout of one or more check point inhibitor genes. In some embodiments, the check point inhibitor is PD1. In other embodiments, the check point inhibitor is CTLA4. In further aspects, the TCR and/or B2M modulated cell comprises a PD1 knockout and a CTLA4 knockout. In some embodiments, the TCR gene modulated is a gene encoding TCR β (TCRB). In some embodiments this is achieved via targeted cleavage of the constant region of this gene (TCR β Constant region, or TRBC). In certain embodiments, the TCR gene modulated is a gene encoding TCR α (TCRA). In further embodiments, insertion is achieved via targeted cleavage of the constant region of a TCR gene, including targeted cleavage of the constant region of a TCR α gene (referred to herein as “TRAC” sequences). In some embodiments, the TCR gene modified cells are further modified at the B2M gene, the HLA-A, -B, -C genes, or the TAP gene, or any combination thereof. In other embodiments, the regulator for HLA class II, CIITA, is also modified.

In certain embodiments, the cells described herein comprise a modification (e.g., deletion and/or insertion, binding of an engineered TF to repress TCR expression) to a TCRA gene (e.g., modification of exons). In certain embodiments, the modification is within any of the target sites shown in Tables 1, 2 or 6 (SEQ ID NO:8-21 and/or 92-103) and/or between paired target sites (e.g., target sites of nuclease pairs shown in Table 3), including modification by binding to, cleaving, inserting and/or deleting one or more nucleotides within any of these sequences and/or within 1-50 base pairs (including any value therebetween such as 1-5, 1-10 or 1-20 base pairs) of the gene (genomic) sequences flanking these sequences in the TCRA gene. In certain embodiments, the modifications are made using a ZFN (e.g., one or more ZFN pairs) as shown in Table 6. In certain embodiments, the cells comprise a modification (binding to, cleaving, insertions and/or deletions) within one or more of the following sequences: AACAGT, AGTGCT, CTCCT, TTGAAA, TGGACTT and AATCCTC within a TCRA gene (e.g., exons, see FIG. 1B). In certain embodiments, the modification comprises binding of an engineered TF as described herein such that a TCRA gene expression is modulated, for example, repressed or activated.

In certain embodiments, the cells described herein comprise a modification (e.g., deletion and/or insertion, binding of an engineered TF to repress B2M expression) to a B2M gene. In certain embodiments, the modification is within any of the target sites shown in Tables 5 or 8 and/or between paired target sites (e.g., target sites of nuclease pairs shown in Table 8), including modification by binding to, cleaving, inserting and/or deleting one or more nucleotides within any of these sequences and/or within 1-50 base pairs (including any value therebetween such as 1-5, 1-10 or 1-20 base pairs) of the gene (genomic) sequences flanking these sequences in the B2M gene. In certain embodiments, the modifications are made using a ZFN comprising a ZFP comprising the recognition helix regions and backbone of the ZFP designs of the ZFNs shown in Table 8, a FokI domain (any wild-type or engineered FokI domain) and optionally a linker (any linker between the N- or C-terminal of the FokI domain and the N- or C-terminal of the ZFP designs shown including but not limited to L0, N7a, N7c, etc.). In certain embodiments, the ZFN comprises a ZFN (e.g., a pair of first and second ZFNs) as shown in Table 8. In certain embodiments, the cells comprise a modification (binding to, cleaving, insertions and/or deletions) within one or more of the following sequences: SEQ ID NO: 126 and 127. In certain embodiments, the modification comprises binding of an engineered TF as described herein such that B2M gene expression is modulated, for example, repressed or activated.

In other embodiments, the modification is a genetic modification (alteration of nucleotide sequence) at or near nuclease(s) binding (target) and/or cleavage site(s), including but not limited to, modifications to sequences within 1-300 (or any number of base pairs therebetween) base pairs upstream, downstream and/or including 1 or more base pairs of the site(s) of cleavage and/or binding site; modifications within 1-100 base pairs (or any number of base pairs therebetween) of including and/or on either side of the binding and/or cleavage site(s); modifications within 1 to 50 base pairs (or any number of base pairs therebetween) including and/or on either side (e.g., 1 to 5, 1 to 10, 1 to 20 or more base pairs) of the binding and/or cleavage site(s); and/or modifications to one or more base pairs within the nuclease binding site and/or cleavage site. In certain embodiments, the modification is at or near (e.g., 1-300 base pairs, 1-50, 1-20, 1-10 or 1-5 or any number of base pairs therebetween) and/or between paired target sites (e.g., Table 3 or 8) of the gene sequence surrounding or between any of the target sites disclosed herein. In certain embodiments, the modification includes modifications of a TCRA and/or B2M gene within one or more of the sequences shown in in the target sites of Tables 1, 2 and 6 (TCRA) and/or Tables 5 and 8 (B2M), for example a modification of 1 or more base pairs to one or more of these sequences. In certain embodiments, the nuclease-mediated genetic modifications are between paired target sites (when a dimer is used to cleave the target). The nuclease-mediated genetic modifications may include insertions and/or deletions of any number of base pairs, including insertions of non-coding sequences of any length and/or transgenes of any length and/or deletions of 1 base pair to over 1000 kb (or any value therebetween including, but not limited to, 1-100 base pairs, 1-50 base pairs, 1-30 base pairs, 1-20 base pairs, 1-10 base pairs or 1-5 base pairs).

The modified cells of the invention may be a eukaryotic cell, including a non-human mammalian and a human cell such as lymphoid cell (e.g., a T-cell (including an effector T cell (Teff) and a regulatory T cell (Treg)), a B cell or an NK cell), a stem/progenitor cell (e.g., an induced pluripotent stem cell (iPSC), an embryonic stem cell (e.g., human ES), a mesenchymal stem cell (MSC), or a hematopoietic stem cell (HSC). The stem cells may be totipotent or pluripotent (e.g., partially differentiated such as an HSC that is a pluripotent myeloid or lymphoid stem cell). In other embodiments, the invention provides methods for producing cells that have a null genotype for TCR and or HLA expression. Any of the modified stem cells described herein (modified at the TCRA and/or B2M loci) may then be differentiated to generate a differentiated (in vivo or in vitro (culture)) cell descended from a stem cell as described herein with the modifications described herein, including modified TCRA and/or B2M gene expression.

In another aspect, the compositions (modified cells) and methods described herein can be used, for example, in the treatment or prevention or amelioration of a disorder. The methods typically comprise (a) cleaving or down regulating an endogenous TCR and/or B2M gene in an isolated cell (e.g., T-cell or other lymphocytes) using a nuclease (e.g., ZFN or TALEN) or nuclease system such as CRISPR/Cas with an engineered crRNA/tracr RNA, or using an engineered transcription factor (e.g., ZFP-TF, TALE-TF, Cfp1-TF or Cas9-TF) such that the TCR and/or B2M gene is inactivated or down modulated; and (b) introducing the cell into the subject, thereby treating or preventing the disorder. In some embodiments, the gene encoding TCR β (TCRB) is inactivated or down-modulated. In some embodiments, the gene encoding B2M is inactivated or down-modulated. In some embodiments inactivation is achieved via targeted cleavage of the constant region of this gene (TCR β Constant region, or TRBC). In preferred embodiments, the gene encoding TCR α (TCRA) and/or B2M is inactivated or down modulated. In further preferred embodiments, the disorder is a cancer, an infectious disease or an autoimmune disease. In some embodiments, the modifications are made to induce immune tolerance. In further preferred embodiments inactivation is achieved via targeted cleavage of the constant region of this gene (TCR α Constant region, or abbreviated as TRAC). In some embodiments, a B2M gene is cleaved. In further embodiments, the additional genes (in addition to TCR and/or B2M) are modulated (knocked-out), for example, TCR/B2M double knockouts, additional TCR genes, PD1 and/or CTLA4 and/or one or more therapeutic transgenes are present in the cell (episomal, randomly integrated or integrated via targeted integration such as nuclease-mediated integration). The modified cells may include one or more ZFNs (e.g., ZFN pairs) as described herein, including but not limited to a zinc finger nuclease (ZFN) comprising first and second ZFNs, each ZFN comprising a cleavage domain (e.g., any wild-type or engineered FokI cleavage domain) and a ZFP DNA-binding domain. In certain embodiments, the modifications are made using a ZFN comprising a ZFP (recognition helix regions and backbone) of the “designs” described herein (e.g., Table 6 or Table 8 including the ZFPs of the ZFNs designated 68846, 53853, 72732; 72748; 68957; 55266, 68798, 68879, 68815, 68799 or 72678), a FokI domain (any wild-type or engineered FokI domain) and optionally a linker (any linker between the N- or C-terminal of the FokI domain and the N- or C-terminal of the ZFP designs described herein). In some embodiments the ZFN comprises a pair of ZFNs, in which one ZFN comprises the ZFP of 68846 (SEQ ID NO:177) operably linked to a FokI domain and the other ZFN of the pair comprises the ZFP of 53853 (SEQ ID NO:178) operably linked to a FokI domain. In some embodiments the ZFN comprises a pair of ZFNs, in which one ZFN comprises the ZFP of 72732 (SEQ ID NO: 175) operably linked to a FokI domain and the other ZFN of the pair comprises the ZFP of 72678 (SEQ ID NO:176) operably linked to a FokI domain. In certain embodiments, the ZFN comprises a ZFN (e.g., a pair of first and second (also referred to as left and right) partner ZFNs) described herein as follows: a ZFN designated 68796 and a ZFN designated 68813; a ZFN designated 68796 and a ZFN designated 68861; a ZFN designated 68812 and a ZFN designated 68813; a ZFN designated 68876 and a ZFN designated 68877; a ZFN designated 68815 and a ZFN designated 55266; a ZFN designated 68879 and a ZFN designated 55266; a ZFN designated 68798 and a ZFN designated 68815; or a ZFN designated 68846 and a ZFN designated 53853; a ZFN designated 57531 and a ZFN designated 72732; a ZFN designated 57531 and a ZFN designated 72748; a ZFN designated 68957 and a ZFN designated 57071; a ZFN designated 68957 and a ZFN designated 72732; a ZFN designated 68957 and a ZFN designated 72748; a ZFN designated 72678 and a ZFN designated 57071; a ZFN designated 72678 and a ZFN designated 72732; and a comprising a ZFP ZFN designated 72678 and a ZFN designated 72748. Thus, a ZFN (e.g., each ZFN partner of a paired ZFN) comprises the recognition helix regions and may comprise additional ZFP modifications (e.g., to the backbone regions) described below (e.g., designs shown in Tables 1, 2, 5, 6 and 8) and further comprises any wild-type or engineered FokI cleavage domain (including any combination of the FokI substitution, addition and/or deletion mutants). For example, a ZFN partner may comprise specific zinc finger DNA binding domain fused to any FokI cleavage domain including the cleavage domain (SEQ ID NO: 139) from the wildtype protein or from a mutated sequence (as shown in the Examples, SEQ ID NO: 140-174). A B2M-specific ZFN partner may comprise a B2M-specific zinc finger DNA binding domain (e.g., 72732) fused with a FokI cleavage domain selected from SEQ ID NOs: 139-174. Further, the B2M-specific ZFN partner may comprise a B2M-specific zinc finger DNA binding domain (e.g., 72678) fused to a FokI cleavage domain selected from SEQ ID NOs: 139-174. Similarly, a TRAC-specific ZFN partner may comprise a TRAC-specific zinc finger DNA binding domain (e.g., 68846) fused to a FokI cleavage domain selected from SEQ ID NOs: 139-174, and the TRAC-specific zinc finger DNA binding domain 53853 may be fused to a FokI cleavage domain selected from any of wild-type or engineered FokI cleavage shown, for example a domain as shown in the appended Examples (SEQ ID NOs: 139-174). In some embodiments, the FokI domain is fused at the N-terminal end of the ZFP DNA binding domain while in others, it is fused to the C-terminal end of the ZFP DNA binding domain. Further, any linker can be used to link the DNA-binding domain to the FokI cleavage domain.

Cells descended from cells modified as described herein (e.g., cells comprising the ZFNs described herein), including but not limited partially or fully differentiated from stem cells modified as described herein, are also provided. These cells typically do not include the ZFNs but do include the genetic modifications made thereby.

The transcription factor(s) and/or nuclease(s) can be introduced into a cell or the surrounding culture media as mRNA, in protein form and/or as a DNA sequence encoding the nuclease(s). In certain embodiments, the isolated cell introduced into the subject further comprises additional genomic modification, for example, an integrated exogenous sequence (into the cleaved TCR and/or B2M gene or a different gene, for example a safe harbor gene or locus) and/or inactivation (e.g., nuclease-mediated) of additional genes, for example one or more HLA genes, or CTLA-4, CISH, PD1, or tet2 genes. The exogenous sequence (e.g., a CAR or exogenous TCR) or protein may be introduced via a vector (e.g., Ad, AAV, LV), or by using a technique such as electroporation or transient transfection. In some embodiments, the proteins are introduced into the cell by inducing mechanical stress such as cell squeezing (see Kollmannsperger, et al. (2016) Nat Comm 7, 10372 doi:10.1038/ncomms10372). In some aspects, the composition may comprise isolated cell fragments and/or differentiated (partially or fully) cells.

In some aspects, the modified cells may be used for cell therapy, for example, for adoptive cell transfer. In other embodiments, the cells for use in T cell transplant contain another gene modification of interest. In one aspect, the T cells contain an inserted chimeric antigen receptor (CAR) specific for a marker found on cancer cells. In a further aspect, the inserted CAR is specific for the CD19 marker characteristic of B cells, including B cell malignancies. Such cells would be useful in a therapeutic composition for treating patients without having to match HLA, and so would be able to be used as an “off-the-shelf” therapeutic for any patient in need thereof. In other instances, stem or precursor cells, for example, hematopoietic stem cell or precursor cells (HSC/PC) or induced pluripotent stem cells (iPSC) containing the modifications described herein are expanded prior to introduction. In other aspects, the genetically modified HSC/PCs are given to the subject in a bone marrow transplant wherein the HSC/PC engraft, differentiate and mature in vivo. In some embodiments, the HSC/PC are isolated from the subject following G-CSF-induced mobilization, plerixafor-induced mobilization, and combinations of G-CSF- and plerixafor-induced mobilization, and in others, the cells are isolated from human bone marrow or human umbilical cords. In other embodiments, iPSC are derived from patient or healthy donor cells. In some aspects, the subject is treated to a mild myeloablative procedure prior to introduction of the graft comprising the modified HSC/PC or modified cells derived from iPSC, while in other aspects, the subject is treated with a vigorous myeloablative conditioning regimen. In some embodiments, the methods and compositions of the invention are used to treat or prevent a cancer.

In another aspect, the TCR- and/or B2M-modulated (modified) T cells contain an inserted Antibody-coupled T-cell Receptor (ACTR) donor sequence. In some embodiments, the ACTR donor sequence is inserted into a TCR gene to disrupt expression of that TCR gene following nuclease induced cleavage. In other embodiments, the donor sequence is inserted into a “safe harbor” locus, such as the AAVS1, HPRT, albumin and CCR5 genes. In some embodiments, the ACTR sequence is inserted via targeted integration where the ACTR donor sequence comprises flanking homology arms that have homology to the sequence flanking the cleavage site of the engineered nuclease. In some embodiments the ACTR donor sequence further comprises a promoter and/or other transcriptional regulatory sequences. In other embodiments, the ACTR donor sequence lacks a promoter. In some embodiments, the ACTR donor is inserted into a TCR β encoding gene (TCRB). In some embodiments insertion is achieved via targeted cleavage of the constant region of this gene (TCR β Constant region, or TRBC). In preferred embodiments, the ACTR donor is inserted into a TCR α encoding gene (TCRA). In further preferred embodiments insertion is achieved via targeted cleavage of the constant region of this gene (TCR α Constant region, abbreviated TRAC). In some embodiments, the donor is inserted into an exon sequence in TCRA, while in others, the donor is inserted into an intronic sequence in TCRA. In still further embodiments, the ACTR donor is inserted into a B2M gene. In some embodiments, the B2M and/or TCR-modulated cells further comprise a CAR. In still further embodiments, the B2M and/or TCR-modulated cells are additionally modulated at an HLA gene or a checkpoint inhibitor gene.

Also provided are pharmaceutical compositions comprising the modified cells as described herein (e.g., T cells or stem cells with inactivated TCR gene), or pharmaceutical compositions comprising one or more of the TCR and/or B2M gene binding molecules (e.g., engineered transcription factors and/or nucleases) as described herein. In certain embodiments, the pharmaceutical compositions further comprise one or more pharmaceutically acceptable excipients. The modified cells, TCR and/or B2M gene binding molecules (or polynucleotides encoding these molecules) and/or pharmaceutical compositions comprising these cells or molecules are introduced into the subject via methods known in the art, e.g., through intravenous infusion, infusion into a specific vessel such as the hepatic artery, or through direct tissue injection (e.g., muscle). In some embodiments, the subject is an adult human with a disease or condition that can be treated or ameliorated with the composition. In other embodiments, the subject is a pediatric subject where the composition is administered to prevent, treat or ameliorate the disease or condition (e.g., cancer, graft versus host disease, etc.).

In some aspects, the composition (TCR and/or B2M modulated cells comprising an ACTR) further comprises an exogenous antibody. See, also, U.S. Pat. Publication No. 2017/0196992. In some aspects, the antibody is useful for arming an ACTR-comprising T cell to prevent or treat a condition. In some embodiments, the antibody recognizes an antigen associated with a tumor cell or with cancer associate processes such as EpCAM, CEA, gpA33, mucins, TAG-72, CAIX, PSMA, folate-binding antibodies, CD19, EGFR, ERBB2, ERBB3, MET, IGF1R, EPHA3, TRAILR1, TRAILR2, RANKL, FAP, VEGF, VEGFR, αVβ3 and α5β1 integrins, CD20, CD30, CD33, CD52, CTLA4, and enascin (Scott, et al. (2012) Nat Rev Cancer 12:278). In other embodiments, the antibody recognizes an antigen associated with an infectious disease such as HIV, HCV and the like.

In another aspect, provided herein are TCR gene DNA-binding domains (e.g., ZFPs, TALEs and sgRNAs) that bind to a target site in a TCR gene. In certain embodiments, the DNA binding domain comprises a ZFP with the recognition helix regions in the order as shown in a single row of Table 1; a TAL-effector domain DNA-binding protein with the RVDs that bind to a target site as shown in the first column of Table 1 or the third column of Table 2; and/or a sgRNA as shown in a single row of Table 2. These DNA-binding proteins can be associated with transcriptional regulatory domains to form engineered transcription factors that modulate TCR expression. Alternatively, these DNA-binding proteins can be associated with one or more nuclease domains to form engineered zinc finger nucleases (ZFNs), TALENs and/or CRISPR/Cas systems that bind to and cleave a TCR gene. In certain embodiments, the ZFNs, TALENs or single guide RNAs (sgRNA) of a CRISPR/Cas system bind to target sites in a human TCR gene. The DNA-binding domain of the transcription factor or nuclease (e.g., ZFP, TALE, sgRNA) may bind to a target site in a TCRA gene comprising 9, 10, 11, 12 or more (e.g., 13, 14, 15, 16, 17, 18, 19, 20 or more) nucleotides of any of the target sites shown herein (e.g., target sites of Table 1 or 2 as shown in SEQ ID NOs:8-21 and/or 92-103). The zinc finger proteins may include 1, 2, 3, 4, 5, 6 or more zinc fingers, each zinc finger having a recognition helix that specifically contacts a target subsite in the target gene. In certain embodiments, the zinc finger proteins comprise 4 or 5 or 6 fingers (designated F1, F2, F3, F4, F5 and F6 and ordered F1 to F4 or F5 or F6 from N-terminus to C-terminus), for example as shown in Table 1. The ZFPs as described herein may also include one or more mutations to phosphate contact residues of the zinc finger protein, for example, the nR-5Qabc mutant described in U.S. Pat. Publication No. 2018/0087072. In other embodiments, the single guide RNAs or TAL-effector DNA-binding domains may bind to a target site as described herein (e.g., target sites of Table 1 or Table 2 or Table 6 as shown in any of SEQ ID NOs:8-21 and/or 92-103) or 12 or more base pairs within any of these target sites or between paired target sites. Exemplary sgRNA target sites are shown in Table 2 (SEQ ID NOs:92-103). sgRNAs that bind to 12 or more nucleotides of the target sites shown in Table 1 or Table 2 are also provided. TALENs may be designed to target sites as described herein (target sites of Table 1 or Table 2 or Table 6) using canonical or non-canonical RVDs as described in U.S. Pat. Nos. 8,586,526 and 9,458,205. The nucleases described herein (comprising a ZFP, a TALE or a sgRNA DNA-binding domain) are capable of making genetic modifications within a TCRA gene comprising any of SEQ ID NO:8-21 and/or 92-103, including modifications (insertions and/or deletions) within any of these sequences (SEQ ID NO:8-21 and/or 92-103) and/or modifications to TCRA gene sequences flanking the target site sequences shown in SEQ ID NO:8-21 and/or 92-103, for instance modifications within exonic sequences of a TCR gene within one or more of the following sequences: AACAGT, AGTGCT, CTCCT, TTGAAA, TGGACTT and AATCCTC.

In another aspect, provided herein are B2M gene DNA-binding domains (e.g., ZFPs, TALEs and sgRNAs) that bind to a target site in a B2M gene. In certain embodiments, the DNA binding domain comprises a ZFP with the recognition helix regions in the order as shown in a single row of Table 5 or Table 8 (columns labeled “designs”, including the ZFPs of the ZFNs designated 72732; 72748; 68957; or 72678); a TAL-effector domain DNA-binding protein with the RVDs that bind to a target site as shown in the first column of Table 5 or Table 8; and/or a sgRNA that binds to a B2M target site as described herein (Table 5 or Table 8). These DNA-binding proteins can be associated with transcriptional regulatory domains to form engineered transcription factors that modulate B2M expression. Alternatively, these DNA-binding proteins can be associated with one or more nuclease (cleavage) domains to form engineered zinc finger nucleases (ZFNs), TALENs and/or CRISPR/Cas systems that bind to and cleave a B2M gene. In certain embodiments, the ZFNs, TALENs or single guide RNAs (sgRNA) of a CRISPR/Cas system bind to target sites in a human B2M gene. The DNA-binding domain of the transcription factor or nuclease (e.g., ZFP, TALE, sgRNA) may bind to a target site in a B2M gene comprising 9, 10, 11, 12 or more (e.g., 13, 14, 15, 16, 17, 18, 19, 20 or more) nucleotides of any of the target sites shown herein (e.g., Table 5 or Table 8 as shown in SEQ ID NOs: 117, 123, 126 or 127). The zinc finger proteins may include 1, 2, 3, 4, 5, 6 or more zinc fingers, each zinc finger having a recognition helix that specifically contacts a target subsite in the target gene. In certain embodiments, the zinc finger proteins comprise 4 or 5 or 6 fingers (designated F1, F2, F3, F4, F5 and F6 and ordered F1 to F4 or F5 or F6 from N-terminus to C-terminus), for example as shown in Table 5 or Table 8. The ZFPs as described herein may also include one or more mutations to phosphate contact residues of the zinc finger protein, for example, the nR-5Qabc mutant described in U.S. Pat. Publication No. 2018/0087072, including the ZFP designs (recognition helix regions and backbone mutants) of Table 8. In other embodiments, the single guide RNAs or TAL-effector DNA-binding domains may bind to a target site as described herein (e.g., target sites of Tables 5 or 8) or 12 or more base pairs within any of these target sites or between paired target sites. TALE domains may be designed to target sites as described herein (target sites of Tables 5 or 8) using canonical or non-canonical RVDs as described in U.S. Pat. Nos. 8,586,526 and 9,458,205. The nucleases described herein (comprising a ZFP, a TALE or a sgRNA DNA-binding domain) are capable of making genetic modifications within a B2M gene comprising any of the B2M target sites disclosed herein, including modifications (insertions and/or deletions) within any of these sequences and/or modifications to B2M gene sequences flanking the target site sequences shown in Tables 5 and 8 (SEQ ID NO: 117, 123, 126 or 127).

Any of the nucleases described herein may comprise a DNA-binding domain (e.g., ZFP designs of Table 6 or 8, TALE or sgRNA) as described herein and a cleavage domain and/or a cleavage half-domain (e.g., a wild-type or engineered FokI cleavage half-domain). Thus, in any of the nucleases (e.g., ZFNs, TALENs, CRISPR/Cas systems) described herein, the nuclease domain may comprise a wild-type nuclease domain or nuclease half-domain (e.g., a FokI cleavage half domain). In other embodiments, the nucleases (e.g., ZFNs, TALENs, CRISPR/Cas nucleases) comprise engineered nuclease domains or half-domains, for example engineered FokI cleavage half domains that form obligate heterodimers. See, e.g., U.S. Pat. No. 7,914,796 and 8,034,598. In certain embodiments, one or more FokI endonuclease domains of the nucleases described herein may also comprise phosphate contact mutants (e.g., R416S and/or K525S) as described in U.S. Pat. Publication No. 2018/0087072. Thus, the FokI domain of the nucleases described herein (e.g., ZFNs comprising: (i) ZFP designs as shown in Table 8, including ZFPs of the ZFNs designated 72732; 72748; 68957; or 72678 and (ii) a FokI domain) may include any combination of mutations to the FokI domain (positions numbered relative to full length FokI), including the wildtype FokI catalytic domain sequence, and also, but not limited to, the FokI domains indicated in Table 8, FokI-Sharkey (S418P+K441E); FokI ELD (Q->E at position 486, I->L at 499, N->D at position 496); FokI ELD, Sharkey (Q->E at position 486, I->L at position 499, N->D at position 496, S418P+K441E); FokI ELD, R416E (Q->E at position 486, I->L at position 499, N->D at position 496, R416E); FokI ELD, Sharkey, R416E (Q->E at position 486, I->L at position 499, N->D at position 496, S418P+K441E, R416E); FokI ELD, R416Y (Q->E at position 486, I->L at position 499, N->D at position 496, R416Y); FokI ELD, Sharkey, R416E (Q->E at position 486, I->L at position 499, N->D at position 496, S418P+K441E, R416E); FokI ELD, S418E (Q->E at position 486, I->L at position 499, N->D at position 496, S418E); FokI ELD, Sharkey partial, S418E (Q->E at position 486, I->L at position 499, N->D at position 496, K441E, S418E); FokI ELD, K525S (Q->E at position 486, I->L at position 499, N->D at position 496, K525S); FokI ELD, Sharkey K525S (Q->E at position 486, I->L at position 499, N->D at position 496, S418P+K441E, K525S); FokI ELD, I479T (Q->E at position 486, I->L at position 499, N->D at position 496, I479T); FokI ELD, Sharkey, I479T (Q->E at position 486, I->L at position 499, N->D at position 496, S418P+K441E, I479T); FokI ELD, P478D (Q->E at position 486, I->L at position 499, N->D at position 496, P478D); FokI ELD, Sharkey, P478D (Q->E at position 486, I->L at position 499, N->D at position 496, S418P+K441E, P478D); FokI ELD, Q481D (Q->E at position 486, I->L at position 499, N->D at position 496, Q481D); FokI ELD, Sharkey, Q481D (Q->E at position 486, I->L at position 499, N->D at position 496, S418P+K441E, Q481D); FokI KKR (E->K at position 490, I->K at position 538, H->R at position 537); FokI KKR Sharkey, (E->K at position 490, I->K at position 538, H->R at position 537, S418P+K441E); FokI KKR, Q481E (E->K at position 490, I->K at position 538, H->R at position 537, Q481E); FokI KKR, Sharkey Q481E (E->K at position 490, I->K at position 538, H->R at position 537, S418P+K441E, Q481E); FokI KKR, R416E (E->K at position 490, I->K at position 538, H->R at position 537, R416E); FokI KKR, Sharkey, R416E (E->K at position 490, I->K at position 538, H->R at position 537, S418P+K441E, R416E); FokI KKR, K525S (E->K at position 490, I->K at position 538, H->R at position 537, K525S); FokI KKR, Sharkey, K525S (E->K at position 490, I->K at position 538, H->R at position 537, S418P+K441E, K525S); FokI KKR, R416Y (E->K at position 490, I->K position 538, H->R at position 537, R416Y); FokI KKR, Sharkey, R416Y (E->K at position 490, I->K at position 538, H->R at position 537, S418P+K441E, R416Y); FokI, KKR I479T (E->K at position 490, I->K at position 538, H->R at position 537, I479T); FokI, KKR Sharkey I479T (E->K at position 490, I->K at position 538, H->R at position 537, S418P+K441E, I479T; FokI, KKR P478D(E->K at position 490, I->K at positions 538, H->R at position 537, P478D), FokI KKR Sharkey P478D(E->K at position 490, I->K at position 538, H->R at position 537, P478D); FokI DAD (R->D at position 487, N->D at position 496, I->A at position 499); FokI DAD Sharkey (R->D at position 487, N->D at position 496, I->A at position 499, S418P+K441E); FokI RVR (D->R at position 483, H->R at position 537, I->V at position 538); FokI RVR Sharkey (D->R at position 483, H->R at position 537, I->V at position 538, S418P+K441E). The ZFNs described herein may also include any linker sequence, including but not limited to sequences disclosed in U.S. Pat. No. 7,888,121; 7,914,796; 8,034,598; 8,623,618; 9,567,609; and U.S. Publication No. 2017/0218349, which may be used between the N- or C-terminal of the DNA-binding domain (e.g., ZFP) and N- or C-terminal of the FokI cleavage domain.

In another aspect, the disclosure provides a polynucleotide encoding any of the proteins, fusion molecules and/or components thereof (e.g., sgRNA or other DNA-binding domain) described herein. The polynucleotide may be part of a viral vector, a non-viral vector (e.g., plasmid) or be in mRNA form. Any of the polynucleotides described herein may also comprise sequences (donor, homology arms or patch sequences) for targeted insertion into the TCR α and/or the TCR β gene. In yet another aspect, a gene delivery vector comprising any of the polynucleotides described herein is provided. In certain embodiments, the vector is an adenoviral vector (e.g., an Ad5/F35 vector) or a lentiviral vector (LV) including integration competent or integration-defective lentiviral vectors or an adeno-associated vector (AAV). Thus, also provided herein are viral vectors comprising a sequence encoding a nuclease (e.g., ZFN or TALEN) and/or a nuclease system (CRISPR/Cas or Ttago) and/or a donor sequence for targeted integration into a target gene. In some embodiments, the donor sequence and the sequences encoding the nuclease are on different vectors. In other embodiments, the nucleases are supplied as polypeptides. In preferred embodiments, the polynucleotides are mRNAs. In some aspects, the mRNA may be chemically modified (See e.g., Kormann, et al. (2011) Nature Biotechnology 29(2):154-157). In other aspects, the mRNA may comprise an ARCA cap (see U.S. Pat. Nos. 7,074,596 and 8,153,773). In some aspects, the mRNA may comprise a cap introduced by enzymatic modification. The enzymatically introduced cap may comprise Cap0, Cap1 or Cap2 (see e.g., Smietanski, et al. (2014) Nature Communications 5:3004). In further aspects, the mRNA may be capped by chemical modification. In further embodiments, the mRNA may comprise a mixture of unmodified and modified nucleotides (see U.S. Patent Publication No. 2012/0195936). In still further embodiments, the mRNA may comprise a WPRE element (see U.S. Patent Publication No. 2016/0326548). In some embodiments, the mRNA is double stranded (See, e.g., Kariko, et al. (2011) Nucl Acid Res 39:e142).

In yet another aspect, the disclosure provides an isolated cell comprising any of the proteins, polynucleotides and/or vectors described herein. In certain embodiments, the cell is selected from the group consisting of a stem/progenitor cell, or a T-cell (e.g., effective or regulatory T-cell). In a still further aspect, the disclosure provides a cell or cell line which is descended from a cell or line comprising any of the nucleases, transcription factors, polynucleotides and/or vectors described herein, namely a cell or cell line descended (e.g., in culture) from a cell in which TCR and/or B2M has been inactivated by one or more ZFNs and/or in which a donor polynucleotide (e.g., ACTR and/or CAR) has been stably integrated into the genome of the cell. Thus, descendants of cells as described herein may not themselves comprise the molecule, polynucleotides and/or vectors described herein, but, in these cells, a TCR and/or B2M gene is inactivated and/or a donor polynucleotide is integrated into the genome and/or expressed.

In another aspect, described herein are methods of inactivating a TCR and/or B2M gene in a cell by introducing one or more proteins, polynucleotides and/or vectors into the cell as described herein. In certain embodiments, one or more polynucleotides encoding a ZFN (e.g., ZFN pair) as shown in Table 6 is used to modify the TCR gene in the cell and cells descended from these cells (including differentiated cells) comprise the modification(s). In other embodiments, one or more polynucleotide encoding a ZFN (e.g., ZFN pair) as shown in Table 8 is used to modify the B2M gene in the cell and cells descended from these (including differentiated cells) comprise the modification. In any of the methods described herein the nucleases may induce targeted mutagenesis, deletions of cellular DNA sequences, and/or facilitate targeted recombination at a predetermined chromosomal locus. Thus, in certain embodiments, the nucleases delete and/or insert one or more nucleotides from or into the target gene. In some embodiments a TCR and/or B2M gene is inactivated by nuclease cleavage followed by non-homologous end joining. In other embodiments, a genomic sequence in the target gene (e.g., TCR or B2M) is replaced, for example using a nuclease (or vector encoding said nuclease) as described herein and a “donor” sequence that is inserted into the gene following targeted cleavage with the nuclease. The donor sequence may be present in the nuclease vector, present in a separate vector (e.g., plasmid, linear single or double-stranded DNA, AAV, Ad or LV vector) or, alternatively, may be introduced into the cell using a different nucleic acid delivery mechanism. In some embodiments, the methods further comprise inactivating one or more additional genes (e.g., B2M) and/or integrating one or more transgenes into the genome of the cell, including, but not limited to, integration of one or more transgenes into the inactivated TCR and/or B2M gene and/or into one or more safe harbor genes. In certain embodiments, the methods described herein result in a population of cells in which at least 80-100% (or any value therebetween), including least 90-100% (or any value therebetween) of the cells include the knockout(s) and/or the integrated transgene(s).

Furthermore, any of the methods described herein can be practiced in vitro, in vivo and/or ex vivo. In certain embodiments, the methods are practiced ex vivo, for example to modify T-cells (effector or regulatory), to make them useful as therapeutics in an allogenic setting to treat a subject (e.g., a subject with cancer or autoimmune disease). Non-limiting examples of cancers that can be treated and/or prevented include lung carcinomas, pancreatic cancers, liver cancers, bone cancers, breast cancers, colorectal cancers, leukemias, ovarian cancers, lymphomas, brain cancers and the like. Non-limiting examples of autoimmune disease include transplant rejection, type 1 diabetes, irritable bowel disease/disorder, multiple sclerosis, lupus, scleroderma, rheumatoid arthritis and the like. The cells may also be used to induce immune tolerance.

In another aspect, described herein is a method of integrating one or more transgenes into a genome of an isolated cell, the method comprising: introducing, into the cell, (a) one or more donor vectors (e.g., plasmid, linear single or double-stranded DNA, AAVs, plasmids, Ads, mRNAs, etc.) comprising the one or more transgenes and (b) at least one non-naturally occurring nuclease in mRNA form, wherein the at least one nuclease cleaves the genome of the cell such that the one or more transgenes are integrated into the genome of the cell (e.g., into a TCR receptor), wherein the donor vector is introduced into introduced into the electroporation buffer comprising the isolated cell and the mRNA immediately before or immediately after electroporation of the nuclease into the cell. In certain embodiments, the donor vector is introduced into the electroporation buffer after electroporation and prior to transfer of the cells into a culture medium. See, e.g., U.S. Pat. Publication Nos. 2015/0174169 and 2015/0110762. The methods may be used to introduce the transgene(s) into any genomic location, including, but not limited to, a TCR gene, a B2M gene and/or a safe harbor gene (e.g., AAVS1, Rosa, albumin, CCR5, CXCR4, etc.).

### BRIEF DESCRIPTION OF THE DRAWINGS

FIGS. 1A and 1B are a depiction of the TCRA gene showing the locations of the sites targeted by the nucleases. FIG. 1A is an illustration of the processing of the TCRA gene from the germline form to that of a mature T cell and indicates the general target of the nucleases. FIG. 1B (SEQ ID NOs:116 (exon c1), 187 (exon c2) and 118 (exon c3)) shows the regions between the target sites in the constant region sequence. The sequence shown in uppercase black lettering is the sequence of the indicated exon sequence, while the sequence in lowercase grey lettering is the adjoining intron sequence.

FIGS. 2A and 2B are graphs depicting the percent of each site modified in T cells treated with ZFNs specific for TCRA sites A, B and D (FIG. 2A) and sites E, F and G (FIG. 2B). Many of the pairs gave modification rates of 80% or greater.

FIG. 3 depicts the percent of CD3 negative T cells following treatment with the TCRA-specific ZFN pairs as analyzed by FACS analysis.

FIG. 4 is a graph showing the high degree of correlation in T cells between levels of TCRA sequence modification as measured via high throughput sequencing and loss of CD3 expression as measured by fluorescence activated cell sorting.

FIGS. 5A through 5D are graphs depicting the growth of T cells following treatment with the TCRA-specific ZFN grouped according to the target site in the TCRA gene.

FIG. 6 shows results from TRAC (TCRA) and B2M double knockout and targeted integration of a donor into either the TRAC (TCRA) or B2M locus.

FIG. 7 shows FACS results from TRAC (TCRA) and B2M double knockout and targeted integration of a donor into either the TRAC (TCRA) or B2M locus. FACS results are shown for the indicated conditions (from left to right of upper panels: control (sham); TRAC and B2M ZFNs without a donor; TRAC and B2M ZFNs with donor targeted to B2M; and TRAC and B2M ZFNs with donor targeted to TRAC). The lower left quadrant of the top row of FACs plots shows cells with a double (TRAC/B2M) knockout and the right half of the bottom row of FACs plots shows cells with a double knockout and targeted integration. The percentage of cells is also indicated by arrows pointing towards the appropriate section of the FACs plot. As indicated by the arrows, 85-90% or more of cells were double KO and were also positive for targeted integration.

### DETAILED DESCRIPTION

Disclosed herein are compositions and methods for generating cells in which expression of a TCR gene is modulated such that the cells no longer comprise a TCR on their cell surfaces and/or in which expression of a B2M gene is modulated such that the cells no longer express B2M. Cells modified in this manner can be used as therapeutics, for example, transplants, as the lack of a TCR complex prevents or reduces an HLA-based immune response. Additionally, other genes of interest (e.g., transgenes) may be inserted into cells in which the TCR and/or B2M gene have been manipulated. One or more additional (non-TCR and/or B2M) genes (e.g., other TCR, B2M, PD1, CTLA4, HLA genes, safe harbor genes, etc.) may be modified via knock out and/or targeted insertion of exogenous sequences. Exogenous sequences can include chimeric antigen receptors for integration into the modified cells, which can be used to treat cancer and autoimmune disorders.

### General

Practice of the methods, as well as preparation and use of the compositions disclosed herein employ, unless otherwise indicated, conventional techniques in molecular biology, biochemistry, chromatin structure and analysis, computational chemistry, cell culture, recombinant DNA and related fields as are within the skill of the art. These techniques are fully explained in the literature. See, for example, Sambrook et al., MOLECULAR CLONING: A LABORATORY MANUAL, Second edition, Cold Spring Harbor Laboratory Press, 1989 and Third edition, 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolffe, CHROMATIN STRUCTURE AND FUNCTION, Third edition, Academic Press, San Diego, 1998; METHODS IN ENZYMOLOGY, Vol. 304, “Chromatin” (P.M. Wassarman and A. P. Wolffe, eds.), Academic Press, San Diego, 1999; and METHODS IN MOLECULAR BIOLOGY, Vol. 119, “Chromatin Protocols” (P.B. Becker, ed.) Humana Press, Totowa, 1999.

### Definitions

The terms “nucleic acid,” “polynucleotide,” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

The terms “polypeptide,” “peptide” and “protein” are used interchangeably to refer to a polymer of amino acid residues. The term also applies to amino acid polymers in which one or more amino acids are chemical analogues or modified derivatives of corresponding naturally-occurring amino acids.

“Binding” refers to a sequence-specific, non-covalent interaction between macromolecules (e.g., between a protein and a nucleic acid). Not all components of a binding interaction need be sequence-specific (e.g., contacts with phosphate residues in a DNA backbone), as long as the interaction as a whole is sequence-specific. Such interactions are generally characterized by a dissociation constant (K_{d}) of 10⁻⁶ M⁻¹ or lower. “Affinity” refers to the strength of binding: increased binding affinity being correlated with a lower K_{d}. “Non-specific binding” refers to, non-covalent interactions that occur between any molecule of interest (e.g., an engineered nuclease) and a macromolecule (e.g., DNA) that are not dependent on target sequence.

A “DNA binding molecule” is a molecule that can bind to DNA. Such DNA binding molecule can be a polypeptide, a domain of a protein, a domain within a larger protein or a polynucleotide. In some embodiments, the polynucleotide is DNA, while in other embodiments, the polynucleotide is RNA. In some embodiments, the DNA binding molecule is a protein domain of a nuclease (e.g., the FokI domain), while in other embodiments, the DNA binding molecule is a guide RNA component of an RNA-guided nuclease (e.g., Cas9 or Cfp1).

A “binding protein” is a protein that is able to bind non-covalently to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding and protein-binding activity.

A “zinc finger DNA binding protein” (or binding domain) is a protein, or a domain within a larger protein, that binds DNA in a sequence-specific manner through one or more zinc fingers, which are regions of amino acid sequence within the binding domain whose structure is stabilized through coordination of a zinc ion. Thus, each zinc finger of a multi-finger ZFP includes a recognition helix region for binding to DNA within a backbone. The term zinc finger DNA binding protein is often abbreviated as zinc finger protein or ZFP. The term “zinc finger nuclease” includes one ZFN as well as a pair of ZFNs (the members of the pair are referred to as “left and right” or “first and second” or “pair”) that dimerize to cleave the target gene.

A “TALE DNA binding domain” or “TALE” is a polypeptide comprising one or more TALE repeat domains/units. The repeat domains, each comprising a repeat variable diresidue (RVD), are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein. TALE proteins may be designed to bind to a target site using canonical or non-canonical RVDs within the repeat units. See, e.g., U.S. Pat. Nos. 8,586,526 and 9,458,205. Zinc finger and TALE DNA-binding domains can be “engineered” to bind to a predetermined nucleotide sequence, for example via engineering (altering one or more amino acids) of the recognition helix region of a naturally occurring zinc finger protein or by engineering of the amino acids involved in DNA binding (the repeat variable diresidue or RVD region). Therefore, engineered zinc finger proteins or TALE proteins are proteins that are non-naturally occurring. Non-limiting examples of methods for engineering zinc finger proteins and TALEs are design and selection. A designed protein is a protein not occurring in nature whose design/composition results principally from rational criteria. Rational criteria for design include application of substitution rules and computerized algorithms for processing information in a database storing information of existing ZFP or TALE designs (canonical and non-canonical RVDs) and binding data. See, for example, U.S. Pat. Nos. 9,458,205; 8,586,526; 6,140,081; 6,453,242; and 6,534,261; see also International Patent Publication Nos. WO 98/53058; WO 98/53059; WO 98/53060; WO 02/16536; and WO 03/016496. The term “TALEN” includes one TALEN as well as a pair of TALENs (the members of the pair are referred to as “left and right” or “first and second” or “pair”) that dimerize to cleave the target gene.

A “selected” zinc finger protein, TALE protein or CRISPR/Cas system is not found in nature and whose production results primarily from an empirical process such as phage display, interaction trap or hybrid selection. See e.g., U.S. Pat. Nos. 5,789,538; 5,925,523; 6,007,988; 6,013,453; 6,200,759 and International Patent Publication Nos. WO 95/19431; WO 96/06166; WO 98/53057; WO 98/54311; WO 00/27878; WO 01/60970; WO 01/88197; and WO 02/099084.

“TtAgo” is a prokaryotic Argonaute protein thought to be involved in gene silencing. TtAgo is derived from the bacteria Thermus thermophilus. See, e.g., Swarts, et al., ibid, G. Sheng, et al. (2013) Proc. Natl. Acad. Sci. U.S.A. 111, 652). A “TtAgo system” is all the components required including e.g., guide DNAs for cleavage by a TtAgo enzyme.

“Recombination” refers to a process of exchange of genetic information between two polynucleotides. For the purposes of this disclosure, “homologous recombination (HR)” refers to the specialized form of such exchange that takes place, for example, during repair of double-strand breaks in cells via homology-directed repair mechanisms. This process requires nucleotide sequence homology, uses a “donor” molecule to template repair of a “target” molecule (i.e., the one that experienced the double-strand break), and is variously known as “non-crossover gene conversion” or “short tract gene conversion,” because it leads to the transfer of genetic information from the donor to the target. Without wishing to be bound by any particular theory, such transfer can involve mismatch correction of heteroduplex DNA that forms between the broken target and the donor, and/or “synthesis-dependent strand annealing,” in which the donor is used to resynthesize genetic information that will become part of the target, and/or related processes. Such specialized HR often results in an alteration of the sequence of the target molecule such that part or all of the sequence of the donor polynucleotide is incorporated into the target polynucleotide.

In the methods of the disclosure, one or more targeted nucleases as described herein create a double-stranded break (DSB) in the target sequence (e.g., cellular chromatin) at a predetermined site (e.g., a gene or locus of interest), and a “donor” polynucleotide, having homology to the nucleotide sequence in the region of the break, can be introduced into the cell. The presence of the DSB has been shown to facilitate integration of the donor sequence. Optionally, the construct has homology to the nucleotide sequence in the region of the break. The donor sequence may be physically integrated or, alternatively, the donor polynucleotide is used as a template for repair of the break via homologous recombination, resulting in the introduction of all or part of the nucleotide sequence as in the donor into the cellular chromatin. Thus, a first sequence in cellular chromatin can be altered and, in certain embodiments, can be converted into a sequence present in a donor polynucleotide. Thus, the use of the terms “replace” or “replacement” can be understood to represent replacement of one nucleotide sequence by another, (i.e., replacement of a sequence in the informational sense), and does not necessarily require physical or chemical replacement of one polynucleotide by another.

In any of the methods described herein, additional pairs of zinc-finger proteins can be used for additional double-stranded cleavage of additional target sites within the cell.

In certain embodiments of methods for targeted recombination and/or replacement and/or alteration of a sequence in a region of interest in cellular chromatin, a chromosomal sequence is altered by homologous recombination with an exogenous “donor” nucleotide sequence. Such homologous recombination is stimulated by the presence of a double-stranded break in cellular chromatin, if sequences homologous to the region of the break are present.

In any of the methods described herein, the first nucleotide sequence (the “donor sequence”) can contain sequences that are homologous, but not identical, to genomic sequences in the region of interest, thereby stimulating homologous recombination to insert a non-identical sequence in the region of interest. Thus, in certain embodiments, portions of the donor sequence that are homologous to sequences in the region of interest exhibit between about 80 to 99% (or any integer therebetween) sequence identity to the genomic sequence that is replaced. In other embodiments, the homology between the donor and genomic sequence is higher than 99%, for example if only 1 nucleotide differs as between donor and genomic sequences of over 100 contiguous base pairs. In certain cases, a non-homologous portion of the donor sequence can contain sequences not present in the region of interest, such that new sequences are introduced into the region of interest. In these instances, the non-homologous sequence is generally flanked by sequences of 50-1,000 base pairs (or any integral value therebetween) or any number of base pairs greater than 1,000, that are homologous or identical to sequences in the region of interest. In other embodiments, the donor sequence is non-homologous to the first sequence and is inserted into the genome by non-homologous recombination mechanisms.

Any of the methods described herein can be used for partial or complete inactivation of one or more target sequences in a cell by targeted integration of donor sequence that disrupts expression of the gene(s) of interest. Cell lines with partially or completely inactivated genes are also provided.

Furthermore, the methods of targeted integration as described herein can also be used to integrate one or more exogenous sequences. The exogenous nucleic acid sequence can comprise, for example, one or more genes or cDNA molecules, or any type of coding or noncoding sequence, as well as one or more control elements (e.g., promoters). In addition, the exogenous nucleic acid sequence may produce one or more RNA molecules (e.g., small hairpin RNAs (shRNAs), inhibitory RNAs (RNAis), microRNAs (miRNAs), etc.).

“Cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, fusion polypeptides are used for targeted double-stranded DNA cleavage.

A “cleavage half-domain” is a polypeptide sequence which, in conjunction with a second polypeptide (either identical or different) forms a complex having cleavage activity (preferably double-strand cleavage activity). The terms “first and second cleavage half-domains;” “+ and - cleavage half-domains” and “right and left cleavage half-domains” are used interchangeably to refer to pairs of cleavage half-domains that dimerize.

An “engineered cleavage half-domain” is a cleavage half-domain that has been modified so as to form obligate heterodimers with another cleavage half-domain (e.g., another engineered cleavage half-domain). See, also, U.S. Pat. Nos. 7,888,121; 7,914,796; 8,034,598; 8,623,618 and U.S. Pat. Publication No. 2011/0201055, incorporated herein by reference in their entireties.

The term “sequence” refers to a nucleotide sequence of any length, which can be DNA or RNA; can be linear, circular or branched and can be either single-stranded or double stranded. The term “donor sequence” refers to a nucleotide sequence that is inserted into a genome. A donor sequence can be of any length, for example between 2 and 10,000 nucleotides in length (or any integer value therebetween or thereabove), preferably between about 100 and 1,000 nucleotides in length (or any integer therebetween), more preferably between about 200 and 500 nucleotides in length.

“Chromatin” is the nucleoprotein structure comprising the cellular genome. Cellular chromatin comprises nucleic acid, primarily DNA, and protein, including histones and non-histone chromosomal proteins. The majority of eukaryotic cellular chromatin exists in the form of nucleosomes, wherein a nucleosome core comprises approximately 150 base pairs of DNA associated with an octamer comprising two each of histones H2A, H2B, H3 and H4; and linker DNA (of variable length depending on the organism) extends between nucleosome cores. A molecule of histone H1 is generally associated with the linker DNA. For the purposes of the present disclosure, the term “chromatin” is meant to encompass all types of cellular nucleoprotein, both prokaryotic and eukaryotic. Cellular chromatin includes both chromosomal and episomal chromatin.

A “chromosome,” is a chromatin complex comprising all or a portion of the genome of a cell. The genome of a cell is often characterized by its karyotype, which is the collection of all the chromosomes that comprise the genome of the cell. The genome of a cell can comprise one or more chromosomes.

An “episome” is a replicating nucleic acid, nucleoprotein complex or other structure comprising a nucleic acid that is not part of the chromosomal karyotype of a cell. Examples of episomes include plasmids and certain viral genomes.

A “target site” or “target sequence” is a nucleic acid sequence that defines a portion of a nucleic acid to which a binding molecule will bind, provided sufficient conditions for binding exist. For example, the sequence 5′ GAATTC 3′ is a target site for the Eco RI restriction endonuclease.

An “exogenous” molecule is a molecule that is not normally present in a cell, but can be introduced into a cell by one or more genetic, biochemical or other methods. “Normal presence in the cell” is determined with respect to the particular developmental stage and environmental conditions of the cell. Thus, for example, a molecule that is present only during embryonic development of muscle is an exogenous molecule with respect to an adult muscle cell. Similarly, a molecule induced by heat shock is an exogenous molecule with respect to a non-heat-shocked cell. An exogenous molecule can comprise, for example, a functioning version of a malfunctioning endogenous molecule or a malfunctioning version of a normally-functioning endogenous molecule.

An exogenous molecule can be, among other things, a small molecule, such as is generated by a combinatorial chemistry process, or a macromolecule such as a protein, nucleic acid, carbohydrate, lipid, glycoprotein, lipoprotein, polysaccharide, any modified derivative of the above molecules, or any complex comprising one or more of the above molecules. Nucleic acids include DNA and RNA, can be single- or double-stranded; can be linear, branched or circular; and can be of any length. See, e.g., U.S. Pat. Nos. 8,703,489 and 9,255,259. Nucleic acids include those capable of forming duplexes, as well as triplex-forming nucleic acids. See, for example, U.S. Pat. Nos. 5,176,996 and 5,422,251. Proteins include, but are not limited to, DNA-binding proteins, transcription factors, chromatin remodeling factors, methylated DNA binding proteins, polymerases, methylases, demethylases, acetylases, deacetylases, kinases, phosphatases, integrases, recombinases, ligases, topoisomerases, gyrases and helicases.

An exogenous molecule can be the same type of molecule as an endogenous molecule, e.g., an exogenous protein or nucleic acid. For example, an exogenous nucleic acid can comprise an infecting viral genome, a plasmid or episome introduced into a cell, or a chromosome that is not normally present in the cell. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer. An exogenous molecule can also be the same type of molecule as an endogenous molecule but derived from a different species than the cell is derived from. For example, a human nucleic acid sequence may be introduced into a cell line originally derived from a mouse or hamster.

By contrast, an “endogenous” molecule is one that is normally present in a particular cell at a particular developmental stage under particular environmental conditions. For example, an endogenous nucleic acid can comprise a chromosome, the genome of a mitochondrion, chloroplast or other organelle, or a naturally-occurring episomal nucleic acid. Additional endogenous molecules can include proteins, for example, transcription factors and enzymes.

A “fusion” molecule is a molecule in which two or more subunit molecules are linked, preferably covalently. The subunit molecules can be the same chemical type of molecule, or can be different chemical types of molecules. Examples of the first type of fusion molecule include, but are not limited to, fusion proteins (for example, a fusion between a ZFP or TALE DNA-binding domain and one or more activation domains) and fusion nucleic acids (for example, a nucleic acid encoding the fusion protein described supra). Examples of the second type of fusion molecule include, but are not limited to, a fusion between a triplex-forming nucleic acid and a polypeptide, and a fusion between a minor groove binder and a nucleic acid. The term also includes systems in which a polynucleotide component associates with a polypeptide component to form a functional molecule (e.g., a CRISPR/Cas system in which a single guide RNA associates with a functional domain to modulate gene expression).

Expression of a fusion protein in a cell can result from delivery of the fusion protein to the cell or by delivery of a polynucleotide encoding the fusion protein to a cell, wherein the polynucleotide is transcribed, and the transcript is translated, to generate the fusion protein. Trans-splicing, polypeptide cleavage and polypeptide ligation can also be involved in expression of a protein in a cell. Methods for polynucleotide and polypeptide delivery to cells are presented elsewhere in this disclosure.

A “gene,” for the purposes of the present disclosure, includes a DNA region encoding a gene product (see infra), as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

A “safe harbor” locus is a locus within the genome wherein a gene may be inserted without any deleterious effects on the host cell. Most beneficial is a safe harbor locus in which expression of the inserted gene sequence is not perturbed by any read-through expression from neighboring genes. Non-limiting examples of safe harbor loci that are targeted by nuclease(s) include CCR5, CCR5, HPRT, AAVS1, Rosa and albumin. See, e.g., U.S. Pat. Nos. 8,771,985; 8,110,379; 7,951,925; U.S. Pat. Publication Nos. 2010/0218264; 2011/0265198; 2013/0137104; 2013/0122591; 2013/0177983; 2013/0177960; 2015/0056705; and 2015/0159172).

“Gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation. “Modulation” or “modification” of gene expression refers to a change in the activity of a gene. Modulation of expression can include, but is not limited to, gene activation and gene repression, including by modification of the gene via binding of an exogenous molecule (e.g., engineered transcription factor). Modulation may also be achieved by modification of the gene sequence via genome editing (e.g., cleavage, alteration, inactivation, random mutation). Gene inactivation refers to any reduction in gene expression as compared to a cell that has not been modified as described herein. Thus, gene inactivation may be partial or complete.

A “region of interest” is any region of cellular chromatin, such as, for example, a gene or a non-coding sequence within or adjacent to a gene, in which it is desirable to bind an exogenous molecule. Binding can be for the purposes of targeted DNA cleavage and/or targeted recombination. A region of interest can be present in a chromosome, an episome, an organellar genome (e.g., mitochondrial, chloroplast), or an infecting viral genome, for example. A region of interest can be within the coding region of a gene, within transcribed non-coding regions such as, for example, leader sequences, trailer sequences or introns, or within non-transcribed regions, either upstream or downstream of the coding region. A region of interest can be as small as a single nucleotide pair or up to 2,000 nucleotide pairs in length, or any integral value of nucleotide pairs.

“Eukaryotic” cells include, but are not limited to, fungal cells (such as yeast), plant cells, animal cells, mammalian cells and human cells (e.g., T-cells).

The terms “operative linkage” and “operatively linked” (or “operably linked”) are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence, even though they are not contiguous.

With respect to fusion polypeptides, the term “operatively linked” can refer to the fact that each of the components performs the same function in linkage to the other component as it would if it were not so linked. For example, with respect to a fusion polypeptide in which a DNA-binding domain (e.g., ZFP, TALE) is fused to an activation domain, the DNA-binding domain and the activation domain are in operative linkage if, in the fusion polypeptide, the DNA-binding domain portion is able to bind its target site and/or its binding site, while the activation domain is able to up-regulate gene expression. When a fusion polypeptide in which a DNA-binding domain is fused to a cleavage domain, the DNA-binding domain and the cleavage domain are in operative linkage if, in the fusion polypeptide, the DNA-binding domain portion is able to bind its target site and/or its binding site, while the cleavage domain is able to cleave DNA in the vicinity of the target site. Similarly, with respect to a fusion polypeptide in which a DNA-binding domain is fused to an activation or repression domain, the DNA-binding domain and the activation or repression domain are in operative linkage if, in the fusion polypeptide, the DNA-binding domain portion is able to bind its target site and/or its binding site, while the activation domain is able to upregulate gene expression or the repression domain is able to downregulate gene expression.

A “functional fragment” of a protein, polypeptide or nucleic acid is a protein, polypeptide or nucleic acid whose sequence is not identical to the full-length protein, polypeptide or nucleic acid, yet retains the same function as the full-length protein, polypeptide or nucleic acid. A functional fragment can possess more, fewer, or the same number of residues as the corresponding native molecule, and/or can contain one or more amino acid or nucleotide substitutions. Methods for determining the function of a nucleic acid (e.g., coding function, ability to hybridize to another nucleic acid) are well-known in the art. Similarly, methods for determining protein function are well-known. For example, the DNA-binding function of a polypeptide can be determined, for example, by filter-binding, electrophoretic mobility-shift, or immunoprecipitation assays. DNA cleavage can be assayed by gel electrophoresis. See Ausubel, et al., supra. The ability of a protein to interact with another protein can be determined, for example, by co-immunoprecipitation, two-hybrid assays or complementation, both genetic and biochemical. See, for example, Fields, et al. (1989) Nature 340:245-246; U.S. Pat. No. 5,585,245 and International Patent Publication No. WO 98/44350.

A “vector” is capable of transferring gene sequences to target cells. Typically, “vector construct,” “expression vector,” and “gene transfer vector,” mean any nucleic acid construct capable of directing the expression of a gene of interest and which can transfer gene sequences to target cells. Thus, the term includes cloning, and expression vehicles, as well as integrating vectors.

A “reporter gene” or “reporter sequence” refers to any sequence that produces a protein product that is easily measured, preferably although not necessarily in a routine assay. Suitable reporter genes include, but are not limited to, sequences encoding proteins that mediate antibiotic resistance (e.g., ampicillin resistance, neomycin resistance, G418 resistance, puromycin resistance), sequences encoding colored or fluorescent or luminescent proteins (e.g., green fluorescent protein, enhanced green fluorescent protein, red fluorescent protein, luciferase), and proteins which mediate enhanced cell growth and/or gene amplification (e.g., dihydrofolate reductase). Epitope tags include, for example, one or more copies of FLAG, His, myc, Tap, HA or any detectable amino acid sequence. “Expression tags” include sequences that encode reporters that may be operably linked to a desired gene sequence in order to monitor expression of the gene of interest.

The terms “subject” and “patient” are used interchangeably and refer to mammals such as human patients and non-human primates, as well as experimental animals such as rabbits, dogs, cats, rats, mice, and other animals. Accordingly, the term “subject” or “patient” as used herein means any mammalian patient or subject to which the expression cassettes of the invention can be administered. Subjects of the present invention include those with a disorder or those at risk for developing a disorder.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage. Cancer and graft versus host disease are non-limiting examples of conditions that may be treated using the compositions and methods described herein. Thus, “treating” and “treatment includes:
(i) preventing the disease or condition from occurring in a mammal, in particular, when such mammal is predisposed to the condition but has not yet been diagnosed as having it;
(ii) inhibiting the disease or condition, i.e., arresting its development;
(iii) relieving the disease or condition, i.e., causing regression of the disease or condition; or
(iv) relieving the symptoms resulting from the disease or condition, i.e., relieving pain without addressing the underlying disease or condition.

As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

A “pharmaceutical composition” refers to a formulation of a compound of the invention and a medium generally accepted in the art for the delivery of the biologically active compound to mammals, e.g., humans. Such a medium includes all pharmaceutically acceptable carriers, diluents or excipients therefor.

“Effective amount” or “therapeutically effective amount” refers to that amount of a compound of the invention which, when administered to a mammal, preferably a human, is sufficient to effect treatment in the mammal, preferably a human. The amount of a composition of the invention which constitutes a “therapeutically effective amount” will vary depending on the compound, the condition and its severity, the manner of administration, and the age of the mammal to be treated, but can be determined routinely by one of ordinary skill in the art having regard to his own knowledge and to this disclosure.

### DNA-Binding Domains

Described herein are compositions comprising a DNA-binding domain that specifically binds to a target site in any gene comprising a HLA gene or a HLA regulator. Any DNA-binding domain can be used in the compositions and methods disclosed herein, including but not limited to a zinc finger DNA-binding domain, a TALE DNA binding domain, the DNA-binding portion (sgRNA) of a CRISPR/Cas nuclease, or a DNA-binding domain from a meganuclease. The DNA-binding domain may bind to any target sequence within the gene, including, but not limited to, a target sequence of 12 or more nucleotides as shown in any of target sites disclosed herein (SEQ ID NO:8-21 and/or 92-103).

In certain embodiments, the DNA binding domain comprises a zinc finger protein. Preferably, the zinc finger protein is non-naturally occurring in that it is engineered to bind to a target site of choice. See, for example, Beerli, et al. (2002) Nature Biotechnol. 20:135-141; Pabo, et al. (2001) Ann. Rev. Biochem. 70:313-340; Isalan, et al. (2001) Nature Biotechnol. 19:656-660; Segal, et al. (2001) Curr. Opin. Biotechnol. 12:632-637; Choo, et al. (2000) Curr. Opin. Struct. Biol. 10:411-416; U.S. Pat. Nos. 6,453,242; 6,534,261; 6,599,692; 6,503,717; 6,689,558; 7,030,215; 6,794,136; 7,067,317; 7,262,054; 7,070,934; 7,361,635; and 7,253,273; and U.S. Pat. Publication Nos. 2005/0064474; 2007/0218528; ; and 2005/0267061, all incorporated herein by reference in their entireties. In certain embodiments, the DNA-binding domain comprises a zinc finger protein disclosed in U.S. Pat. Publication No. 2012/0060230 (e.g., Table 1), incorporated by reference in its entirety herein. In other embodiments, the DNA-binding domain comprises the ZFP component (referred to as “designs”) and including recognition helix regions and backbones as set forth in the ZFNs of Tables 1, 2, 4, 5, 6 or 8, including but not limited to the ZFP domains of ZFNs 72732; 72748; 68957; or 72678.

An engineered zinc finger binding domain can have a novel binding specificity, compared to a naturally-occurring zinc finger protein. Engineering methods include, but are not limited to, rational design and various types of selection. Rational design includes, for example, using databases comprising triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, for example, U.S. Pat. Nos. 6,453,242 and 6,534,261, incorporated by reference herein in their entireties.

Exemplary selection methods, including phage display and two-hybrid systems, are disclosed in U.S. Pat. Nos. 5,789,538; 5,925,523; 6,007,988; 6,013,453; 6,410,248; 6,140,466; 6,200,759; and 6,242,568; as well as International Patent Publication Nos. WO 98/37186; WO 98/53057; WO 00/27878; and WO 01/88197 and GB 2,338,237. In addition, enhancement of binding specificity for zinc finger binding domains has been described, for example, in U.S. Pat. No. 6,794,136.

In addition, as disclosed in these and other references, zinc finger domains and/or multi-fingered zinc finger proteins may be linked together using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The proteins described herein may include any combination of suitable linkers between the individual zinc fingers of the protein. In addition, enhancement of binding specificity for zinc finger binding domains has been described, for example, in U.S. Pat. No. 6,794,136.

Selection of target sites; ZFPs and methods for design and construction of fusion proteins (and polynucleotides encoding same) are known to those of skill in the art and described in detail in U.S. Pat. Nos. 6,140,081; 5,789,538; 6,453,242; 6,534,261; 5,925,523; 6,007,988; 6,013,453; 6,200,759; and International Patent Publication Nos. WO 95/19431; WO 96/06166; WO 98/53057; WO 98/54311; WO 00/27878; WO 01/60970; WO 01/88197; WO 02/099084; WO 98/53058; WO 98/53059; WO 98/53060; WO 02/16536; and WO 03/016496.

In addition, as disclosed in these and other references, zinc finger domains and/or multi-fingered zinc finger proteins may be linked together using any suitable linker sequences, including for example, linkers of 5 or more amino acids in length. See, also, U.S. Pat. Nos. 6,479,626; 6,903,185; 7,153,949; 7,888,121; 7,914,796; 8,034,598; 8,623,618; 9,567,609; and U.S. Pat. Publication No. 2017/0218349 for exemplary linker sequences. The proteins described herein may include any combination of suitable linkers between the individual zinc fingers of the protein.

In certain embodiments, the DNA-binding domain is an engineered zinc finger protein that binds (in a sequence-specific manner) to a target site in a TCR gene or TCR regulatory gene and modulates expression of a TCR gene. In some embodiments, the zinc finger protein binds to a target site in TCRA, while in other embodiments, the zinc finger binds to a target site in TRBC. In other embodiments, the DNA-binding domain is an engineered zinc finger protein that binds (in a sequence-specific manner) to a target site in a B2M gene and modulates expression of a B2M gene. Non-limiting exemplary embodiments of these DNA-binding domains are shown in Tables 1, 2 and 6 (TCR) and Tables 5 and 8 (B2M). In certain embodiments, the ZFP comprises the ZFP portion of the ZFNs designated 72732; 72748; 68957; or 72678.

Usually, the ZFPs include at least three fingers. Certain of the ZFPs include four, five or six fingers. The ZFPs that include three fingers typically recognize a target site that includes 9 or 10 nucleotides; ZFPs that include four fingers typically recognize a target site that includes 12 to 14 nucleotides; while ZFPs having six fingers can recognize target sites that include 18 to 21 nucleotides. The ZFPs can also be fusion proteins that include one or more regulatory domains, which domains can be transcriptional activation or repression domains.

In some embodiments, the DNA-binding domain may be derived from a nuclease. For example, the recognition sequences of homing endonucleases and meganucleases such as I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII are known. See also U.S. Pat. No. 5,420,032; U.S. Pat. No. 6,833,252; Belfort, et al. (1997) Nucleic Acids Res. 25:3379-3388; Dujon, et al. (1989) Gene 82: 115-118; Perler, et al. (1994) Nucleic Acids Res. 22, 1125-1127; Jasin (1996) Trends Genet. 12:224-228; Gimble, et al. (1996) J. Mol. Biol. 263:163-180; Argast, et al. (1998) J. Mol. Biol. 280:345-353 and the New England Biolabs catalogue. In addition, the DNA-binding specificity of homing endonucleases and meganucleases can be engineered to bind non-natural target sites. See, for example, Chevalier, et al. (2002) Molec. Cell 10:895-905; Epinat, et al. (2003) Nucleic Acids Res. 31:2952-2962; Ashworth, et al. (2006) Nature 441:656-659; Pâques, et al. (2007) Current Gene Therapy 7:49-66; U.S. Pat. Publication No. 2007/0117128.

In other embodiments, the DNA binding domain comprises an engineered domain from a TAL effector similar to those derived from the plant pathogens Xanthomonas (see Boch, et al. (2009) Science 326: 1509-1512 and Moscou and Bogdanove (2009) Science 326:1501) and Ralstonia (see Heuer, et al. (2007) Applied and Environmental Microbiology 73(13): 4379-4384); U.S. Pat. Publication Nos. 2011/0301073 and 2011/0145940. The plant pathogenic bacteria of the genus Xanthomonas are known to cause many diseases in important crop plants. Pathogenicity of Xanthomonas depends on a conserved type III secretion (T3S) system which injects more than 25 different effector proteins into the plant cell. Among these injected proteins are transcription activator-like effectors (TALE) which mimic plant transcriptional activators and manipulate the plant transcriptome (see Kay, et al. (2007) Science 318:648-651). These proteins contain a DNA binding domain and a transcriptional activation domain. One of the most well characterized TALEs is AvrBs3 from Xanthomonas campestgris pv. Vesicatoria (see Bonas, et al. (1989) Mol Gen Genet 218: 127-136 and InternationalPatent Publication No. WO 2010/079430). TALEs contain a centralized domain of tandem repeats, each repeat containing approximately 34 amino acids, which are key to the DNA binding specificity of these proteins. In addition, they contain a nuclear localization sequence and an acidic transcriptional activation domain (for a review see Schornack S., et al. (2006) J Plant Physiol 163(3): 256-272). In addition, in the phytopathogenic bacteria Ralstonia solanacearum two genes, designated brg11 and hpx17 have been found that are homologous to the AvrBs3 family of Xanthomonas in the R. solanacearum biovar 1 strain GMI1000 and in the biovar 4 strain RS1000 (See Heuer, et al. (2007) Appl andEnvir Micro 73(13):4379-4384). These genes are 98.9% identical in nucleotide sequence to each other but differ by a deletion of 1,575 bp in the repeat domain of hpx17. However, both gene products have less than 40% sequence identity with AvrBs3 family proteins of Xanthomonas.

Specificity of these TAL effectors depends on the sequences found in the tandem repeats. The repeated sequence comprises approximately 102 base pairs and the repeats are typically 91-100% homologous with each other (Bonas, et al., ibid). Polymorphism of the repeats is usually located at positions 12 and 13 and there appears to be a one-to-one correspondence between the identity of the hypervariable diresidues (the repeat variable diresidue or RVD region) at positions 12 and 13 with the identity of the contiguous nucleotides in the TAL-effector’s target sequence (see Moscou and Bogdanove (2009) Science 326:1501 and Boch, et al. (2009) Science 326:1509-1512). Experimentally, the natural code for DNA recognition of these TAL-effectors has been determined such that an HD sequence at positions 12 and 13 (Repeat Variable Diresidue or RVD) leads to a binding to cytosine (C), NG binds to T, NI to A, C, G or T, NN binds to A or G, and ING binds to T. These DNA binding repeats have been assembled into proteins with new combinations and numbers of repeats, to make artificial transcription factors that are able to interact with new sequences and activate the expression of a non-endogenous reporter gene in plant cells (Boch, et al., ibid). Engineered TAL proteins have been linked to a FokI cleavage half domain to yield a TAL effector domain nuclease fusion (TALEN), including TALENs with atypical RVDs. See, e.g., U.S. Pat. No. 8,586,526.

In some embodiments, the TALEN comprises an endonuclease (e.g., FokI) cleavage domain or cleavage half-domain. In other embodiments, the TALE-nuclease is a mega TAL. These mega TAL nucleases are fusion proteins comprising a TALE DNA binding domain and a meganuclease cleavage domain. The meganuclease cleavage domain is active as a monomer and does not require dimerization for activity. (See Boissel, et al. (2013) Nucl Acid Res: 1-13, doi: 10.1093/nar/gkt1224).

In still further embodiments, the nuclease comprises a compact TALEN. These are single chain fusion proteins linking a TALE DNA binding domain to a TevI nuclease domain. The fusion protein can act as either a nickase localized by the TALE region, or can create a double strand break, depending upon where the TALE DNA binding domain is located with respect to the TevI nuclease domain (see Beurdeley, et al. (2013) Nat Comm 4:1762 DOI: 10.1038/ncomms2782). In addition, the nuclease domain may also exhibit DNA-binding functionality. Any TALENs may be used in combination with additional TALENs (e.g., one or more TALENs (cTALENs or FokI-TALENs) with one or more mega-TALEs.

In addition, as disclosed in these and other references, zinc finger domains and/or multi-fingered zinc finger proteins or TALEs may be linked together using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The proteins described herein may include any combination of suitable linkers between the individual zinc fingers of the protein. In addition, enhancement of binding specificity for zinc finger binding domains has been described, for example, in U.S. Pat. No. 6,794,136.

In certain embodiments, the DNA-binding domain is part of a CRISPR/Cas nuclease system, including a single guide RNA (sgRNA) that binds to DNA. See, e.g., U.S. Pat. No. 8,697,359 and U.S. Pat. Publication Nos. 2015/0056705 and 2015/0159172. The CRISPR (clustered regularly interspaced short palindromic repeats) locus, which encodes RNA components of the system, and the cas (CRISPR-associated) locus, which encodes proteins (Jansen, et al. (2002) Mol. Microbiol. 43:1565-1575; Makarova, et al. (2002) Nucleic Acids Res. 30:482-496; Makarova, et al. (2006) Biol. Direct 1:7; Haft, et al. (2005) PLoS Comput. Biol. 1:e60) make up the gene sequences of the CRISPR/Cas nuclease system. CRISPR loci in microbial hosts contain a combination of CRISPR-associated (Cas) genes as well as non-coding RNA elements capable of programming the specificity of the CRISPR-mediated nucleic acid cleavage.

The Type II CRISPR is one of the most well characterized systems and carries out targeted DNA double-strand break in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA:tracrRNA complex directs functional domain (e.g., nuclease such as Cas) to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer. Activity of the CRISPR/Cas system comprises of three steps: (i) insertion of alien DNA sequences into the CRISPR array to prevent future attacks, in a process called ‘adaptation’, (ii) expression of the relevant proteins, as well as expression and processing of the array, followed by (iii) RNA-mediated interference with the alien nucleic acid. Thus, in the bacterial cell, several of the so-called ‘Cas’ proteins are involved with the natural function of the CRISPR/Cas system and serve roles in functions such as insertion of the alien DNA etc.

In certain embodiments, Cas protein may be a “functional derivative” of a naturally occurring Cas protein. A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The term “derivative” encompasses both amino acid sequence variants of polypeptide, covalent modifications, and fusions thereof such as derivative Cas proteins. Suitable derivatives of a Cas polypeptide or a fragment thereof include but are not limited to mutants, fusions, covalent modifications of Cas protein or a fragment thereof. Cas protein, which includes Cas protein or a fragment thereof, as well as derivatives of Cas protein or a fragment thereof, may be obtainable from a cell or synthesized chemically or by a combination of these two procedures. The cell may be a cell that naturally produces Cas protein, or a cell that naturally produces Cas protein and is genetically engineered to produce the endogenous Cas protein at a higher expression level or to produce a Cas protein from an exogenously introduced nucleic acid, which nucleic acid encodes a Cas that is same or different from the endogenous Cas. In some case, the cell does not naturally produce Cas protein and is genetically engineered to produce a Cas protein. In some embodiments, the Cas protein is a small Cas9 ortholog for delivery via an AAV vector (Ran, et al. (2015) Nature 520:186).

In some embodiments, the DNA binding domain is part of a TtAgo system (see Swarts, et al., ibid; Sheng, et al., ibid). In eukaryotes, gene silencing is mediated by the Argonaute (Ago) family of proteins. In this paradigm, Ago is bound to small (19-31 nt) RNAs. This protein-RNA silencing complex recognizes target RNAs via Watson-Crick base pairing between the small RNA and the target and endonucleolytically cleaves the target RNA (Vogel (2014) Science 344:972-973). In contrast, prokaryotic Ago proteins bind to small single-stranded DNA fragments and likely function to detect and remove foreign (often viral) DNA (Yuan, et al. (2005) Mol. Cell 19, 405; Olovnikov, et al. (2013) Mol. Cell 51:594; Swarts, et al., ibid). Exemplary prokaryotic Ago proteins include those from Aquifex aeolicus, Rhodobacter sphaeroides, and Thermus thermophilus.

One of the most well-characterized prokaryotic Ago protein is the one from T. thermophilus (TtAgo; Swarts, et al., ibid). TtAgo associates with either 15 nt or 13-25 nt single-stranded DNA fragments with 5′ phosphate groups. This “guide DNA” bound by TtAgo serves to direct the protein-DNA complex to bind a Watson-Crick complementary DNA sequence in a third-party molecule of DNA. Once the sequence information in these guide DNAs has allowed identification of the target DNA, the TtAgo-guide DNA complex cleaves the target DNA. Such a mechanism is also supported by the structure of the TtAgo-guide DNA complex while bound to its target DNA (G. Sheng et al., ibid). Ago from Rhodobacter sphaeroides (RsAgo) has similar properties (Olovnikov, et al., ibid).

Exogenous guide DNAs of arbitrary DNA sequence can be loaded onto the TtAgo protein (Swarts, et al., ibid.). Since the specificity of TtAgo cleavage is directed by the guide DNA, a TtAgo-DNA complex formed with an exogenous, investigator-specified guide DNA will therefore direct TtAgo target DNA cleavage to a complementary investigator-specified target DNA. In this way, one may create a targeted double-strand break in DNA. Use of the TtAgo-guide DNA system (or orthologous Ago-guide DNA systems from other organisms) allows for targeted cleavage of genomic DNA within cells. Such cleavage can be either single- or double-stranded. For cleavage of mammalian genomic DNA, it would be preferable to use of a version of TtAgo codon optimized for expression in mammalian cells. Further, it might be preferable to treat cells with a TtAgo-DNA complex formed in vitro where the TtAgo protein is fused to a cell-penetrating peptide. Further, it might be preferable to use a version of the TtAgo protein that has been altered via mutagenesis to have improved activity at 37° C. Ago-RNA-mediated DNA cleavage could be used to affect a panopoly of outcomes including gene knock-out, targeted gene addition, gene correction, targeted gene deletion using techniques standard in the art for exploitation of DNA breaks.

Thus, any DNA-binding domain can be used.

### Fusion Molecules

Fusion molecules comprising DNA-binding domains (e.g., ZFPs or TALEs, CRISPR/Cas components such as single guide RNAs) as described herein associated with a heterologous regulatory (functional) domain (or functional fragment thereof) are also provided. Common domains include, e.g., transcription factor domains (activators, repressors, co-activators, co-repressors), silencers, oncogenes (e.g., myc, jun, fos, myb, max, mad, rel, ets, bcl, myb, mos family members etc.); DNA repair enzymes and their associated factors and modifiers; DNA rearrangement enzymes and their associated factors and modifiers; chromatin associated proteins and their modifiers (e.g., kinases, acetylases and deacetylases); and DNA modifying enzymes (e.g., methyltransferases, topoisomerases, helicases, ligases, kinases, phosphatases, polymerases, endonucleases) and their associated factors and modifiers. Such fusion molecules include transcription factors comprising the DNA-binding domains described herein and a transcriptional regulatory domain as well as nucleases comprising the DNA-binding domains and one or more nuclease domains.

Suitable domains for achieving activation (transcriptional activation domains) include the HSV VP16 activation domain (see, e.g., Hagmann, et al. (1997) J. Virol. 71 :5952-5962) nuclear hormone receptors (see, e.g., Torchia, et al. (1998) Curr. Opin. Cell. Biol. 10:373-383); the p65 subunit of nuclear factor kappa B (Bitko & Barik (1998) J. Virol. 72:5610-5618 and Doyle & Hunt (1997) Neuroreport 8:2937-2942); Liu, et al. (1998) Cancer Gene Ther. 5:3-28), or artificial chimeric functional domains such as VP64 (Beerli, et al. (1998) Proc. Natl. Acad. Sci. USA 95: 14623-33), and degron (Molinari, et al. (1999) EMBO J. 18, 6439-6447). Additional exemplary activation domains include, Oct 1, Oct-2A, Sp1, AP-2, and CTF1 (Seipel, et al. (1992) EMBO J. 11, 4961-4968 as well as p300, CBP, PCAF, SRC1 PvALF, AtHD2A and ERF-2. See, for example, Robyr, et al. (2000) Mol. Endocrinol. 14:329-347; Collingwood, et al. (1999) J. Mol. Endocrinol. 23:255-275; Leo, et al. (2000) Gene 245:1-11; Manteuffel-Cymborowska (1999) Acta Biochim. Pol. 46:77-89; McKenna, et al. (1999) J. Steroid Biochem. Mol. Biol. 69:3-12; Malik, et al. (2000) Trends Biochem. Sci. 25:277-283; and Lemon, et al. (1999) Curr. Opin. Genet. Dev. 9:499-504. Additional exemplary activation domains include, but are not limited to, OsGAI, HALF-1, C1, AP1, ARF-5,-6,-7, and -8, CPRF1, CPRF4, MYC-RP/GP, and TRAB1. See, for example, Ogawa, et al. (2000) Gene 245:21-29; Okanami, et al. (1996) Genes Cells 1 :87-99; Goff, et al. (1991) Genes Dev. 5:298-309; Cho, et al. (1999) Plant Mol. Biol. 40:419-429; Ulmason, et al. (1999) Proc. Natl. Acad. Sci. USA 96:5844-5849; Sprenger-Haussels, et al. (2000) Plant J 22:1-8; Gong, et al. (1999) Plant Mol. Biol. 41:33-44; and Hobo, et al. (1999) Proc. Natl. Acad. Sci. USA 96:15,348-15,353.

It will be clear to those of skill in the art that, in the formation of a fusion protein (or a nucleic acid encoding same) between a DNA-binding domain and a functional domain, either an activation domain or a molecule that interacts with an activation domain is suitable as a functional domain. Essentially any molecule capable of recruiting an activating complex and/or activating activity (such as, for example, histone acetylation) to the target gene is useful as an activating domain of a fusion protein. Insulator domains, localization domains, and chromatin remodeling proteins such as ISWI-containing domains and/or methyl binding domain proteins suitable for use as functional domains in fusion molecules are described, for example, in U.S. Pat. No. 7,053,264.

Exemplary repression domains include, but are not limited to, KRAB A/B, KOX, TGF-beta-inducible early gene (TIEG), v-erbA, SID, MBD2, MBD3, members of the DNMT family (e.g., DNMT1, DNMT3A, DNMT3B), Rb, and MeCP2. See, for example, Bird, et al. (1999) Cell 99:451-454; Tyler, et al. (1999) Cell 99:443-446; Knoepfler, et al. (1999) Cell 99:447-450; and Robertson, et al. (2000) Nature Genet. 25:338-342. Additional exemplary repression domains include, but are not limited to, ROM2 and AtHD2A. See, for example, Chem, et al. (1996) Plant Cell 8:305-321; and Wu, et al. (2000) Plant J. 22:19-27.

Fusion molecules are constructed by methods of cloning and biochemical conjugation that are well known to those of skill in the art. Fusion molecules comprise a DNA-binding domain (e.g., ZFP, TALE, sgRNA) associated with a functional domain (e.g., a transcriptional activation or repression domain). Fusion molecules also optionally comprise nuclear localization signals (such as, for example, that from the SV40 medium T-antigen) and epitope tags (such as, for example, FLAG and hemagglutinin). Fusion proteins (and nucleic acids encoding them) are designed such that the translational reading frame is preserved among the components of the fusion.

Fusions between a polypeptide component of a functional domain (or a functional fragment thereof) on the one hand, and a non-protein DNA-binding domain (e.g., antibiotic, intercalator, minor groove binder, nucleic acid) on the other, are constructed by methods of biochemical conjugation known to those of skill in the art. See, for example, the Pierce Chemical Company (Rockford, IL) Catalogue. Methods and compositions for making fusions between a minor groove binder and a polypeptide have been described. Mapp, et al. (2000) Proc. Natl. Acad. Sci. USA 97:3930-3935. Furthermore, single guide RNAs of the CRISPR/Cas system associate with functional domains to form active transcriptional regulators and nucleases.

In certain embodiments, the target site is present in an accessible region of cellular chromatin. Accessible regions can be determined as described, for example, in U.S. Pat. Nos. 7,217,509 and 7,923,542. If the target site is not present in an accessible region of cellular chromatin, one or more accessible regions can be generated as described in U.S. Pat. Nos. 7,785,792 and 8,071,370. In additional embodiments, the DNA-binding domain of a fusion molecule is capable of binding to cellular chromatin regardless of whether its target site is in an accessible region or not. For example, such DNA-binding domains are capable of binding to linker DNA and/or nucleosomal DNA. Examples of this type of “pioneer” DNA binding domain are found in certain steroid receptor and in hepatocyte nuclear factor 3 (HNF3) (Cordingley, et al. (1987) Cell 48:261-270; Pina, et al. (1990) Cell 60:719-731; and Cirillo, et al. (1998) EMBO J. 17:244-254).

The fusion molecule may be formulated with a pharmaceutically acceptable carrier, as is known to those of skill in the art. See, for example, Remington’s Pharmaceutical Sciences, 17th ed., 1985; and U.S. Pat. Nos. 6,453,242 and 6,534,261.

The functional component/domain of a fusion molecule can be selected from any of a variety of different components capable of influencing transcription of a gene once the fusion molecule binds to a target sequence via its DNA binding domain. Hence, the functional component can include, but is not limited to, various transcription factor domains, such as activators, repressors, co-activators, co-repressors, and silencers.

Additional exemplary functional domains are disclosed, for example, in U.S. Pat. Nos. 6,534,261 and 6,933,113.

Functional domains that are regulated by exogenous small molecules or ligands may also be selected. For example, RheoSwitch® technology may be employed wherein a functional domain only assumes its active conformation in the presence of the external RheoChem™ ligand (see for example U.S. Pat. Publication No. 2009/0136465). Thus, the ZFP may be operably linked to the regulatable functional domain wherein the resultant activity of the ZFP-TF is controlled by the external ligand.

### Nucleases

In certain embodiments, the fusion molecule comprises a DNA-binding binding domain associated with a cleavage (nuclease) domain. As such, gene modification can be achieved using a nuclease, for example an engineered nuclease. Engineered nuclease technology is based on the engineering of naturally occurring DNA-binding proteins. For example, engineering of homing endonucleases with tailored DNA-binding specificities has been described. Chames, et al. (2005) Nucleic Acids Res 33(20):e178; Arnould, et al. (2006) J. Mol. Biol. 355:443-458. In addition, engineering of ZFPs has also been described. See, e.g., U.S. Pat. Nos. 6,534,261; 6,607,882; 6,824,978; 6,979,539; 6,933,113; 7,163,824; and 7,013,219.

In addition, ZFPs and/or TALEs can be fused to nuclease domains to create ZFNs and TALENs - a functional entity that is able to recognize its intended nucleic acid target through its engineered (ZFP or TALE) DNA binding domain and cause the DNA to be cut near the DNA binding site via the nuclease activity.

Thus, the methods and compositions described herein are broadly applicable and may involve any nuclease of interest. Non-limiting examples of nucleases include meganucleases, TALENs and zinc finger nucleases. The nuclease may comprise heterologous DNA-binding and cleavage domains (e.g., zinc finger nucleases; meganuclease DNA-binding domains with heterologous cleavage domains) or, alternatively, the DNA-binding domain of a naturally-occurring nuclease may be altered to bind to a selected target site (e.g., a meganuclease that has been engineered to bind to site different than the cognate binding site).

In any of the nucleases described herein, the nuclease can comprise an engineered TALE DNA-binding domain and a nuclease domain (e.g., endonuclease and/or meganuclease domain), also referred to as TALENs. Methods and compositions for engineering these TALEN proteins for robust, site specific interaction with the target sequence of the user’s choosing have been published (see U.S. Pat. No. 8,586,526). In some embodiments, the TALEN comprises an endonuclease (e.g., FokI) cleavage domain or cleavage half-domain. In other embodiments, the TALE-nuclease is a mega TAL. These mega TAL nucleases are fusion proteins comprising a TALE DNA binding domain and a meganuclease cleavage domain. The meganuclease cleavage domain is active as a monomer and does not require dimerization for activity. (See Boissel, et al. (2013) Nucl Acid Res: 1-13, doi: 10.1093/nar/gkt1224). In addition, the nuclease domain may also exhibit DNA-binding functionality.

In still further embodiments, the nuclease comprises a compact TALEN (cTALEN). These are single chain fusion proteins linking a TALE DNA binding domain to a TevI nuclease domain. The fusion protein can act as either a nickase localized by the TALE region, or can create a double strand break, depending upon where the TALE DNA binding domain is located with respect to the TevI nuclease domain (see Beurdeley, et al. (2013) Nat Comm: 1-8 DOI: 10.1038/ncomms2782). Any TALENs may be used in combination with additional TALENs (e.g., one or more TALENs (cTALENs or FokI-TALENs) with one or more mega-TALs) or other DNA cleavage enzymes.

In certain embodiments, the nuclease comprises a meganuclease (homing endonuclease) or a portion thereof that exhibits cleavage activity. Naturally-occurring meganucleases recognize 15-40 base-pair cleavage sites and are commonly grouped into four families: the LAGLIDADG family (“LAGLIDADG” disclosed as SEQ ID NO:122), the GIY-YIG family, the His-Cyst box family and the HNH family. Exemplary homing endonucleases include I-SceI, I-CeuI, PI-PspI, PI-Sce, I-SceIV, I-CsmI, I-PanI, I-SceII, I-PpoI, I-SceIII, I-Crel, I-TevI, I-TevII and I-TevIII. Their recognition sequences are known. See also U.S. Pat. No. 5,420,032; U.S. Pat. No. 6,833,252; Belfort, et al. (1997) Nucleic Acids Res. 25:3379-3388; Dujon, et al. (1989) Gene 82:115-118; Perler, et al. (1994) Nucleic Acids Res. 22:1125-1127; Jasin (1996) Trends Genet. 12:224-228; Gimble, et al. (1996) J. Mol. Biol. 263:163-180; Argast, et al. (1998) J. Mol. Biol. 280:345-353 and the New England Biolabs catalogue.

DNA-binding domains from naturally-occurring meganucleases, primarily from the LAGLIDADG family (“LAGLIDADG” disclosed as SEQ ID NO:122), have been used to promote site-specific genome modification in plants, yeast, Drosophila, mammalian cells and mice, but this approach has been limited to the modification of either homologous genes that conserve the meganuclease recognition sequence (Monet, et al. (1999), Biochem. Biophysics. Res. Common. 255: 88-93) or to pre-engineered genomes into which a recognition sequence has been introduced (Route, et al. (1994), Mol. Cell. Biol. 14:8096-106; Chilton, et al. (2003), Plant Physiology. 133:956-65; Puchta, et al. (1996), Proc. Natl. Acad. Sci. USA 93:5055-60; Rong, et al. (2002), Genes Dev. 16:1568-81; Gouble, et al. (2006), J. Gene Med. 8(5):616-622). Accordingly, attempts have been made to engineer meganucleases to exhibit novel binding specificity at medically or biotechnologically relevant sites (Porteus, et al. (2005), Nat. Biotechnol. 23:967-73; Sussman, et al. (2004), J. Mol. Biol. 342:31-41; Epinat, et al. (2003) Nucleic Acids Res. 31:2952-62; Chevalier, et al. (2002) Molec. Cell 10:895-905; Epinat, et al. (2003) Nucleic Acids Res. 31:2952-2962; Ashworth, et al. (2006) Nature 441:656-659; Paques, et al. (2007) Current Gene Therapy 7:49-66; U.S. Pat. Publication Nos. 2007/0117128; 2006/0206949; 2006/0153826; 2006/0078552; and 2004/0002092). In addition, naturally-occurring or engineered DNA-binding domains from meganucleases can be operably linked with a cleavage domain from a heterologous nuclease (e.g., FokI) and/or cleavage domains from meganucleases can be operably linked with a heterologous DNA-binding domain (e.g., ZFP or TALE).

In other embodiments, the nuclease is a zinc finger nuclease (ZFN) or TALE DNA binding domain-nuclease fusion (TALEN). ZFNs and TALENs comprise a DNA binding domain (zinc finger protein or TALE DNA binding domain) that has been engineered to bind to a target site in a gene of choice and cleavage domain or a cleavage half-domain (e.g., from a restriction and/or meganuclease as described herein).

As described in detail above, zinc finger binding domains and TALE DNA binding domains can be engineered to bind to a sequence of choice. See, for example, Beerli, et al. (2002) Nature Biotechnol. 20:135-141; Pabo, et al. (2001) Ann. Rev. Biochem. 70:313-340; Isalan, et al. (2001) Nature Biotechnol. 19:656-660; Segal, et al. (2001) Curr. Opin. Biotechnol. 12:632-637; Choo, et al. (2000) Curr. Opin. Struct. Biol. 10:411-416. An engineered zinc finger binding domain or TALE protein can have a novel binding specificity, compared to a naturally-occurring protein. Engineering methods include, but are not limited to, rational design and various types of selection. Rational design includes, for example, using databases comprising triplet (or quadruplet) nucleotide sequences and individual zinc finger or TALE amino acid sequences, in which each triplet or quadruplet nucleotide sequence is associated with one or more amino acid sequences of zinc fingers or TALE repeat units which bind the particular triplet or quadruplet sequence. See, for example, U.S. Pat. Nos. 6,453,242 and 6,534,261, incorporated by reference herein in their entireties. In certain embodiments, the DNA-binding domains comprise ZFPs derived from (e.g., the ZFP component) of the ZFNs designated 68957, 72678, 72732, 72748 (B2M) or 68846 (TCR).

Selection of target sites; and methods for design and construction of fusion proteins (and polynucleotides encoding same) are known to those of skill in the art and described in detail in U.S. Pat. Nos. 7,888,121 and 8,409,861, incorporated by reference in their entireties herein.

In addition, as disclosed in these and other references, zinc finger domains, TALEs and/or multi-fingered zinc finger proteins may be linked together using any suitable linker sequences, including for example, linkers of 5 or more amino acids in length. See, e.g., U.S. Pat. Nos. 6,479,626; 6,903,185; and 7,153,949 for exemplary linker sequences 6 or more amino acids in length. The proteins described herein may include any combination of suitable linkers between the individual zinc fingers of the protein. See, also, U.S. Pat. No. 8,772,453.

Thus, nucleases such as ZFNs, TALENs and/or meganucleases can comprise any DNA-binding domain and any nuclease (cleavage) domain (cleavage domain, cleavage half-domain). As noted above, the cleavage domain may be heterologous to the DNA-binding domain, for example a zinc finger or TAL-effector DNA-binding domain and a cleavage domain from a nuclease or a meganuclease DNA-binding domain and cleavage domain from a different nuclease. Heterologous cleavage domains can be obtained from any endonuclease or exonuclease. Exemplary endonucleases from which a cleavage domain can be derived include, but are not limited to, restriction endonucleases and homing endonucleases. See, for example, 2002-2003 Catalogue, New England Biolabs, Beverly, MA; and Belfort, et al. (1997) Nucleic Acids Res. 25:3379-3388. Additional enzymes which cleave DNA are known (e.g., S1 Nuclease; mung bean nuclease; pancreatic DNase I; micrococcal nuclease; yeast HO endonuclease; see also Linn, et al. (eds.) Nucleases, Cold Spring Harbor Laboratory Press, 1993). One or more of these enzymes (or functional fragments thereof) can be used as a source of cleavage domains and cleavage half-domains.

Similarly, a cleavage half-domain can be derived from any nuclease or portion thereof, as set forth above, that requires dimerization for cleavage activity. In general, two fusion proteins are required for cleavage if the fusion proteins comprise cleavage half-domains. Alternatively, a single protein comprising two cleavage half-domains can be used. The two cleavage half-domains can be derived from the same endonuclease (or functional fragments thereof), or each cleavage half-domain can be derived from a different endonuclease (or functional fragments thereof). In addition, the target sites for the two fusion proteins are preferably disposed, with respect to each other, such that binding of the two fusion proteins to their respective target sites places the cleavage half-domains in a spatial orientation to each other that allows the cleavage half-domains to form a functional cleavage domain, e.g., by dimerizing. Thus, in certain embodiments, the near edges of the target sites are separated by 5-8 nucleotides or by 15-18 nucleotides. However, any integral number of nucleotides or nucleotide pairs can intervene between two target sites (e.g., from 2 to 50 nucleotide pairs or more). In general, the site of cleavage lies between the target sites, but may lie 1 or more kilobases away from the cleavage site, including between 1-50 base pairs (or any value therebetween including 1-5, 1-10, and 1-20 base pairs), 1-100 base pairs (or any value therebetween), 100-500 base pairs (or any value therebetween), 500 to 1000 base pairs (or any value therebetween) or even more than 1 kb from the cleavage site.

Restriction endonucleases (restriction enzymes) are present in many species and are capable of sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme FokI catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li, et al. (1992) Proc. Natl. Acad. Sci. USA 89:4275-4279; Li, et al. (1993) Proc. Natl. Acad. Sci. USA 90:2764-2768; Kim, et al. (1994a) Proc. Natl. Acad. Sci. USAy 91:883-887; Kim, et al. (1994b) J. Biol. Chem. 269:31,978-31,982. Thus, in one embodiment, fusion proteins comprise the cleavage domain (or cleavage half-domain) from at least one Type IIS restriction enzyme and one or more zinc finger binding domains, which may or may not be engineered.

An exemplary Type IIS restriction enzyme, whose cleavage domain is separable from the binding domain, is FokI. This particular enzyme is active as a dimer. Bitinaite, et al. (1998) Proc. Natl. Acad. Sci. USA 95: 10,570-10,575. The sequence of the full-length FokI is shown below. The cleavage domain used in the nucleases described herein is shown in italics and underlining (positions 384 to 579 of the full-length protein) where the holo protein sequence is described below (SEQ ID NO: 138):

MVSKIRTFGWVQNPGKFENLKRVVQVFDRNSKVHNEVKNIKIPTLVKESK
IQKELVAIMNQHDLIYTYKELVGTGTSIRSEAPCDAIIQATIADQGNKKG
YIDNWSSDGFLRWAHALGFIEYINKSDSFVITDVGLAYSKSADGSAIEKE
ILIEAISSYPPAIRILTLLEDGQHLTKFDLGKNLGFSGESGFTSLPEGIL
LDTLANAMPKDKGEIRNNWEGSSDKYARMIGGWLDKLGLVKQGKKEFIIP
TLGKPDNKEFISHAFKITGEGLKVLRRAKGSTKFTRVPKRVYWEMLATNL
TDKEYVRTRRALILEILIKAGSLKIEQIQDNLKKLGFDEVIETIENDIKG
LINTGIFIEIKGRFYQLKDHILQFVIPNRGVTKOLVKSELEEKKSELRHK LKYVPHEYIELIEIARNSTQDRILEMKVMEFFMKVYGYRGKHLGGSRKPD GAIYTVGSPIDYGVIVDTKAYSGGYNLPIGQADEMQRYVEENQTRNKHIN PNEWWKVYP SSVTEFKFLFVSGHFKGNYKAQLTRLNHITNCNGAVLSVEE LLIGGEMIKAGTLTLEEVRRKFNNGEINF (SEQ ID NO: 138)

Accordingly, for the purposes of the present disclosure, the portion of the FokI enzyme used in the disclosed fusion proteins is considered a cleavage half-domain. Thus, for targeted double-stranded cleavage and/or targeted replacement of cellular sequences using zinc finger-FokI fusions, two fusion proteins, each comprising a FokI cleavage half-domain, can be used to reconstitute a catalytically active cleavage domain. Alternatively, a single polypeptide molecule containing a zinc finger binding domain and two FokI cleavage half-domains can also be used. Parameters for targeted cleavage and targeted sequence alteration using zinc finger-FokI fusions are provided elsewhere in this disclosure.

A cleavage domain or cleavage half-domain can be any portion of a protein that retains cleavage activity, or that retains the ability to multimerize (e.g., dimerize) to form a functional cleavage domain.

Exemplary Type IIS restriction enzymes are described in International Patent Publication No. WO 07/014275, incorporated herein in its entirety. Additional restriction enzymes also contain separable binding and cleavage domains, and these are contemplated by the present disclosure. See, for example, Roberts, et al. (2003) Nucleic Acids Res. 31:418-420.

In certain embodiments, the cleavage domain comprises one or more engineered cleavage half-domain (also referred to as dimerization domain mutants) that minimize or prevent homodimerization, as described, for example, in U.S. Pat. Nos. 7,914,796; 8,034,598; and 8,623,618; and U.S. Pat. Publication No. 2011/0201055, the disclosures of all of which are incorporated by reference in their entireties herein. “Sharkey” mutations (e.g., 418 and 441, numbered relative to full-length) and additional mutations, for example, to residue 416 (e.g., R416S) and/or residue 525 (e.g., K525S) as described in U.S. Pat. Publication No. 2018/0087072, may also be included. Thus, the FokI cleavage domains used in the nucleases of the invention may be mutated at one or more of the following amino acid residues positions (numbered relative to full length): 416, 418, 441, 446, 447, 479, 483, 484, 486, 487, 490, 491, 496, 498, 499, 500, 525, 531, 534, 537, and/or 538.

Exemplary engineered cleavage half-domains of FokI that form obligate heterodimers include a pair in which a first cleavage half-domain includes mutations at amino acid residues at positions 490 and 538 of FokI and a second cleavage half-domain includes mutations at amino acid residues 486 and 499.

Thus, in one embodiment, a mutation at 490 replaces Glu (E) with Lys (K); the mutation at 538 replaces Iso (I) with Lys (K); the mutation at 486 replaced Gln (Q) with Glu (E); and the mutation at position 499 replaces Iso (I) with Lys (K). Specifically, the engineered cleavage half-domains described herein were prepared by mutating positions 490 (E→K) and 538 (I→K) in one cleavage half-domain to produce an engineered cleavage half-domain designated “E490K:I538K” and by mutating positions 486 (Q→E) and 499 (I→L) in another cleavage half-domain to produce an engineered cleavage half-domain designated “Q486E:I499L”. The engineered cleavage half-domains described herein are obligate heterodimer mutants in which aberrant cleavage is minimized or abolished. See, e.g., U.S. Pat. Nos. 7,914,796 and 8,034,598, the disclosures of which are incorporated by reference in their entireties for all purposes. In certain embodiments, the engineered cleavage half-domain comprises mutations at positions 486, 499 and 496 (numbered relative to wild-type FokI), for instance mutations that replace the wild type Gln (Q) residue at position 486 with a Glu (E) residue, the wild type Iso (I) residue at position 499 with a Leu (L) residue and the wild-type Asn (N) residue at position 496 with an Asp (D) or Glu (E) residue (also referred to as a “ELD” and “ELE” domains, respectively). In other embodiments, the engineered cleavage half-domain comprises mutations at positions 490, 538 and 537 (numbered relative to wild-type FokI), for instance mutations that replace the wild type Glu (E) residue at position 490 with a Lys (K) residue, the wild type Iso (I) residue at position 538 with a Lys (K) residue, and the wild-type His (H) residue at position 537 with a Lys (K) residue or a Arg (R) residue (also referred to as “KKK” and “KKR” domains, respectively). In other embodiments, the engineered cleavage half-domain comprises mutations at positions 490 and 537 (numbered relative to wild-type FokI), for instance mutations that replace the wild type Glu (E) residue at position 490 with a Lys (K) residue and the wild-type His (H) residue at position 537 with a Lys (K) residue or a Arg (R) residue (also referred to as “KIK” and “KIR” domains, respectively).

In other embodiments, the engineered cleavage half-domain comprises mutations at positions 487, 499 and 496 (numbered relative to wild-type FokI), for instance mutations that replace the wild-type Arg (R) residue at position 487 with an Asp (D) residue and the wild-type Ile (I) residue at position 499 with an Ala (A) and the wild-type Asn (N) residue at position 496 with an Asp (D) residue (also referred to as “DAD”) and/or mutations at positions 483, 538 and 537 (numbered relative to wild-type FokI), for instance, mutations that replace the wild-type Asp (D) residue at position 483 with an Arg (R) residue and the wild-type Ile (I) residue at position 538 with a Val (V) residue, and the wild-type His (H) residue at position 537 with an Arg (R) residue (also referred to as “RVR”). See, e.g., U.S. Pat. Nos. 8,962,281; 7,914,796; 8,034,598; and 8,623,618, the disclosures of which are incorporated by reference in its entirety for all purposes. In other embodiments, the engineered cleavage half domain comprises the “Sharkey” and/or “Sharkey” mutations (see Guo, et al. (2010) J. Mol. Biol. 400(1):96-107).

Thus, non-limiting examples of FokI domains that can be used in the nucleases described herein include: Fok mutants shown in Table 8 (e.g., ELD, KKR, etc.), FokI-Sharkey (S418P+K441E), FokI ELD (Q->E at position 486, I->L at 499, N->D at position 496), FokI ELD, Sharkey (Q->E at position 486, I->L at position 499, N->D at position 496, S418P+K441E), FokI ELD, R416E (Q->E at position 486, I->L at position 499, N->D at position 496, R416E), FokI ELD, Sharkey, R416E (Q->E at position 486, I->L at position 499, N->D at position 496, S418P+K441E, R416E), FokI ELD, R416Y (Q->E at position 486, I->L at position 499, N->D at position 496, R416Y), FokI ELD, Sharkey, R416E (Q->E at position 486, I->L at position 499, N->D at position 496, S418P+K441E, R416E), FokI ELD, S418E (Q->E at position 486, I->L at position 499, N->D at position 496, S418E), FokI ELD, Sharkey partial, S418E (Q->E at position 486, I->L at position 499, N->D at position 496, K441E, S418E), FokI ELD, K525S (Q->E at position 486, I->L at position 499, N->D at position 496, K525S), FokI ELD, Sharkey K525S (Q->E at position 486, I->L at position 499, N->D at position 496, S418P+K441E, K525S), FokI ELD, I479T (Q->E at position 486, I->L at position 499, N->D at position 496, I479T), FokI ELD, Sharkey, I479T (Q->E at position 486, I->L at position 499, N->D at position 496, S418P+K441E, I479T), FokI ELD, P478D (Q->E at position 486, I->L at position 499, N->D at position 496, P478D), FokI ELD, Sharkey, P478D (Q->E at position 486, I->L at position 499, N->D at position 496, S418P+K441E, P478D), FokI ELD, Q481D (Q->E at position 486, I->L at position 499, N->D at position 496, Q481D), FokI ELD, Sharkey, Q481D (Q->E at position 486, I->L at position 499, N->D at position 496, S418P+K441E, Q481D), FokI KKR (E->K at position 490, I->K at position 538, H->R at position 537), FokI KKR Sharkey, (E->K at position 490, I->K at position 538, H->R at position 537, S418P+K441E), FokI KKR, Q481E (E->K at position 490, I->K at position 538, H->R at position 537, Q481E), FokI KKR, Sharkey Q481E (E->K at position 490, I->K at position 538, H->R at position 537, S418P+K441E, Q481E), FokI KKR, R416E (E->K at position 490, I->K at position 538, H->R at position 537, R416E), FokI KKR, Sharkey, R416E (E->K at position 490, I->K at position 538, H->R at position 537, S418P+K441E, R416E), FokI KKR, K525S (E->K at position 490, I->K at position 538, H->R at position 537, K525S), FokI KKR, Sharkey, K525S (E->K at position 490, I->K at position 538, H->R at position 537, S418P+K441E, K525S), FokI KKR, R416Y (E->K at position 490, I->K position 538, H->R at position 537, R416Y), FokI KKR, Sharkey, R416Y (E->K at position 490, I->K at position 538, H->R at position 537, S418P+K441E, R416Y), FokI, KKR I479T (E->K at position 490, I->K at position 538, H->R at position 537, I479T), FokI, KKR Sharkey I479T (E->K at position 490, I->K at position 538, H->R at position 537, S418P+K441E, I479T, FokI, KKR P478D(E->K at position 490, I->K at positions 538, H->R at position 537, P478D), FokI, KKR Sharkey P478D(E->K at position 490, I->K at position 538, H->R at position 537, P478D), FokI DAD (R->D at position 487, N->D at position 496, I->A at position 499), FokI DAD Sharkey (R->D at position 487, N->D at position 496, I->A at position 499, S418P+K441E), FokI RVR (D->R at position 483, H->R at position 537, I->V at position 538), FokI RVR Sharkey (D->R at position 483, H->R at position 537, I->V at position 538, S418P+K441E).

The ZFNs described herein may also include any linker sequence, including but not limited to sequences disclosed herein (L0, N7a, N7c, etc.) and/or those disclosed in U.S. Pat. No. 7,888,121; 7,914,796; 8,034,598; 8,623,618; 9,567,609; and U.S. Publication No. 20170218349, which may be used between the N- or C-terminal of the DNA-binding domain and N- or C-terminal of the FokI cleavage domain.

ZFPs of the ZFNs as described herein (including engineered and/or wild-type cleavage domains) may also include modifications to increase the specificity of a ZFN, including a nuclease pair, for its intended target relative to other unintended cleavage sites, known as off-target sites (see U.S. Pat. Publication No. 20180087072). Thus, nucleases described herein can comprise specific linkers between the DNA-binding domain and cleavage domain; and/or can comprise mutations in one or more of their DNA binding domain backbone regions and/or one or more mutations in their nuclease cleavage domains as described above. The ZFPs of these nucleases can include mutations to amino acids within the ZFP DNA binding domain (‘ZFP backbone’) that can interact non-specifically with phosphates on the DNA backbone, but they do not comprise changes in the DNA recognition helices. Thus, the invention includes ZFPs comprising mutations of cationic amino acid residues in the ZFP backbone that are not required for nucleotide target specificity. In some embodiments, these mutations in the ZFP backbone comprise mutating a cationic amino acid residue to a neutral or anionic amino acid residue. In some embodiments, these mutations in the ZFP backbone comprise mutating a polar amino acid residue to a neutral or non-polar amino acid residue. In preferred embodiments, mutations at made at position (-5), (-9) and/or position (-14) relative to the DNA binding helix. In some embodiments, a zinc finger may comprise one or more mutations at (-5), (-9) and/or (-14). In further embodiments, one or more zinc finger in a multi-finger zinc finger protein may comprise mutations in (-5), (-9) and/or (-14). In some embodiments, the amino acids at (-5), (-9) and/or (-14) (e.g., an arginine (R) or lysine (K)) are mutated to an alanine (A), leucine (L), Ser (S), Asp (N), Glu (E), Tyr (Y) and/or glutamine (Q).

In certain embodiments, the ZFNs comprise at least one of the following pairs: 68796 and 68813; 68796 and 68861; 68812 and 68813; 68876 and 68877; 68815 and 55266; 68879 and 55266; 68798 and 68815; or 68846 and 53853 as shown in Table 6. In other embodiments, the ZFNs comprise at least one of the following pairs: 57531 and 72732; 57531 and 72748; 68957 and 57071; 68957 and 72732; 68957 and 72748; 72678 and 57071; 72678 and 72732; or 72678 and 72748 as shown in Table 8.

Alternatively, nucleases may be assembled in vivo at the nucleic acid target site using so-called “split-enzyme” technology (see, e.g., U.S. Pat. Publication No. 2009/0068164). Components of such split enzymes may be expressed either on separate expression constructs or can be linked in one open reading frame where the individual components are separated, for example, by a self-cleaving 2A peptide or IRES sequence. Components may be individual zinc finger binding domains or domains of a meganuclease nucleic acid binding domain.

Nucleases (e.g., ZFNs and/or TALENs) can be screened for activity prior to use, for example in a yeast-based chromosomal system as described in as described in U.S. Pat. No. 8,563,314.

In certain embodiments, the nuclease comprises a CRISPR/Cas system. The CRISPR (clustered regularly interspaced short palindromic repeats) locus, which encodes RNA components of the system, and the Cas (CRISPR-associated) locus, which encodes proteins (Jansen, et al. (2002) Mol. Microbiol. 43:1565-1575; Makarova, et al. (2002) Nucleic Acids Res. 30:482-496; Makarova, et al. (2006) Biol. Direct 1:7; Haft, et al. (2005) PLoS Comput. Biol. 1: e60) make up the gene sequences of the CRISPR/Cas nuclease system. CRISPR loci in microbial hosts contain a combination of CRISPR-associated (Cas) genes as well as non-coding RNA elements capable of programming the specificity of the CRISPR-mediated nucleic acid cleavage.

The Type II CRISPR is one of the most well characterized systems and carries out targeted DNA double-strand break in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA:tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer. Activity of the CRISPR/Cas system comprises of three steps: (i) insertion of alien DNA sequences into the CRISPR array to prevent future attacks, in a process called ‘adaptation’, (ii) expression of the relevant proteins, as well as expression and processing of the array, followed by (iii) RNA-mediated interference with the alien nucleic acid. Thus, in the bacterial cell, several of the so-called ‘Cas’ proteins are involved with the natural function of the CRISPR/Cas system and serve roles in functions such as insertion of the alien DNA etc.

In certain embodiments, Cas protein may be a “functional derivative” of a naturally occurring Cas protein. A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The term “derivative” encompasses both amino acid sequence variants of polypeptide, covalent modifications, and fusions thereof. Suitable derivatives of a Cas polypeptide or a fragment thereof include but are not limited to mutants, fusions, covalent modifications of Cas protein or a fragment thereof. Cas protein, which includes Cas protein or a fragment thereof, as well as derivatives of Cas protein or a fragment thereof, may be obtainable from a cell or synthesized chemically or by a combination of these two procedures. The cell may be a cell that naturally produces Cas protein, or a cell that naturally produces Cas protein and is genetically engineered to produce the endogenous Cas protein at a higher expression level or to produce a Cas protein from an exogenously introduced nucleic acid, which nucleic acid encodes a Cas that is same or different from the endogenous Cas. In some case, the cell does not naturally produce Cas protein and is genetically engineered to produce a Cas protein.

Exemplary CRISPR/Cas nuclease systems targeted to TCR genes and other genes are disclosed for example, in U.S. Pat. Publication No. 2015/0056705. The nuclease(s) may make one or more double-stranded and/or single-stranded cuts in the target site. In certain embodiments, the nuclease comprises a catalytically inactive cleavage domain (e.g., FokI and/or Cas protein). See, e.g., U.S. Pat. Nos. 9,200,266 and 8,703,489 and Guillinger, et al. (2014) Nature Biotech. 32(6):577-582. The catalytically inactive cleavage domain may, in combination with a catalytically active domain act as a nickase to make a single-stranded cut. Therefore, two nickases can be used in combination to make a double-stranded cut in a specific region. Additional nickases are also known in the art, for example, McCaffrey, et al. (2016) Nucleic Acids Res. 44(2):e11. doi: 10.1093/nar/gkv878. Epub 2015 Oct 19. In addition, dead Cas (‘dCas’) or a Cas nickase may be fused to a base modifying enzyme (e.g., cytidine deaminase) to create a base editing system (Komor, et al. (2016) Nature 533:420). These systems allow for the alteration of a DNA base (modification) by the base editor complex without creating a double strand break in the DNA. Thus, in some embodiments, guide RNAs (Table 2) may be used to introduce mutations in a TRAC gene to cause a knock out.

### Delivery

The proteins (e.g., transcription factors, nucleases, TCR and CAR molecules), polynucleotides and/or compositions comprising the proteins and/or polynucleotides described herein may be delivered to a target cell by any suitable means, including, for example, by injection of the protein and/or mRNA components. In some embodiments, the proteins are introduced into the cell by cell squeezing (see Kollmannsperger, et al. (2016) Nat Comm 7, 10372 doi:10.1038/ncomms10372).

Suitable cells include but not limited to eukaryotic and prokaryotic cells and/or cell lines. Non-limiting examples of such cells or cell lines generated from such cells include T-cells, COS, CHO (e.g., CHO-S, CHO-K1, CHO-DG44, CHO-DUXB11, CHO-DUKX, CHOK1SV), VERO, MDCK, WI38, V79, B14AF28-G3, BHK, HaK, NS0, SP2/0-Ag14, HeLa, HEK293 (e.g., HEK293-F, HEK293-H, HEK293-T), and perC6 cells as well as insect cells such as Spodoptera fugiperda (Sf), or fungal cells such as Saccharomyces, Pichia and Schizosaccharomyces. In certain embodiments, the cell line is a CHO-K1, MDCK or HEK293 cell line. Suitable cells also include stem cells such as, by way of example, embryonic stem cells, induced pluripotent stem cells (iPS cells), hematopoietic stem cells, neuronal stem cells and mesenchymal stem cells.

Methods of delivering proteins comprising DNA-binding domains as described herein are described, for example, in U.S. Pat. Nos. 6,453,242; 6,503,717; 6,534,261; 6,599,692; 6,607,882; 6,689,558; 6,824,978; 6,933,113; 6,979,539; 7,013,219; and 7,163,824, the disclosures of all of which are incorporated by reference herein in their entireties.

DNA binding domains and fusion proteins comprising these DNA binding domains as described herein may also be delivered using vectors containing sequences encoding one or more of the DNA-binding protein(s). Additionally, additional nucleic acids (e.g., donors) also may be delivered via these vectors. Any vector systems may be used including, but not limited to, plasmid vectors, retroviral vectors, lentiviral vectors, adenovirus vectors, poxvirus vectors; herpesvirus vectors and adeno-associated virus vectors, etc. See, also, U.S. Pat. Nos. 6,534,261; 6,607,882; 6,824,978; 6,933,113; 6,979,539; 7,013,219; and 7,163,824, incorporated by reference herein in their entireties. Furthermore, it will be apparent that any of these vectors may comprise one or more DNA-binding protein-encoding sequences and/or additional nucleic acids as appropriate. Thus, when one or more DNA-binding proteins as described herein are introduced into the cell, and additional DNAs as appropriate, they may be carried on the same vector or on different vectors. When multiple vectors are used, each vector may comprise a sequence encoding one or multiple DNA-binding proteins and additional nucleic acids as desired.

Conventional viral and non-viral based gene transfer methods can be used to introduce nucleic acids encoding engineered DNA-binding proteins in cells (e.g., mammalian cells) and target tissues and to co-introduce additional nucleotide sequences as desired. Such methods can also be used to administer nucleic acids (e.g., encoding DNA-binding proteins and/or donors) to cells in vitro. In certain embodiments, nucleic acids are administered for in vivo or ex vivo gene therapy uses. Non-viral vector delivery systems include DNA plasmids, naked nucleic acid, and nucleic acid complexed with a delivery vehicle such as a liposome, lipid nanoparticle or poloxamer. Viral vector delivery systems include DNA and RNA viruses, which have either episomal or integrated genomes after delivery to the cell. For a review of gene therapy procedures, see Anderson (1992) Science 256:808-813; Nabel & Felgner (1993) TIBTECH 11:211-217; Mitani & Caskey (1993) TIBTECH 11:162-166; Dillon (1993) TIBTECH 11:167-175; Miller (1992) Nature 357:455-460; Van Brunt (1988) Biotechnology 6(10):1149-1154; Vigne (1995) Restorative Neurology and Neuroscience 8:35-36; Kremer & Perricaudet (1995) British Medical Bulletin 51(1):31-44; Haddada, et al. (1995) Current Topics in Microbiology and Immunology Doerfler and Böhm (eds.); and Yu, et al. (1994) Gene Therapy 1:13-26.

Methods of non-viral delivery of nucleic acids include electroporation, lipofection, microinjection, biolistics, virosomes, liposomes, lipid nanoparticles, immunoliposomes, polycation or lipid:nucleic acid conjugates, naked DNA, mRNA, artificial virions, and agent-enhanced uptake of DNA. Sonoporation using, e.g., the Sonitron 2000 system (Rich-Mar) can also be used for delivery of nucleic acids. In a preferred embodiment, one or more nucleic acids are delivered as mRNA. Also preferred is the use of capped mRNAs to increase translational efficiency and/or mRNA stability. Especially preferred are ARCA (anti-reverse cap analog) caps or variants thereof. See U.S. Pat. Nos. 7,074,596 and 8,153,773, incorporated by reference herein.

Additional exemplary nucleic acid delivery systems include those provided by Amaxa Biosystems (Cologne, Germany), Maxcyte, Inc. (Rockville, Maryland), BTX Molecular Delivery Systems (Holliston, MA) and Copernicus Therapeutics Inc, (see for example U.S. Pat. No. 6,008,336). Lipofection is described in e.g., U.S. Pat. Nos. 5,049,386; 4,946,787; and 4,897,355) and lipofection reagents are sold commercially (e.g., Transfectam™, Lipofectin™, and Lipofectamine™ RNAiMAX). Cationic and neutral lipids that are suitable for efficient receptor-recognition lipofection of polynucleotides include those of Felgner, International Patent Publication Nos. WO 91/17424 and WO 91/16024. Delivery can be to cells (ex vivo administration) or target tissues (in vivo administration).

The preparation of lipid:nucleic acid complexes, including targeted liposomes such as immunolipid complexes, is well known to one of skill in the art (see, e.g., Crystal (1995) Science 270:404-410; Blaese, et al. (1995) Cancer Gene Ther. 2:291-297; Behr, et al. (1994) Bioconjugate Chem. 5:382-389; Remy, et al. (1994) Bioconjugate Chem. 5:647-654; Gao, et al. (1995) Gene Therapy 2:710-722; Ahmad, et al. (1992) Cancer Res. 52:4817-4820; U.S. Pat. Nos. 4,186,183; 4,217,344; 4,235,871; 4,261,975; 4,485,054; 4,501,728; 4,774,085; 4,837,028; and 4,946,787).

Additional methods of delivery include the use of packaging the nucleic acids to be delivered into EnGeneIC delivery vehicles (EDVs). These EDVs are specifically delivered to target tissues using bispecific antibodies where one arm of the antibody has specificity for the target tissue and the other has specificity for the EDV. The antibody brings the EDVs to the target cell surface and then the EDV is brought into the cell by endocytosis. Once in the cell, the contents are released (see MacDiarmid, et al. (2009) Nature Biotechnology 27(7):643).

The use of RNA or DNA viral based systems for the delivery of nucleic acids encoding engineered DNA-binding proteins, and/or donors (e.g., CARs or ACTRs) as desired takes advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of nucleic acids include, but are not limited to, retroviral, lentivirus, adenoviral, adeno-associated, vaccinia and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system depends on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immunodeficiency virus (SIV), human immunodeficiency virus (HIV), and combinations thereof (see, e.g., Buchscher, et al. (1992) J. Virol. 66:2731-2739; Johann, et al. (1992) J. Virol. 66:1635-1640; Sommerfelt, et al. (1990) Virol. 176:58-59; Wilson, et al. (1989) J. Virol. 63:2374-2378; Miller, et al. (1991) J. Virol. 65:2220-2224; International Patent Publication No. WO 1994/026877).

In applications in which transient expression is preferred, adenoviral based systems can be used. Adenoviral based vectors are capable of very high transduction efficiency in many cell types and do not require cell division. With such vectors, high titer and high levels of expression have been obtained. This vector can be produced in large quantities in a relatively simple system. Adeno-associated virus (“AAV”) vectors are also used to transduce cells with target nucleic acids, e.g., in the in vitro production of nucleic acids and peptides, and for in vivo and ex vivo gene therapy procedures (see, e.g., West, et al. (1987) Virology 160:38-47; U.S. Pat. No. 4,797,368; International Patent Publication No. WO 93/24641; Kotin (1994) Human Gene Therapy 5:793-801; Muzyczka (1994) J. Clin. Invest. 94:1351. Construction of recombinant AAV vectors are described in a number of publications, including U.S. Pat. No. 5,173,414; Tratschin, et al. (1985) Mol. Cell. Biol. 5:3251-3260; Tratschin, et al. (1984) Mol. Cell. Biol. 4:2072-2081; Hermonat & Muzyczka (1984) PNAS USA 81:6466-6470; and Samulski et al. (1989) J. Virol. 63:03822-3828.

At least six viral vector approaches are currently available for gene transfer in clinical trials, which utilize approaches that involve complementation of defective vectors by genes inserted into helper cell lines to generate the transducing agent.

pLASN and MFG-S are examples of retroviral vectors that have been used in clinical trials (Dunbar, et al. (1995) Blood 85:3048-305; Kohn, et al. (1995) Nat. Med. 1:1017-102; Malech, et al. (1997) PNAS USA 94:22 12133-12138). PA317/pLASN was the first therapeutic vector used in a gene therapy trial. (Blaese, et al. (1995) Science 270:475-480). Transduction efficiencies of 50% or greater have been observed for MFG-S packaged vectors. (Ellem, et al. (1997) Immunol Immunother. 44(1): 10-20; Dranoff, et al. (1997) Hum. Gene Ther. 1:111-2.

Recombinant adeno-associated virus vectors (rAAV) are a promising alternative gene delivery system based on the defective and nonpathogenic parvovirus adeno-associated type 2 virus. All vectors are derived from a plasmid that retains only the AAV 145 bp inverted terminal repeats flanking the transgene expression cassette. Efficient gene transfer and stable transgene delivery due to integration into the genomes of the transduced cell are key features for this vector system. (Wagner, et al. (1998) Lancet 351(9117): 1702-3, Kearns, et al. (1996) Gene Ther. 9:748-55). Other AAV serotypes, including AAV1, AAV3, AAV4, AAV5, AAV6, AAV8, AAV8.2, AAV9 and AAVrh10 and pseudotyped AAV such as AAV2/8, AAV⅖ and AAV2/6 can also be used in accordance with the present invention.

Replication-deficient recombinant adenoviral vectors (Ad) can be produced at high titer and readily infect a number of different cell types. Most adenovirus vectors are engineered such that a transgene replaces the Ad E1a, E1b, and/or E3 genes; subsequently the replication defective vector is propagated in human 293 cells that supply deleted gene function in trans. Ad vectors can transduce multiple types of tissues in vivo, including nondividing, differentiated cells such as those found in liver, kidney and muscle. Conventional Ad vectors have a large carrying capacity. An example of the use of an Ad vector in a clinical trial involved polynucleotide therapy for antitumor immunization with intramuscular injection (Sterman, et al. (1998) Hum. Gene Ther. 7:1083-9). Additional examples of the use of adenovirus vectors for gene transfer in clinical trials include Rosenecker, et al. (1996) Infection 24(1):5-10; Sterman, et al. (1998) Hum. Gene Ther. 9(7):1083-1089; Welsh, et al. (1995) Hum. Gene Ther. 2:205-18; Alvarez, et al. (1997) Hum. Gene Ther. 5:597-613; Topf, et al. (1998) Gene Ther. 5:507-513; Sterman, et al. (1998) Hum. Gene Ther. 7:1083-1089.

Packaging cells are used to form virus particles that are capable of infecting a host cell. Such cells include 293 cells, which package adenovirus, and _{Ψ}2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. In addition, AAV can be manufactured using a baculovirus system (see, e.g., U.S. Pat. Nos. 6,723,551 and 7,271,002).

Purification of AAV particles from a 293 or baculovirus system typically involves growth of the cells which produce the virus, followed by collection of the viral particles from the cell supernatant or lysing the cells and collecting the virus from the crude lysate. AAV is then purified by methods known in the art including ion exchange chromatography (e.g., see U.S. Pat. Nos. 7,419,817 and 6,989,264), ion exchange chromatography and CsCl density centrifugation (e.g., International Patent Publication No. WO 2011/094198 A10), immunoaffinity chromatography (e.g., International Patent Publication No. WO 2016/128408) or purification using AVB Sepharose (e.g., GE Healthcare Life Sciences).

In many gene therapy applications, it is desirable that the gene therapy vector be delivered with a high degree of specificity to a particular tissue type. Accordingly, a viral vector can be modified to have specificity for a given cell type by expressing a ligand as a fusion protein with a viral coat protein on the outer surface of the virus. The ligand is chosen to have affinity for a receptor known to be present on the cell type of interest. For example, Han, et al. (1995) Proc. Natl. Acad. Sci. USA 92:9747-9751, reported that Moloney murine leukemia virus can be modified to express human heregulin fused to gp70, and the recombinant virus infects certain human breast cancer cells expressing human epidermal growth factor receptor. This principle can be extended to other virus-target cell pairs, in which the target cell expresses a receptor and the virus expresses a fusion protein comprising a ligand for the cell-surface receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences which favor uptake by specific target cells.

Gene therapy vectors can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by re-implantation of the cells into a patient, usually after selection for cells which have incorporated the vector.

The cells described herein may also be used for cell therapies, for example adoptive cell therapy for treatment and/or prevention of a cancer. Cell therapy is a specialized type of transplant wherein cells of a certain type (e.g., T cells reactive to a tumor antigen or B cells) are given to a recipient. Cell therapy can be done with cells that are either autologous (derived from the recipient) or allogenic (derived from a donor) and the cells may be immature cells such as stem cells, or completely mature and functional cells such as T cells. In fact, in some diseases such certain cancers, T cells may be manipulated ex vivo to increase their avidity for certain tumor antigens, expanded and then introduced into the patient suffering from that cancer type in an attempt to eradicate the tumor. This is particularly useful when the endogenous T cell response is suppressed by the tumor itself.

Ex vivo cell transfection for diagnostics, research, transplant or for gene and/or cell therapy (e.g., via re-infusion of the transfected cells into the host organism) is well known to those of skill in the art. In a preferred embodiment, cells are isolated from the subject organism, transfected with a DNA-binding proteins nucleic acid (gene or cDNA), and re-infused back into the subject organism (e.g., patient). Various cell types suitable for ex vivo transfection are well known to those of skill in the art (see, e.g., Freshney, et al., Culture of Animal Cells, A Manual of Basic Technique (3rd ed. 1994)) and the references cited therein for a discussion of how to isolate and culture cells from patients).

In one embodiment, stem cells are used in ex vivo procedures for cell transfection and gene therapy. The advantage to using stem cells is that they can be differentiated into other cell types in vitro or can be introduced into a mammal (such as the donor of the cells) where they will engraft in the bone marrow. Methods for differentiating CD34+ cells in vitro into clinically important immune cell types using cytokines such a GM-CSF, IFN-γ and TNF-α are known (see Inaba, et al. (1992) J. Exp. Med. 176:1693-1702).

Stem cells are isolated for transduction and differentiation using known methods. For example, stem cells are isolated from bone marrow cells by panning the bone marrow cells with antibodies which bind unwanted cells, such as CD4+ and CD8+ (T cells), CD45+ (panB cells), GR-1 (granulocytes), and Iad (differentiated antigen presenting cells) (see Inaba, et al. (1992) J. Exp. Med. 176: 1693-1702).

Stem cells that have been modified may also be used in some embodiments. For example, neuronal stem cells that have been made resistant to apoptosis may be used as therapeutic compositions where the stem cells also contain the ZFP TFs of the invention. Resistance to apoptosis may come about, for example, by knocking out BAX and/or BAK using BAX- or BAK-specific ZFNs (see, U.S. Pat. No. 8,597,912) in the stem cells, or those that are disrupted in a caspase, again using caspase-6 specific ZFNs for example. These cells can be transfected with the ZFP TFs that are known to regulate TCR.

Vectors (e.g., retroviruses, adenoviruses, liposomes, etc.) containing therapeutic DNA-binding proteins (or nucleic acids encoding these proteins) can also be administered directly to an organism for transduction of cells in vivo. Alternatively, naked DNA can be administered. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Methods for introduction of DNA into hematopoietic stem cells are disclosed, for example, in U.S. Pat. No. 5,928,638. Vectors useful for introduction of transgenes into hematopoietic stem cells, e.g., CD34+ cells, include adenovirus Type 35.

Vectors suitable for introduction of transgenes into immune cells (e.g., T-cells) include non-integrating lentivirus vectors. See, for example, Ory, et al. (1996) Proc. Natl. Acad. Sci. USA 93:11382-11388; Dull, et al. (1998)J. Virol. 72:8463-8471; Zuffery, et al. (1998) J. Virol. 72:9873-9880; Follenzi, et al. (2000) Nature Genetics 25:217-222.

Pharmaceutically acceptable carriers are determined in part by the particular composition being administered, as well as by the particular method used to administer the composition. Accordingly, there is a wide variety of suitable formulations of pharmaceutical compositions available, as described below (see, e.g., Remington’s Pharmaceutical Sciences, 17th ed., 1989).

As noted above, the disclosed methods and compositions can be used in any type of cell including, but not limited to, prokaryotic cells, fungal cells, Archaeal cells, plant cells, insect cells, animal cells, vertebrate cells, mammalian cells and human cells, including T-cells and stem cells of any type. Suitable cell lines for protein expression are known to those of skill in the art and include, but are not limited to COS, CHO (e.g., CHO-S, CHO-K1, CHO-DG44, CHO-DUXB11), VERO, MDCK, WI38, V79, B14AF28-G3, BHK, HaK, NS0, SP2/0-Agl4, HeLa, HEK293 (e.g., HEK293-F, HEK293-H, HEK293-T), perC6, insect cells such as Spodoptera fugiperda (Sf), and fungal cells such as Saccharomyces, Pichia and Schizosaccharomyces. Progeny, variants and derivatives of these cell lines can also be used.

### Applications

The disclosed compositions and methods can be used for any application in which it is desired to modulate TCR and/or B2M expression and/or functionality, including but not limited to, therapeutic and research applications in which TCR and/or B2M modulation is desirable. For example, the disclosed compositions can be used in vivo and/or ex vivo (cell therapies) to disrupt the expression of endogenous TCRs and/or B2M in T cells modified for adoptive cell therapy to express one or more exogenous CARs, exogenous TCRs, or other cancer-specific receptor molecules, thereby treating and/or preventing the cancer. T cells may be effector T cells or regulatory T cells. In addition, in such settings, abrogation of TCR expression within a cell can eliminate or substantially reduce the risk of an unwanted cross reaction with healthy, nontargeted tissue (i.e. a graft-vs-host response). Modified cells as described herein can also be used for treatment of cancers, including, but not limited to, prostate, chronic lymphocytic leukemia (CLL) and Non-Hodgkin’s lymphomas.

Methods and compositions also include stem cell compositions (e.g., iPSC and HSC/HSPC) wherein the B2M, TCRA and/or TCRB genes within the stem cells has been modulated (modified) and the cells further comprise an ACTR and/or a CAR and/or an isolated or engineered TCR. For example, TCR knock out or knock down modulated allogeneic hematopoietic stem cells can be introduced into an HLA-matched patient following bone marrow ablation. These altered HSC would allow the re-colonization of the patient but would not cause potential GvHD. The introduced cells may also have other alterations to help during subsequent therapy (e.g., chemotherapy resistance) to treat the underlying disease. The HLA class I null cells also have use as an “off the shelf” therapy in emergency room situations with trauma patients.

The methods and compositions of the invention are also useful for the design and implementation of in vitro and in vivo models, for example, animal models of TCR or B2M and associated disorders, which allows for the study of these disorders.

All patents, patent applications and publications mentioned herein are hereby incorporated by reference in their entireties.

Although disclosure has been provided in some detail by way of illustration and example for the purposes of clarity and understanding, it will be apparent to those of skill in the art that various changes and modifications can be practiced without departing from the spirit or scope of the disclosure. Accordingly, the foregoing disclosure and following examples should not be construed as limiting.

### EXAMPLES

### Example 1: Design of TCR-Specific Nucleases

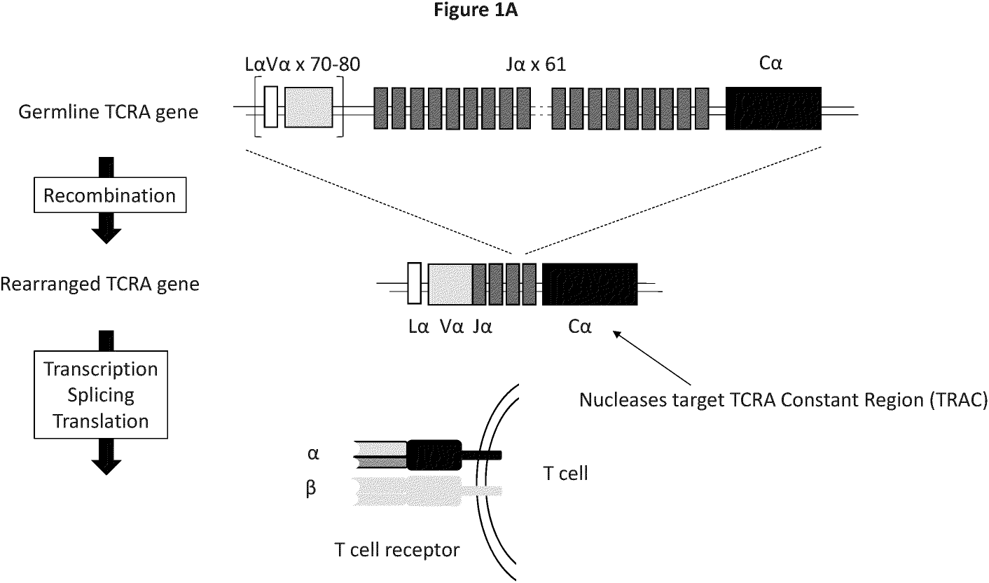

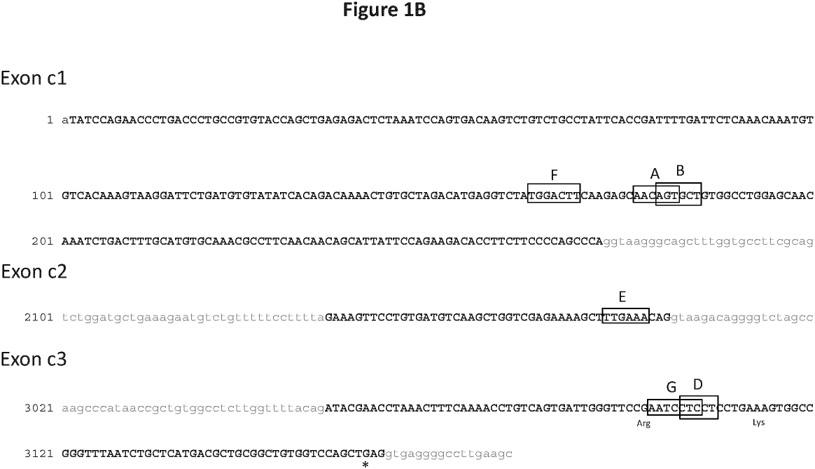

TCR-specific ZFNs were constructed to enable site specific introduction of double strand breaks at the TCRα (TCRA) gene. ZFNs were designed essentially as described in Urnov, et al. (2005) Nature 435(7042):646-651, Lombardo, et al. (2007) Nat Biotechnol. 25(11):1298-306, and U.S. Pat. Publication Nos. 2008/0131962; 2015/0164954; 2014/0120622; and 2014/0301990 and U.S. Pat. No. 8,956,828. The ZFN pairs targeted different sites in the constant region of the TCRA gene (see FIG. 1). The recognition helices for exemplary ZFN pairs as well as the target sequence are shown below in Table 1. Target sites of the TCRA zinc-finger designs are shown in the first column. Nucleotides in the target site that are targeted by the ZFP recognition helices are indicated in uppercase letters; non-targeted nucleotides indicated in lowercase. Linkers used to join the FokI nuclease domain and the ZFP DNA binding domain are also shown (see U.S. Pat. Publication No. 2015/0132269). For example, the amino acid sequence of the domain linker L0 is DNA binding domain-QLVKS-FokI nuclease domain (SEQ ID NO:5). Similarly, the amino acid sequences for the domain linker N7a is FokI nuclease domain-SGTPHEVGVYTL-DNA binding domain (SEQ ID NO:6), and N7c is FokI nuclease domain-SGAIRCHDEFWF-DNA binding domain (SEQ ID NO:7).

TABLE 1

TCR-α (TCRA) Zinc-finger Designs

| | | | | | | | |
|---|---|---|---|---|---|---|---|
| ZFN Name target sequence | F1 | F2 | F3 | F4 | F5 | F6 | Domain linker |
| SBS55204 5′ttGCTC TTGAAGTC cATAGACc tcatgt (SEQ ID NO:8) | DRSNLSR (SEQ ID NO:22) | QKVTLAA (SEQ ID NO:23) | DRSALSR (SEQ ID NO:24) | TSGNLTR (SEQ ID NO:25) | YRSSLKE (SEQ ID NO:26) | TSGNLTR (SEQ ID NO:25) | L0 |
| SBS53759 5′gtGCTG TGgCCTGG AGCAACAa atctga (SEQ ID NO:9) | QQNVLIN (SEQ ID NO:27) | QNATRTK (SEQ ID NO:28) | QSGHLAR (SEQ ID NO:29) | NRYDLMT (SEQ ID NO:30) | RSDSLLR (SEQ ID NO:31) | QSSDLTR (SEQ ID NO:32) | L0 |
| SBS55229 5′ctGTTG CTCTTGAA GTCcatag acctca (SEQ ID NO:10) | DRSALAR (SEQ ID NO:33) | QSGNLAR (SEQ ID NO:34) | HRSTLQG (SEQ ID NO:35) | QSGDLTR (SEQ ID NO:36) | TSGSLTR (SEQ ID NO:37) | NA | La |
| SBS53785 5′ctGTGG CCtGGAGC AACAaatc tgactt | QHQVLVR (SEQ ID NO:38) | QNATRTK (SEQ ID NO:28) | QSGHLSR (SEQ ID NO:39) | DRSDLSR (SEQ ID NO:40) | RSDALAR (SEQ ID NO:41) | NA | L0 |
| (SEQ ID NO:11) | | | | | | | |
| SBS53810 5′agGATT CGGAACCC AATCACtg (SEQ ID NO:12) | DQSNLRA (SEQ ID NO:42) | TSSNRKT (SEQ ID NO:43) | DSSTRKT (SEQ ID NO:44) | QSGNLAR (SEQ ID NO:34) | RSDDLSE (SEQ ID NO:45) | TNSNRKR (SEQ ID NO:46) | L0 |
| SBS55255 5′ctCCTG AAAGTGGC CGGgttta atctgc (SEQ ID NO:13) | RSDHLST (SEQ ID NO:47) | DRSHLAR (SEQ ID NO:48) | LKQHLNE (SEQ ID NO:49) | TSGNLTR (SEQ ID NO:25) | HRTSLTD (SEQ ID NO:50) | NA | L0 |
| SBS55248 5′agGATT CGGAACCC AATCACtg acaggt (SEQ ID NO:14) | DQSNLRA (SEQ ID NO:42) | TSSNRKT (SEQ ID NO:43) | LQQTLAD (SEQ ID NO:51) | QSGNLAR (SEQ ID NO:34) | RREDLIT (SEQ ID NO:52) | TSSNLSR (SEQ ID NO:53) | L0 |
| SBS55254 5′ctCCTG AAAGTGGC CGGgttta atctgc (SEQ ID NO:13) | RSDHLST (SEQ ID NO:47) | DRSHLAR (SEQ ID NO:48) | LKQHLNE (SEQ ID NO:49) | QSGNLAR (SEQ ID NO:34) | HNSSLKD (SEQ ID NO:54) | NA | L0 |
| SBS55260 5′ctCCTG AAAGTGGC CGGgttta atctgc (SEQ ID NO:13) | RSDHLST (SEQ ID NO:47) | DRSHLAR (SEQ ID NO:48) | LNHHLQQ (SEQ ID NO:55) | QSGNLAR (SEQ ID NO:34) | HKTSLKD (SEQ ID NO:56) | NA | L0 |
| SBS55266 5′tcAAGC TGGTCGAG aAAAGCTt tgaaac (SEQ ID NO:15) | QSSDLSR (SEQ ID NO:57) | QSGNRTT (SEQ ID NO:58) | RSANLAR (SEQ ID NO:59) | DRSALAR (SEQ ID NO:33) | RSDVLSE (SEQ ID NO:60) | KHSTRRV (SEQ ID NO:61) | N7c |
| SBS53853 5′aaCAGG TAaGACAG GGGTCTAg cctggg (SEQ ID NO:16) | TMHQRVE (SEQ ID NO:62) | TSGHLSR (SEQ ID NO:63) | RSDHLTQ (SEQ ID NO:64) | DSANLSR (SEQ ID NO:65) | QSGSLTR (SEQ ID NO:66) | AKWNLDA (SEQ ID NO:67) | L0 |
| SBS53860 5′ctGTGC TAGACATG aGGTCTAt ggactt (SEQ ID | TMHQRVE (SEQ ID NO:62) | TSGHLSR (SEQ ID NO:63) | RNDSLKT (SEQ ID NO:68) | DSSNLSR (SEQ ID NO:69) | QKATRTT (SEQ ID NO: 70) | RNASRTR (SEQ ID NO:72) | N7a |
| NO:17) | | | | | | | |
| SBS53863 5′ttCAAG AGCAACAG tGCTGTGg cctgga (SEQ ID NO:18) | RSDSLLR (SEQ ID NO:31) | QSSDLRR (SEQ ID NO:73) | RSDNLSE (SEQ ID NO:74) | ERANRNS (SEQ ID NO:75) | RSDNLAR (SEQ ID NO:76) | QKVNLMS (SEQ ID NO:77) | L0 |
| SBS55287 5′ttCAAG AGCAACAG tGCTGTGg cctgga (SEQ ID NO:18) | RSDSLLR (SEQ ID NO:31) | QSSDLRR (SEQ ID NO:73) | RSDNLSE (SEQ ID NO:74) | ERANRNS (SEQ ID NO:75) | RSDNLAR (SEQ ID NO:76) | QKVNLRE (SEQ ID NO:78) | L0 |
| SBS53855 5′ctGTGC TAGACATG aGGTCTAt ggactt (SEQ ID NO:17) | TMHQRVE (SEQ ID NO:62) | TSGHLSR (SEQ ID NO:63) | RSDTLSQ (SEQ ID NO:79) | DRSDLSR (SEQ ID NO:40) | QKATRTT (SEQ ID NO: 70) | RNASRTR (SEQ ID NO:72) | N7a |
| SBS53885 5′ccTGTC AGtGATTG GGTTCCGa atcctc (SEQ ID NO:19) | RSDTLSE (SEQ ID NO:186) | TSGSLTR (SEQ ID NO:37) | RSDHLST (SEQ ID NO:47) | TSSNRTK (SEQ ID NO:71) | RSDNLSE (SEQ ID NO:74) | WHSSLRV (SEQ ID NO:83) | N7a |
| SBS52774 5′ccTGTC AGtGATTG GGTTCCGa atcctc (SEQ ID NO:19) | RKQTRTT (SEQ ID NO:80) | HRSSLRR (SEQ ID NO:81) | RSDHLST (SEQ ID NO:47) | TSANLSR (SEQ ID NO:82) | RSDNLSE (SEQ ID NO:74) | WHSSLRV (SEQ ID NO:83) | N7a |
| SBS53909 5′tcCTCC TGAAAGTG GCCGGGtt taatct (SEQ ID NO:20) | RSAHLSR (SEQ ID NO:84) | DRSDLSR (SEQ ID NO:40) | RSDVLSV (SEQ ID NO:85) | QNNHRIT (SEQ ID NO:86) | RSDVLSE (SEQ ID NO:60) | SPSSRRT (SEQ ID NO:87) | L0 |
| SBS52742 5′tcCTCC TGAAAGTG GCCGGGtt taatct (SEQ ID NO:20) | RSAHLSR (SEQ ID NO:84) | DRSDLSR (SEQ ID NO:40) | RSDSLSV (SEQ ID NO:88) | QNANRKT (SEQ ID NO: 89) | RSDVLSE (SEQ ID NO:60) | SPSSRRT (SEQ ID NO:87) | L0 |
| SBS53856 5′ctGTGC TAGACATG aGGTCTAt g (SEQ ID | TMHQRVE (SEQ ID NO:62) | TSGHLSR (SEQ ID NO:63) | RSDSLST (SEQ ID NO:90) | DRANRIK (SEQ ID NO:91) | QKATRTT (SEQ ID NO: 70) | RNASRTR (SEQ ID NO:72) | N7a |
| NO:21) | | | | | | | - |

All ZFNs were tested and found to bind to their target sites and found to be active as nucleases.

The ZFPs as described herein may also include one or more mutations to phosphate contact residues of the zinc finger protein and/or the FokI domain, for example, the nR-5Qabc mutant (to ZFP backbone) and/or R416S and/or K525S mutants (to FokI), described in U.S. Pat. Publication No. 20180087072.

Guide RNAs for the S. pyogenes CRISPR/Cas9 system were also constructed to target the TCRA gene. See, also, U.S. Pat. Publication No. 2015/0056705 for additional TCR alpha-targeted guide RNAs. The target sequences in the TCRA gene are indicated as well as the guide RNA sequences in Table 2 below. All guide RNAs are tested in the CRISPR/Cas9 system and are found to be active.

TABLE 2

Guide RNAs for the constant region of human TCRA (TRAC)

| | | | |
|---|---|---|---|
| Name | Strand | Target (5′->3′) | gRNA (5′ -> 3′) |
| TRAC-Gr14 | R | GCTGGTACACGGCAGGGTCAGGG (SEQ ID NO:92) | GCTGGTACACGGCAGGGTCA (SEQ ID NO:104) |
| TRAC-Gr25 | R | AGAGTCTCTCAGCTGGTACACGG (SEQ ID NO:93) | gAGAGTCTCTCAGCTGGTACA (SEQ ID NO:105) |
| TRAC-Gr71 | R | GAGAATCAAAATCGGTGAATAGG (SEQ ID NO:94) | GAGAATCAAAATCGGTGAAT (SEQ ID NO:106) |
| TRAC-Gf155 | F | ACAAAACTGTGCTAGACATGAGG (SEQ ID NO:95) | gACAAAACTGTGCTAGACATG (SEQ ID NO:107) |
| TRAC-Gf191 | F | AGAGCAACAGTGCTGTGGCCTGG (SEQ ID NO:96) | gAGAGCAACAGTGCTGTGGCC (SEQ ID NO:108) |
| TRAC-Gf271 | F | GACACCTTCTTCCCCAGCCCAGG (SEQ ID NO:97) | GACACCTTCTTCCCCAGCCC (SEQ ID NO:109) |
| TRAC-Gr2146 | R | CTCGACCAGCTTGACATCACAGG (SEQ ID NO:98) | gCTCGACCAGCTTGACATCAC (SEQ ID NO:110) |
| TRAC-Gf2157 | F | AAGTTCCTGTGATGTCAAGCTGG (SEQ ID NO:99) | gAAGTTCCTGTGATGTCAAGC (SEQ ID NO:111) |
| TRAC-Gf2179 | F | GTCGAGAAAAGCTTTGAAACAGG (SEQ ID NO:100) | GTCGAGAAAAGCTTTGAAAC (SEQ ID NO:112) |
| TRAC-Gr3081 | R | TTCGGAACCCAATCACTGACAGG (SEQ ID NO:101) | gTTCGGAACCCAATCACTGAC (SEQ ID NO:113) |
| TRAC-Gr3099 | R | CCACTTTCAGGAGGAGGATTCGG (SEQ ID NO:102) | gCCACTTTCAGGAGGAGGATT (SEQ ID NO:114) |
| TRAC-Gr3105 | R | ACCCGGCCACTTTCAGGAGGAGG (SEQ ID NO:103) | gACCCGGCCACTTTCAGGAGG (SEQ ID NO:115) |

Thus, the nucleases described herein (e.g., nucleases comprising a ZFP or a sgRNA DNA-binding domain) bind to their target sites and cleave the TCRA gene, thereby making genetic modifications within a TCRA gene comprising any of SEQ ID NO:6-48 or 137-205, including modifications (insertions and/or deletions) within any of these sequences (e.g., the target sequences shown in any of SEQ ID NO:8-21 and/or 92-103; 12-25 nucleotides of these target sites; and/or between paired target sites) and/or modifications within the following sequences: AACAGT, AGTGCT, CTCCT, TTGAAA, TGGACTT and/or AATCCTC (see, FIG. 1B). TALE nucleases targeted to these target sites are also designed and found to be functional in terms of binding and activity.

Furthermore, the DNA-binding domains (ZFPs and sgRNAs) all bound to their target sites and ZFP, TALE and sRNA DNA-binding domains that recognize these target sites are also formulated into active engineered transcription factors when associated with one or more transcriptional regulatory domains.

### Example 2: Nuclease Activity in Vitro

The ZFNs described in Table 1 were used to test nuclease activity in K562 cells. To test cleavage activity, plasmids encoding the pairs of human TCRA-specific ZFNs described above were transfected into K562 cells with plasmid or mRNAs. K562 cells were obtained from the American Type Culture Collection and grown as recommended in RPMI medium (Invitrogen) supplemented with 10% qualified fetal bovine serum (FBS, Cyclone). For transfection, ORFs for the active nucleases listed in Table 1 were cloned into an expression vector optimized for mRNA production bearing a 5′ and 3′ UTRs and a synthetic polyA signal. The mRNAs were generated using the mMessage mMachine T7 Ultra kit (Ambion) following the manufacturer’s instructions. In vitro synthesis of nuclease mRNAs used either a pVAX-based vector containing a T7 promoter, the nuclease proper and a polyA motif for enzymatic addition of a polyA tail following the in vitro transcription reaction, or a pGEM based vector containing a T7 promoter, a 5′UTR, the nuclease proper, a 3′UTR and a 64 bp polyA stretch (SEQ ID NO: 188), or a PCR amplicon containing a T7 promoter, a 5′UTR, the nuclease proper, a 3′UTR and a 60 bp polyA stretch (SEQ ID NO: 189). One million K562 cells were mixed with 250 ng or 500 ng of the ZFN encoding mRNA. Cells were transfected in an Amaxa Nucleofector IITM using program T-16 and recovered into 1.4 mL warm RPMI medium + 10% FBS. Nuclease activity was assessed by deep sequencing (MiSeq, Illumina) as per standard protocols three days following transfection. The results are presented below in Table 3.

TABLE 3

Zinc Finger Nuclease activity

| Pair # | ZFN pair | NHEJ% (250 ng/ZFN) | SD | NHEJ% (500 ng/ZFN) | SD | Site |
|---|---|---|---|---|---|---|
| 1 | 55204:53759 | 76.7 | 1.3 | 87.7 | 1 | A2 |
| 2 | 55229:53785 | 91.4 | 1.5 | 93.6 | 1.7 | B |
| 3 | 53810:55255 | 81.6 | 0.6 | 91.5 | 1.3 | D1 |
| 4 | 55248:55254 | 95.4 | 1.8 | 96.2 | 1.2 | D2 |
| 5 | 55248:55260 | 87.9 | 1.3 | 93.0 | 1 | D3 |
| 6 | 55266:53853 | 85.3 | 1.4 | 88.9 | 0.4 | E |
| 7 | 53860:53863 | 77.1 | 1.7 | 87.3 | 1.1 | F1 |
| 8 | 53856:55287 | 53.6 | 3.2 | 74.8 | 1.3 | F2 |
| 9 | 53885:53909 | 90.1 | 1.6 | 90.2 | 1.5 | G1 |
| 10 | 52774:52742 | 76.8 | 0.8 | 84.4 | 2.2 | GO |
| 11 | GFP | 0 | | 0 | | |

Highly active TCRA specific TALENs have also been previously described (see International Patent Publication No. WO 2014/153470).

The human TCRA-specific CRISPR/Cas9 systems were also tested. The activity of the CRISPR/Cas9 systems in human K562 cells was measured by MiSeq analysis. Cleavage of the endogenous TCRA DNA sequence by Cas9 is assayed by high-throughput sequencing (Miseq, Illumina).

In these experiments, Cas9 was supplied on a pVAX plasmid, and the sgRNA is supplied on a plasmid under the control of a promoter (e.g., the U6 promoter or a CMV promoter). The plasmids were mixed at either 100 ng of each or 400 ng of each and were mixed with 2e5 cells per run. The cells were transfected using the Amaxa system. Briefly, an Amaxa transfection kit is used and the nucleic acids are transfected using a standard Amaxa shuttle protocol. Following transfection, the cells are let to rest for 10 minutes at room temperature and then resuspended in prewarmed RPMI. The cells are then grown in standard conditions at 37° C. Genomic DNA was isolated 7 days after transfection and subject to MiSeq analysis.

Briefly, the guide RNAs listed in Table 2 were tested for activity. The guide RNAs were tested in three different configurations: G0 is the set up described above. G1 used a pVAX vector comprising a CMV promoter driving expression of the Cas9 gene and a U6-Guide RNA-tracer expression cassette where transcription of both reading frames is in the same orientation. G2 is similar to G1 except that the Cas9 and U6-Guide expression cassettes are in opposite orientations. These three set ups were tested using either 100 ng or 400 ng of transfected DNA, and the results are presented below in Table 4. Results are expressed as the ‘percent indels’ or “NHEJ%’, where ‘indels’ means small insertions and/or deletions found as a result of the error prone NHEJ repair process at the site of a nuclease-induced double strand cleavage.

TABLE 4

CRISPR/Cas activity
% total_indels

| | GR0 | | GR1 | | GR2 | |
|---|---|---|---|---|---|---|
| Guide used | NHEJ% (100 ng) | NHEJ% (400 ng) | NHEJ% (100 ng) | NHEJ% (400 ng) | NHEJ% (100 ng) | NHEJ% (400 ng) |
| TCRA-Gr14 | 6.4 | 25.8 | 0.6 | 12.4 | 0.5 | 10.2 |
| TCRA-Gr25 | 14.6 | 26.9 | 2.4 | 21.7 | 1.1 | 21.6 |
| TCRA-Gr71 | 3.7 | 13.8 | 0.3 | 4.2 | 0.3 | 7.8 |
| TCRA-Gf155 | 6.0 | 19.5 | 1.2 | 12.7 | 0.8 | 15.9 |
| TCRA-Gf191 | 1.0 | 6.9 | 0.3 | 2.3 | 0.4 | 4.5 |
| TCRA-Gf271 | 4.7 | 21.5 | 0.8 | 10.3 | 0.7 | 15.2 |
| TCRA-Gr2146 | 1.1 | 8.8 | 0.3 | 1.7 | 0.2 | 2.0 |
| TCRA-Gf2157 | 3.8 | 22.2 | 0.6 | 9.6 | 0.6 | 12.0 |
| TCRA-Gf2179 | 0.8 | 4.9 | 0.2 | 1.8 | 0.2 | 1.4 |
| TCRA-Gr3081 | 5.9 | 23.6 | 0.7 | 11.5 | 0.8 | 12.6 |
| TCRA-Gr3099 | 2.1 | 21.1 | 0.4 | 7.1 | 0.3 | 6.2 |
| TCRA-Gr3105 | 12.1 | 45.9 | 2.2 | 22.0 | 1.0 | 7.6 |
| ZFN controls | | | | | | |
| 55248:55254 | 24.2 | 52.4 | | | | |
| 55229:53785 | 6.0 | 24.5 | | | | |
| 55266:53853 | 12.0 | 37.0 | | | | |

As shown, the nucleases described herein induce cleavage and genomic modifications at the targeted site.

Thus, the nucleases described herein (e.g., nucleases comprising a ZFP, a TALE or a sgRNA DNA-binding domain) bind to their target sites and cleave the TCRA gene, thereby making genetic modifications within a TCRA gene comprising any of SEQ ID NO:8-21 or 92-103, including modifications (insertions and/or deletions) within any of these sequences (SEQ ID NO:8-21, 92-103); modifications within 1-50 (e.g., 1 to 10) base pairs of these gene sequences; modifications between target sites of paired target sites (for dimers); and/or modifications within one or more of the following sequences: AACAGT, AGTGCT, CTCCT, TTGAAA, TGGACTT and/or AATCCTC (see, FIG. 1B).

Furthermore, the DNA-binding domains (ZFPs, TALEs and sgRNAs) all bound to their target sites and are also formulated into active engineered transcription factors when associated with one or more transcriptional regulatory domains.

### Example 3: TCRA-Specific ZFN Activity in T Cells

The TCRA-specific ZFN pairs were also tested in human T cells for nuclease activity. mRNAs encoding the ZFNs were transfected into purified T cells. Briefly, T cells were obtained from leukopheresis product and purified using the Miltenyi CliniMACS system (CD4 and CD8 dual selection). These cells were then activated using Dynabeads (ThermoFisher) according to manufacturer’s protocol. 3 days post activation, the cells were transfected with three doses of mRNA (60, 120 and 250 µg/mL) using a Maxcyte electroporator (Maxcyte), OC-100, 30e6 cells/mL, volume of 0.1 mL. Cells were analyzed for on target TCRA modification using deep sequencing (Miseq, Illumina) at 10 days after transfection. Cell viability and cell growth (total cell doublings) were measured throughout the 13-14 days of culture. In addition, TCR on the cell surface of the treated cells was measured using standard FACS analysis at day 10 of culture staining for CD3.

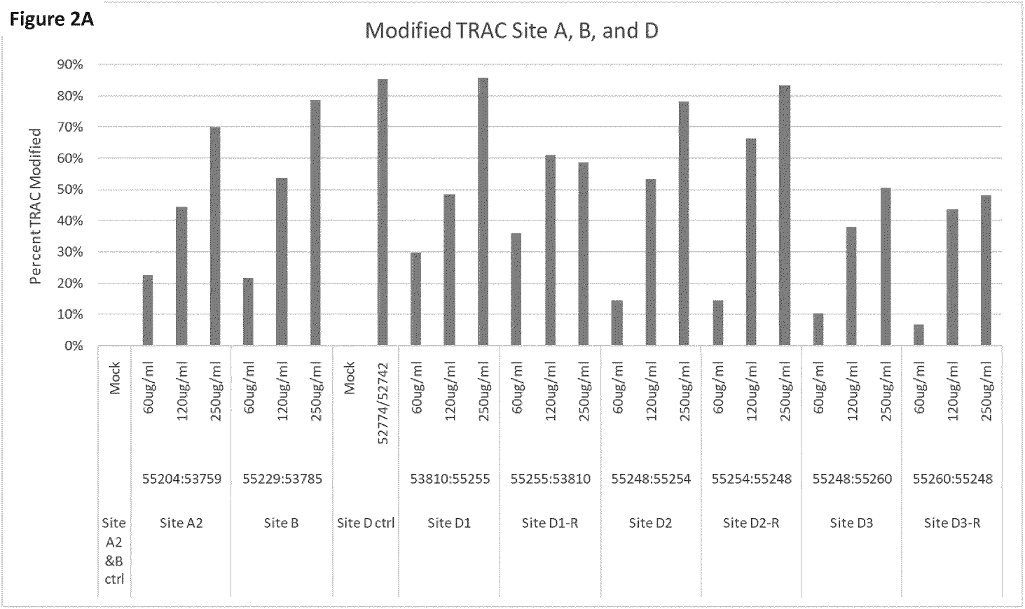

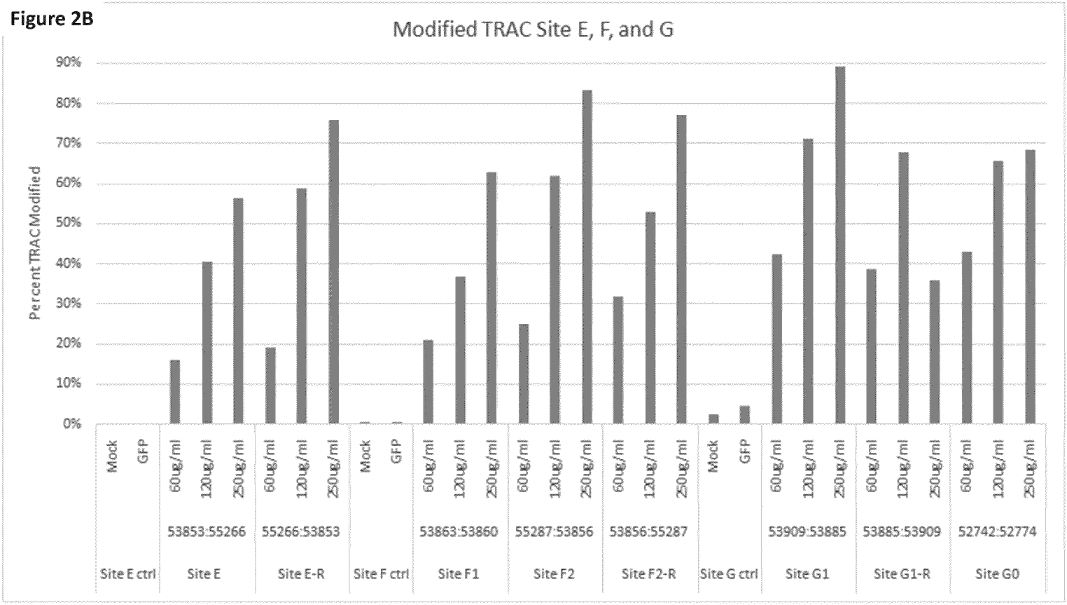

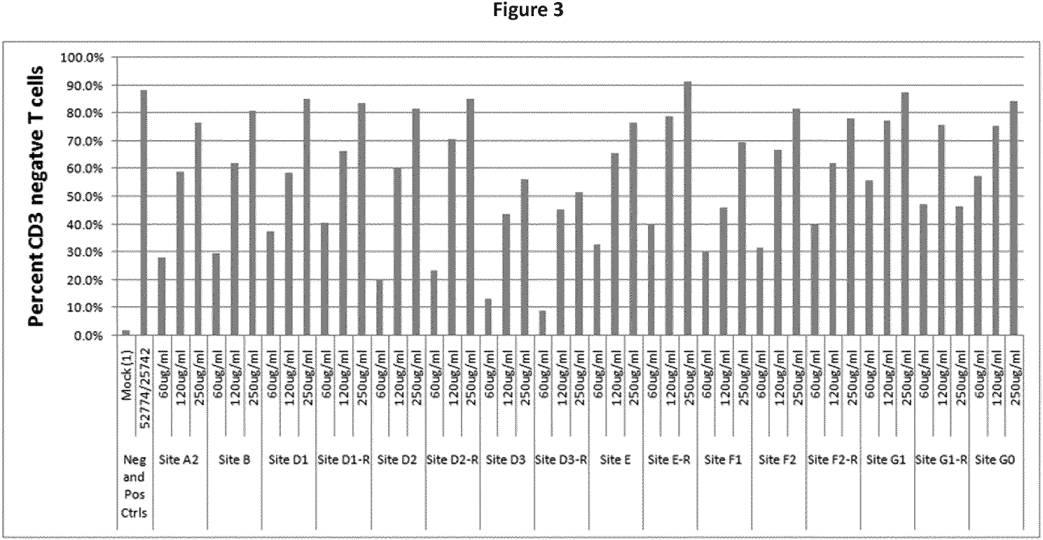

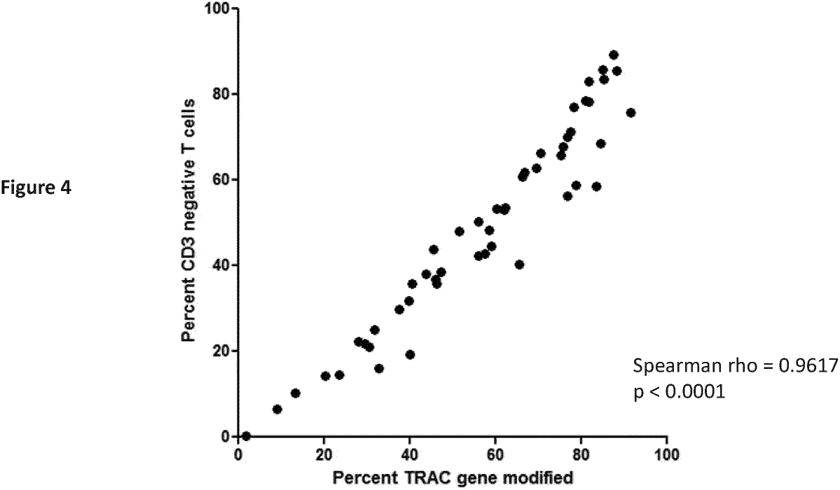

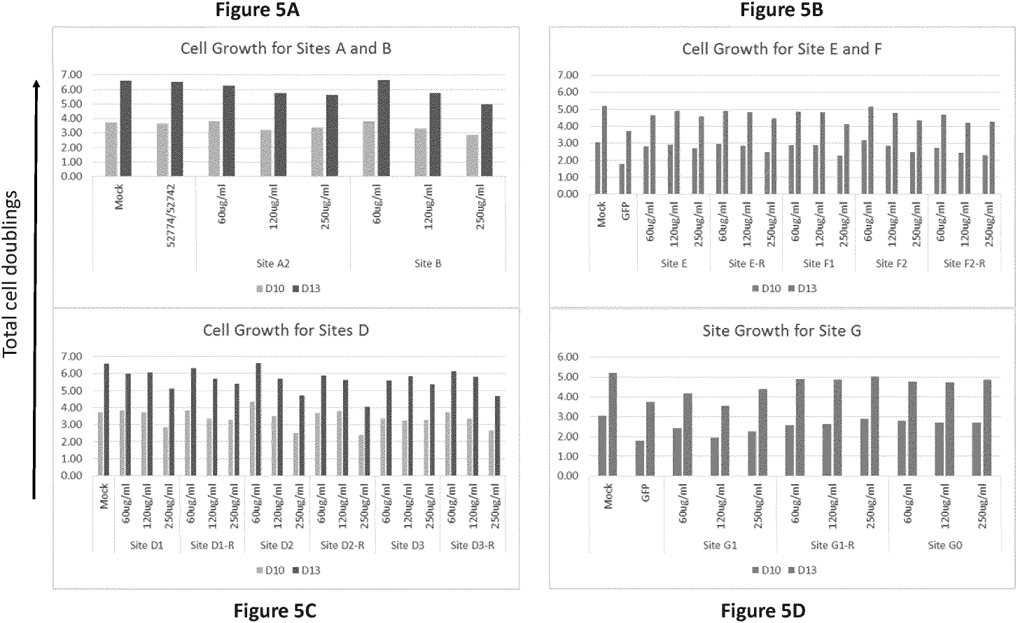

The TCRA-specific ZFN pairs were all active in T cells and some were capable of causing more than 80% TCRA allele modification in these conditions (see FIGS. 2A and 2B). Similarly, T cells treated with the ZFNs lost expression of CD3, where FACS analysis showed that in some cases between 80 and 90% of the T cells were CD3 negative (FIG. 3). A comparison between percent TCRA modified by ZFN and CD3 loss in these cells demonstrated a high degree of correlation (FIG. 4). Cell viability was comparable to the mock treatment controls, and TCRA knockout cell growth was also comparable to the controls (see FIGS. 5A-5D).

### Example 4: Double Knockout of B2M and TCRA With Targeted Integration

Nucleases as described above and B2M targeted nuclease described in Table 5 (see, also U.S. Pat. Publication No. 2017/0173080) were used to inactivate B2M and TCRA and to introduce, via targeted integration, a donor (transgene) into either the TCRA or B2M locus. The B2M specific ZFNs are shown below in Table 5:

TABLE 5

B2M-specific ZFN designs

| | | | | | | | |
|---|---|---|---|---|---|---|---|
| ZFN Name target sequence | F1 | F2 | F3 | F4 | F5 | F6 | Domain linker |
| SBS57327 5′ taGCAATTC AGGAAaTTT GACtttcca t (SEQ ID NO:123) | DRSNLSR (SEQ ID NO:22) | ARWYLDK (SEQ ID NO:125) | QSGNLAR (SEQ ID NO:34) | AKWNLDA (SEQ ID NO : 67) | QQHVLQN (SEQ ID NO:119) | QNATRTK (SEQ ID NO: 28) | L0 |
| SBS57332 5′tgTCGGA TgGATGAAA CCCAGacac ata (SEQ ID NO:117) | RSDNLSE (SEQ ID NO:74) | ASKTRTN (SEQ ID NO:120) | QSGNLAR (SEQ ID NO:34) | TSANLSR (SEQ ID NO: 82) | TSGNLTR (SEQ ID NO:25) | RTEDRLA (SEQ ID NO:121) | N6a |
| SBS57531 5′ gaGTAGCGc GAGCACAGC taaggccac g (SEQ ID NO:126) | AQCCLFH (SEQ ID NO:128) | DQSNLRA (SEQ ID NO:42) | RSANLTR (SEQ ID NO:129) | RSDDLTR (SEQ ID NO:130) | QSGSLTR (SEQ ID NO: 66) | N/A | N6a |
| SBS57071 gcCACGGAg CGAGACATC TCGgcccga a (SEQ ID NO:127) | RSDDLSK (SEQ ID NO: 131) | DSSARKK (SEQ ID NO: 132) | DRSNLSR (SEQ ID NO:22) | QRTHLRD (SEQ ID NO:133) | QSGHLAR (SEQ ID NO:29) | DSSNREA (SEQ ID NO: 134) | L0 |

In this experiment, the TCRA-specific ZFN pair was SBS#55266/SBS#53853, comprising the sequence TTGAAA between the TCRA-specific ZFN target sites (Table 1), and the B2M pair was SBS#57332/SBS#57327 (Table 5), comprising the sequence TCAAAT between the B2M-specific ZFN target sites.

Briefly, T-Cells (AC-TC-006) were thawed and activated with CD3/28 dynabeads (1:3 cells:bead ratio) in X-vivo15 T-cell culture media (day 0). After two days in culture (day 2), an AAV donor (comprising a GFP transgene and homology arms to the TCRA or B2M gene) was added to the cell culture, except control groups without donor were also maintained. The following day (day 3), TCRA and B2M ZFNs were added via mRNA delivery in the following 5 Groups:
(a) Group 1 (TCRA and B2M ZFNs only, no donor): TCRA 120 ug/mL: B2M only 60ug/mL;
(b) Group 2 (TCRA and B2M ZFNs and donor with TCRA homology arms): TCRA 120 ug/mL; B2M 60 ug/mL and AAV (TCRA-Site E-hPGK-eGFP-Clone E2) 1E5vg/cell;
(c) Group 3 (TCRA and B2M ZFNs and donor with TCRA homology arms): TCRA 120 ug/mL; B2M 60 ug/mL; and AAV (TCRA-Site E-hPGK-eGFP-Clone E2) 3E4vg/cell;
(d) Group 4 (TCRA and B2M ZFNs and donor with B2M homology arms): TCRA 120 ug/mL; B2M 60 ug/mL and AAV (pAAV B2M -hPGK GFP) 1E5vg/cell
(e) Group 5 (TCRA and B2M ZFNs and donor with B2M homology arms): TCRA 120 ug/mL; B2M 60 ug/mL and AAV (pAAV B2M - hPGK GFP) 3E4vg/cell.
All experiments were conducted at 3e7cells/ml cell density using the protocol as described in U.S. Pat. Publication No. 2017/0137845 (extreme cold shock) and were cultured to cold shock at 30° C. overnight post electroporation.

The following day (day 4), cells were diluted to 0.5e6 cells/ml and transferred to cultures at 37° C. Three days later (day 7), cells diluted to 0.5e6 cells/ml again. After three and seven more days in culture (days 10 and 14, respectively), cells were harvested for FACS and MiSeq analysis (diluted to 0.5e6cells/ml).

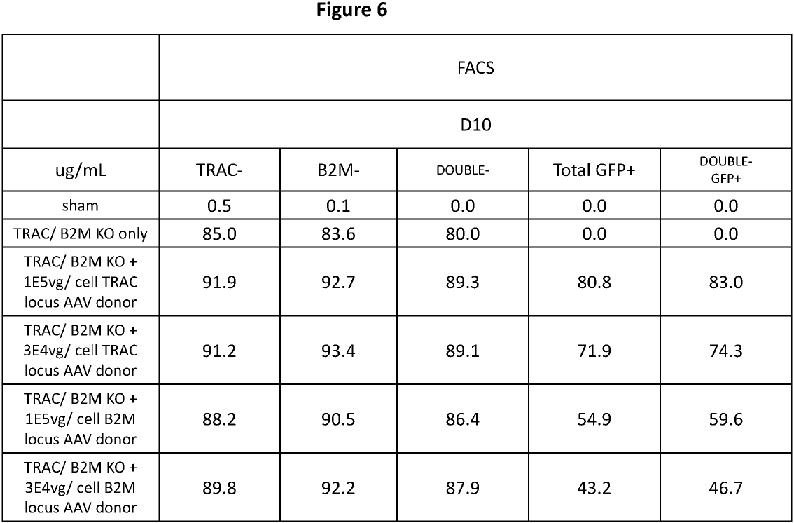

As shown in FIG. 6, GFP expression indicated that target integration was successful and that genetically modified cells comprising B2M and TCRA modifications (insertions and/or deletions) within the nuclease target sites (or within 1 to 50, 1-20, 1-10 or 1-5 base pairs of the nuclease target sites), including within the TTGAAA and TCAAAT (between the paired target sites) as disclosed herein were obtained.

Additional experiments were performed to generate cells with double-knockouts of TRAC and B2M and targeted integration of a donor vector. In particular, the TRAC-specific ZFN pair SBS#55266/SBS#53853 and the B2M pair SBS#57071/SBS#57531 were introduced into T-cells. Briefly, a 1:1 ratio of CD4:CD8 human T-Cells were thawed and activated with CD3/28 Dynabeads® (1:3 cells:bead ratio) in X-vivo15 T-cell culture media (day 0).

After 3 days in culture (day 3), cells were concentrated to 3e7 cells/mL in Maxcyte electroporation buffer in the presence of ZFN mRNA, then were electroporated using the Maxcyte device. Concentrated, electroporated cells were then placed in a tissue culture well, then AAV6 encoding for a hPGK-GFP-BGHpolyA transgene donor was added to the concentrated cells, which were allowed to recover and incubate at 37° C. for 20 minutes. Alternatively, the donor vector can be added to the electroporation buffer in the device. Cells were then diluted in culture medium to 3e6 cells/mL and cultured at 30° C. overnight. The next morning cells were diluted to 0.5e6 cells/mL in additional culture medium. The following is a description of the groups:
(a) Sham: cells electroporated with no ZFN mRNA or AAV donor added;
(b) TRAC and B2M ZFNs only, no donor): TRAC 120 ug/mL: B2M only 30 ug/mL;
(c) TRAC and B2M ZFNs and donor with B2M homology arms: TRAC 120 ug/mL; B2M 30 ug/mL and AAV6 (B2M-Site A-hPGK-eGFP) 3E4 vg/cell;
(d) TCAC and B2M ZFNs and donor with TRAC homology arms: TRAC 120 ug/mL; B2M 30 ug/mL; and AAV6 (TCRA-Site E-hPGK-eGFP) 3E4 vg/cell.

All experiments were conducted at 3e7 cells/ml cell density using the protocol as described in U.S. Pat. Publication No. 2017/0137845 (extreme cold shock) and were cultured to cold shock at 30° C. overnight post electroporation. The following day (day 4), cells were diluted to 0.5e6 cells/mL and transferred to cultures at 37 C. Three days later (day 7), cells diluted to 0.5e6 cells/mL again. After three and seven more days in culture (days 10 and 14, respectively), cells were harvested for FACS and MiSeq analysis (diluted to 0.5e6 cells/mL).

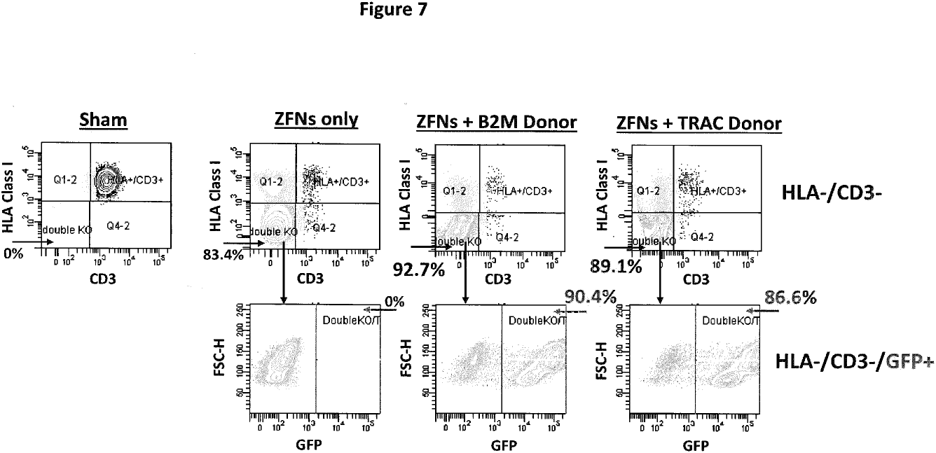

As shown in FIG. 7, GFP expression (donor) indicated that target integration was successful and that genetically modified cells comprising B2M and TRAC modifications (insertions and/or deletions) within the nuclease target sites (or within 1 to 50, 1-20, 1-10 or 1-5 base pairs of the nuclease target sites, including between paired sites) as disclosed herein were obtained with high frequency (including 80-90% knockout and targeted integration rates).

Experiments are also performed in which a CAR transgene is integrated into B2M and TCRA double-knockouts, either at the B2M, TCRA or another locus to created double B2M/TCRA knockouts that express a CAR.

### Example 5: Optimization of TCRA and B2M ZFNs

To decrease off target cleavage, a strategy for nuclease optimization in which nonspecific phosphate contacts are selectively removed to bring about global suppression off-target cleavage (Guilinger, et al. (2014) NatMethods. 11(4):429-35. doi: 10.1038/nmeth.2845; Kleinstiver, et al. (2016) Nature 529(7587):490-5. doi: 10.1038/nature16526; Slaymaker, et al. (2016) Science) 351(6268):84-8. doi: 10.1126/science.aad5227) was adopted (see U.S. Pat. Publication No. 2018/0087072). Amino acid substitutions were made at one or more key positions within the zinc finger framework that interacts with the phosphate backbone of the DNA (Pavletich and Pabo (1991) Science 252(5007):809-17; Elrod-Erickson, et al. (1996) Structure 4(10): 1171-80) as well as at positions in the right ZFN FokI domain also predicted to make a phosphate contact.

In Table 6 below, characterizing information for each ZFN is shown. Starting from the left, the SBS number (e.g., 55254) is displayed with the DNA target that the ZFN binds to displayed below the SBS number. Next are shown the amino acid recognition helix designs for fingers 1-6 or 1-5 (subdivided column 2 of Table 6). Also shown in Table 6 under the appropriate helix designs are mutations made to the ZFP backbone sequences of the indicated finger, as described in U.S. Pat. Publication No. 2018/0087072. In the notation used in Table 6, “Qm5” means that at position minus 5 (relative to the helix which is numbered -1 to +6) of the indicated finger, the arginine at this position has been replaced with a glutamine (Q), while “Qm14” means that the arginine (R) normally present in position minus 14 has been replaced with a glutamine (Q). The abbreviation “n” as in nQm5 means that the mutation is in the N-terminal finger of the two-finger module used in the build of the 5 or 6 fingered protein. “None” indicates no changes outside the recognition helix region. Thus, for example, SBS# 68797 includes the nQm5 mutation in fingers 1, 3 and 5 while fingers 2, 4 and 6 do not have mutations to the zinc finger backbone (e.g., the zinc finger sequence outside the recognition helix region).

Finally, the right-most column of Table 6 shows the linker used to link the DNA binding domain to the FokI cleavage domain (e.g., “L0” LRGSQLVKS (SEQ ID NO: 135), as referred to as the ‘standard’ linker, and described for example in U.S. Pat. No. 9,567,609) is displayed on top line of the column, with the sites of the FokI phosphate contact mutations and dimerization mutations shown in the box below the linker designation. Other linkers include N7c (SGAIRCHDEFWF, SEQ ID NO: 136) and N7a (SGTPHEVGVYTL, SEQ ID NO: 137). In specifics, indicated on top line of the Fok mutants box is the type of mutation found in the dimerizing domain (e.g., ELD or KKR as described for example in U.S. Pat. No. 8,962,281). Below the dimerization mutant designations is shown any mutations present in the FokI domain made to remove a non-specific phosphate contact shown on the bottom (e.g., K525S or R416S where serine residues at amino acid positions 525 or 416 have been substituted for either a lysine or arginine, respectively as described in U.S. Publication No. 20180087072). Thus, for example, in SBS# 68796, the linker is an L0 linker and the FokI cleavage domain includes the ELD dimerization mutants and no phosphate contact mutations. Further, for SBS# 68812, the linker is an L0 linker and the FokI cleavage domain includes the KKR dimerization mutations where the FokI domain further comprises an R416E substitutional mutation.

Other FokI domain variants that may be used with the ZFPs described herein (including ZFPs derived from the ZFNs described herein) include the addition of a Sharkey mutation (S418P+K441E, see Guo, et al. (2010) J. Mol Biol, doi:10.1016/j.jmb.2010.04.060) and the DAD and RVR FokI mutations (see U.S. Pat. No. 8,962,281). Non-limiting examples of engineered FokI variants that may be used include:

• Wildtype FokI cleavage domain(SEQ ID NO:139) :
QLVKSELEEK KSELRHKLKY VPHEYIELIE IARNSTQDRI LEMKVMEFFM 384- 433
KVYGYRGKHL GGSRKPDGAI YTVGSPIDYG VIVDTKAYSG GYNLPIGQAD 434- 483
EMQRYVEENQ TRNKHINPNE WWKVYPSSVT EFKFLFVSGH FKGNYKAQLT 484- 533
RLNHITNCNG AVLSVEELLI GGEMIKAGTL TLEEVRRKFN NGEINF 534- 579

• FokI-Sharkey (S418P+K441E, SEQ ID NO:140):
QLVKSELEEK KSELRHKLKY VPHEYIELIE IARNPTQDRI LEMKVMEFFM 384- 433
KVYGYRGEHL GGSRKPDGAI YTVGSPIDYG VIVDTKAYSG GYNLPIGQAD 434- 483
EMQRYVEENQ TRNKHINPNE WWKVYPSSVT EFKFLFVSGH FKGNYKAQLT 484- 533
RLNHITNCNG AVLSVEELLI GGEMIKAGTL TLEEVRRKFN NGEINF 534- 579

• FokI ELD (Q->E @ 486, I->L @499, N->D @496, SEQ ID NO:141)
QLVKSELEEK KSELRHKLKY VPHEYIELIE IARNSTQDRI LEMKVMEFFM 384- 433
KVYGYRGKHL GGSRKPDGAI YTVGSPIDYG VIVDTKAYSG GYNLPIGQAD 434- 483
EMERYVEENQ TRDKHLNPNE WWKVYPSSVT EFKFLFVSGH FKGNYKAQLT 484- 533
RLNHITNCNG AVLSVEELLI GGEMIKAGTL TLEEVRRKFN NGEINF 534- 579

• FokI ELD, Sharkey (Q->E @ 486, I->L @499, N->D @496, S418P+K441E SEQ ID
NO:142)
QLVKSELEEK KSELRHKLKY VPHEYIELIE IARNPTQDRI LEMKVMEFFM 384- 433
KVYGYRGEHL GGSRKPDGAI YTVGSPIDYG VIVDTKAYSG GYNLPIGQAD 434- 483
EMERYVEENQ TRDKHLNPNE WWKVYPSSVT EFKFLFVSGH FKGNYKAQLT 484- 533
RLNHITNCNG AVLSVEELLI GGEMIKAGTL TLEEVRRKFN NGEINF 534- 579

• FokI ELD, R416E (Q->E @ 486, I->L @499, N->D @496, R416E, SEQ ID NO:143)
QLVKSELEEK KSELRHKLKY VPHEYIELIE IAENSTQDRI LEMKVMEFFM 384- 433
KVYGYRGKHL GGSRKPDGAI YTVGSPIDYG VIVDTKAYSG GYNLPIGQAD 434- 483
EMERYVEENQ TRDKHLNPNE WWKVYPSSVT EFKFLFVSGH FKGNYKAQLT 484- 533
RLNHITNCNG AVLSVEELLI GGEMIKAGTL TLEEVRRKFN NGEINF 534- 579

• FokI ELD, Sharkey, R416E (Q->E @ 486, I->L @499, N->D @496, S418P+K441E,
R416E, SEQ ID NO:144)
QLVKSELEEK KSELRHKLKY VPHEYIELIE IAENPTQDRI LEMKVMEFFM 384- 433
KVYGYRGEHL GGSRKPDGAI YTVGSPIDYG VIVDTKAYSG GYNLPIGQAD 434- 483
EMERYVEENQ TRDKHLNPNE WWKVYPSSVT EFKFLFVSGH FKGNYKAQLT 484- 533
RLNHITNCNG AVLSVEELLI GGEMIKAGTL TLEEVRRKFN NGEINF 534- 579

• FokI ELD, R416Y (Q->E @ 486, I->L @499, N->D @496, R416Y, SEQ ID NO:145)
QLVKSELEEK KSELRHKLKY VPHEYIELIE IAYNSTQDRI LEMKVMEFFM 384- 433
KVYGYRGKHL GGSRKPDGAI YTVGSPIDYG VIVDTKAYSG GYNLPIGQAD 434- 483
EMERYVEENQ TRDKHLNPNE WWKVYPSSVT EFKFLFVSGH FKGNYKAQLT 484- 533
RLNHITNCNG AVLSVEELLI GGEMIKAGTL TLEEVRRKFN NGEINF 534- 579

• FokI ELD, Sharkey, R416E (Q->E @ 486, I->L @499, N->D @496, S418P+K441E,
R416E, SEQ ID NO:146)
QLVKSELEEK KSELRHKLKY VPHEYIELIE IAYNPTQDRI LEMKVMEFFM 384- 433
KVYGYRGEHL GGSRKPDGAI YTVGSPIDYG VIVDTKAYSG GYNLPIGQAD 434- 483
EMERYVEENQ TRDKHLNPNE WWKVYPSSVT EFKFLFVSGH FKGNYKAQLT 484- 533
RLNHITNCNG AVLSVEELLI GGEMIKAGTL TLEEVRRKFN NGEINF 534- 579

• FokI ELD, S418E (Q->E @ 486, I->L @499, N->D @496, S418E, SEQ ID NO:147)
QLVKSELEEK KSELRHKLKY VPHEYIELIE IARNETQDRI LEMKVMEFFM 384- 433
KVYGYRGKHL GGSRKPDGAI YTVGSPIDYG VIVDTKAYSG GYNLPIGQAD 434- 483
EMERYVEENQ TRDKHLNPNE WWKVYPSSVT EFKFLFVSGH FKGNYKAQLT 484- 533
RLNHITNCNG AVLSVEELLI GGEMIKAGTL TLEEVRRKFN NGEINF 534- 579

• FokI ELD, Sharkey partial, S418E (Q->E @ 486, I->L @499, N->D @496,
K441E, S418E, SEQ ID NO:148)
QLVKSELEEK KSELRHKLKY VPHEYIELIE IARNETQDRI LEMKVMEFFM 384- 433
KVYGYRGEHL GGSRKPDGAI YTVGSPIDYG VIVDTKAYSG GYNLPIGQAD 434- 483
EMERYVEENQ TRDKHLNPNE WWKVYPSSVT EFKFLFVSGH FKGNYKAQLT 484- 533
RLNHITNCNG AVLSVEELLI GGEMIKAGTL TLEEVRRKFN NGEINF 534- 579

• FokI ELD, K525S (Q->E @ 486, I->L @499, N->D @496, K525S, SEQ ID NO:149)
QLVKSELEEK KSELRHKLKY VPHEYIELIE IARNSTQDRI LEMKVMEFFM 384- 433
KVYGYRGKHL GGSRKPDGAI YTVGSPIDYG VIVDTKAYSG GYNLPIGQAD 434- 483
EMERYVEENQ TRDKHLNPNE WWKVYPSSVT EFKFLFVSGH FSGNYKAQLT 484- 533
RLNHITNCNG AVLSVEELLI GGEMIKAGTL TLEEVRRKFN NGEINF 534- 579

• FokI ELD, Sharkey K525S (Q->E @ 486, I->L @499, N->D @496, S418P+K441E,
K525S, SEQ ID NO:150)
QLVKSELEEK KSELRHKLKY VPHEYIELIE IARNPTQDRI LEMKVMEFFM 384- 433
KVYGYRGEHL GGSRKPDGAI YTVGSPIDYG VIVDTKAYSG GYNLPIGQAD 434- 483
EMERYVEENQ TRDKHLNPNE WWKVYPSSVT EFKFLFVSGH FSGNYKAQLT 484- 533
RLNHITNCNG AVLSVEELLI GGEMIKAGTL TLEEVRRKFN NGEINF 534- 579

• FokI ELD, I479T (Q->E @ 486, I->L @499, N->D @496, I479T, SEQ ID NO:151)
QLVKSELEEK KSELRHKLKY VPHEYIELIE IARNSTQDRI LEMKVMEFFM 384- 433
KVYGYRGKHL GGSRKPDGAI YTVGSPIDYG VIVDTKAYSG GYNLPTGQAD 434- 483
EMERYVEENQ TRDKHLNPNE WWKVYPSSVT EFKFLFVSGH FKGNYKAQLT 484- 533
RLNHITNCNG AVLSVEELLI GGEMIKAGTL TLEEVRRKFN NGEINF 534- 579

• FokI ELD, Sharkey, I479T (Q->E @ 486, I->L @499, N->D @496, S418P+K441E,
I479T, SEQ ID NO:152)
QLVKSELEEK KSELRHKLKY VPHEYIELIE IARNPTQDRI LEMKVMEFFM 384- 433
KVYGYRGEHL GGSRKPDGAI YTVGSPIDYG VIVDTKAYSG GYNLPTGQAD 434- 483
EMERYVEENQ TRDKHLNPNE WWKVYPSSVT EFKFLFVSGH FKGNYKAQLT 484- 533
RLNHITNCNG AVLSVEELLI GGEMIKAGTL TLEEVRRKFN NGEINF 534- 579

• FokI ELD, P478D (Q->E @ 486, I->L @499, N->D @496, P478D, SEQ ID NO:153)
QLVKSELEEK KSELRHKLKY VPHEYIELIE IARNSTQDRI LEMKVMEFFM 384- 433
KVYGYRGKHL GGSRKPDGAI YTVGSPIDYG VIVDTKAYSG GYNLDIGQAD 434- 483
EMERYVEENQ TRDKHLNPNE WWKVYPSSVT EFKFLFVSGH FKGNYKAQLT 484- 533
RLNHITNCNG AVLSVEELLI GGEMIKAGTL TLEEVRRKFN NGEINF 534- 579

• FokI ELD, Sharkey, P478D (Q->E @ 486, I->L @499, N->D @496, S418P+K441E,
P478D, SEQ ID NO:154)
QLVKSELEEK KSELRHKLKY VPHEYIELIE IARNPTQDRI LEMKVMEFFM 384- 433
KVYGYRGEHL GGSRKPDGAI YTVGSPIDYG VIVDTKAYSG GYNLDIGQAD 434- 483
EMERYVEENQ TRDKHLNPNE WWKVYPSSVT EFKFLFVSGH FKGNYKAQLT 484- 533
RLNHITNCNG AVLSVEELLI GGEMIKAGTL TLEEVRRKFN NGEINF 534- 579

• FokI ELD, Q481D (Q->E @ 486, I->L @499, N->D @496, Q481D, SEQ ID NO:155)
QLVKSELEEK KSELRHKLKY VPHEYIELIE IARNSTQDRI LEMKVMEFFM 384- 433
KVYGYRGKHL GGSRKPDGAI YTVGSPIDYG VIVDTKAYSG GYNLPIGDAD 434- 483
EMERYVEENQ TRDKHLNPNE WWKVYPSSVT EFKFLFVSGH FKGNYKAQLT 484- 533
RLNHITNCNG AVLSVEELLI GGEMIKAGTL TLEEVRRKFN NGEINF 534- 579

• FokI ELD, Sharkey, Q481D (Q->E @ 486, I->L @499, N->D @496, S418P+K441E,
Q481D, SEQ ID NO:156)
QLVKSELEEK KSELRHKLKY VPHEYIELIE IARNPTQDRI LEMKVMEFFM 384- 433
KVYGYRGEHL GGSRKPDGAI YTVGSPIDYG VIVDTKAYSG GYNLPIGDAD 434- 483
EMERYVEENQ TRDKHLNPNE WWKVYPSSVT EFKFLFVSGH FKGNYKAQLT 484- 533
RLNHITNCNG AVLSVEELLI GGEMIKAGTL TLEEVRRKFN NGEINF 534- 579

• FokI KKR (E->K @490, I->K@538, H->R@537, SEQ ID NO:157)
QLVKSELEEK KSELRHKLKY VPHEYIELIE IARNSTQDRI LEMKVMEFFM 384- 433
KVYGYRGKHL GGSRKPDGAI YTVGSPIDYG VIVDTKAYSG GYNLPIGQAD 434- 483
EMQRYVKENQ TRNKHINPNE WWKVYPSSVT EFKFLFVSGH FKGNYKAQLT 484- 533
RLNRKTNCNG AVLSVEELLI GGEMIKAGTL TLEEVRRKFN NGEINF 534- 579

• FokI KKR Sharkey, (E->K @490, I->K@538, H->R@537, S418P+K441E, SEQ ID
NO:158)
QLVKSELEEK KSELRHKLKY VPHEYIELIE IARNPTQDRI LEMKVMEFFM 384- 433
KVYGYRGEHL GGSRKPDGAI YTVGSPIDYG VIVDTKAYSG GYNLPIGQAD 434- 483
EMQRYVKENQ TRNKHINPNE WWKVYPSSVT EFKFLFVSGH FKGNYKAQLT 484- 533
RLNRKTNCNG AVLSVEELLI GGEMIKAGTL TLEEVRRKFN NGEINF 534- 579

• FokI KKR, Q481E (E->K @490, I->K@538, H->R@537, Q481E, SEQ ID NO:159)
QLVKSELEEK KSELRHKLKY VPHEYIELIE IARNSTQDRI LEMKVMEFFM 384- 433
KVYGYRGKHL GGSRKPDGAI YTVGSPIDYG VIVDTKAYSG GYNLPIGEAD 434- 483
EMQRYVKENQ TRNKHINPNE WWKVYPSSVT EFKFLFVSGH FKGNYKAQLT 484- 533
RLNRKTNCNG AVLSVEELLI GGEMIKAGTL TLEEVRRKFN NGEINF 534- 579

• FokI KKR, Sharkey Q481E (E->K @490, I->K@538, H->R@537, S418P+K441E,
Q481E, SEQ ID NO:160)
QLVKSELEEK KSELRHKLKY VPHEYIELIE IARNPTQDRI LEMKVMEFFM 384- 433
KVYGYRGEHL GGSRKPDGAI YTVGSPIDYG VIVDTKAYSG GYNLPIGEAD 434- 483
EMQRYVKENQ TRNKHINPNE WWKVYPSSVT EFKFLFVSGH FKGNYKAQLT 484- 533
RLNRKTNCNG AVLSVEELLI GGEMIKAGTL TLEEVRRKFN NGEINF 534- 579

• FokI KKR, R416E (E->K @490, I->K@538, H->R@537, R416E, SEQ ID NO:161)
QLVKSELEEK KSELRHKLKY VPHEYIELIE IAENSTQDRI LEMKVMEFFM 384- 433
KVYGYRGKHL GGSRKPDGAI YTVGSPIDYG VIVDTKAYSG GYNLPIGQAD 434- 483
EMQRYVKENQ TRNKHINPNE WWKVYPSSVT EFKFLFVSGH FKGNYKAQLT 484- 533
RLNRKTNCNG AVLSVEELLI GGEMIKAGTL TLEEVRRKFN NGEINF 534- 579

• FokI KKR, Sharkey, R416E (E->K @490, I->K@538, H->R@537, S418P+K441E,
R416E, SEQ ID NO:162)
QLVKSELEEK KSELRHKLKY VPHEYIELIE IAENPTQDRI LEMKVMEFFM 384- 433
KVYGYRGEHL GGSRKPDGAI YTVGSPIDYG VIVDTKAYSG GYNLPIGQAD 434- 483
EMQRYVKENQ TRNKHINPNE WWKVYPSSVT EFKFLFVSGH FKGNYKAQLT 484- 533
RLNRKTNCNG AVLSVEELLI GGEMIKAGTL TLEEVRRKFN NGEINF 534- 579

• FokI KKR, K525S (E->K @490, I->K@538, H->R@537, K525S, SEQ ID NO:163)
QLVKSELEEK KSELRHKLKY VPHEYIELIE IARNSTQDRI LEMKVMEFFM 384- 433
KVYGYRGKHL GGSRKPDGAI YTVGSPIDYG VIVDTKAYSG GYNLPIGQAD 434- 483
EMQRYVKENQ TRNKHINPNE WWKVYPSSVT EFKFLFVSGH FSGNYKAQLT 484- 533
RLNRKTNCNG AVLSVEELLI GGEMIKAGTL TLEEVRRKFN NGEINF 534- 579

• FokI KKR, Sharkey, K525S (E->K @490, I->K@538, H->R@537, S418P+K441E,
K525S, SEQ ID NO:164)
QLVKSELEEK KSELRHKLKY VPHEYIELIE IARNPTQDRI LEMKVMEFFM 384- 433
KVYGYRGEHL GGSRKPDGAI YTVGSPIDYG VIVDTKAYSG GYNLPIGQAD 434- 483
EMQRYVKENQ TRNKHINPNE WWKVYPSSVT EFKFLFVSGH FSGNYKAQLT 484- 533
RLNRKTNCNG AVLSVEELLI GGEMIKAGTL TLEEVRRKFN NGEINF 534- 579

• FokI KKR, R416Y (E->K @490, I->K@538, H->R@537, R416Y, SEQ ID NO:165)
QLVKSELEEK KSELRHKLKY VPHEYIELIE IAYNSTQDRI LEMKVMEFFM 384- 433
KVYGYRGKHL GGSRKPDGAI YTVGSPIDYG VIVDTKAYSG GYNLPIGQAD 434- 483
EMQRYVKENQ TRNKHINPNE WWKVYPSSVT EFKFLFVSGH FKGNYKAQLT 484- 533
RLNRKTNCNG AVLSVEELLI GGEMIKAGTL TLEEVRRKFN NGEINF 534- 579

• FokI KKR, Sharkey, R416Y (E->K @490, I->K@538, H->R@537, S418P+K441E,
R416Y, SEQ ID NO:166)
QLVKSELEEK KSELRHKLKY VPHEYIELIE IAYNPTQDRI LEMKVMEFFM 384- 433
KVYGYRGEHL GGSRKPDGAI YTVGSPIDYG VIVDTKAYSG GYNLPIGQAD 434- 483
EMQRYVKENQ TRNKHINPNE WWKVYPSSVT EFKFLFVSGH FKGNYKAQLT 484- 533
RLNRKTNCNG AVLSVEELLI GGEMIKAGTL TLEEVRRKFN NGEINF 534- 579

• FokI, KKR I479T (E->K @490, I->K@538, H->R@537, I479T, SEQ ID NO:167)
QLVKSELEEK KSELRHKLKY VPHEYIELIE IARNSTQDRI LEMKVMEFFM 384- 433
KVYGYRGKHL GGSRKPDGAI YTVGSPIDYG VIVDTKAYSG GYNLPTGQAD 434- 483
EMQRYVKENQ TRNKHINPNE WWKVYPSSVT EFKFLFVSGH FKGNYKAQLT 484- 533
RLNRKTNCNG AVLSVEELLI GGEMIKAGTL TLEEVRRKFN NGEINF 534- 579

• FokI, KKR Sharkey I479T (E->K @490, I->K@538, H->R@537, S418P+K441E,
I479T, SEQ ID NO:168)
QLVKSELEEK KSELRHKLKY VPHEYIELIE IARNPTQDRI LEMKVMEFFM 384- 433
KVYGYRGEHL GGSRKPDGAI YTVGSPIDYG VIVDTKAYSG GYNLPTGQAD 434- 483
EMQRYVKENQ TRNKHINPNE WWKVYPSSVT EFKFLFVSGH FKGNYKAQLT 484- 533
RLNRKTNCNG AVLSVEELLI GGEMIKAGTL TLEEVRRKFN NGEINF 534- 579

• FokI, KKR P478D(E->K @490, I->K@538, H->R@537, P478D, SEQ ID NO:169)
QLVKSELEEK KSELRHKLKY VPHEYIELIE IARNSTQDRI LEMKVMEFFM 384- 433
KVYGYRGKHL GGSRKPDGAI YTVGSPIDYG VIVDTKAYSG GYNLDIGQAD 434- 483
EMQRYVKENQ TRNKHINPNE WWKVYPSSVT EFKFLFVSGH FKGNYKAQLT 484- 533
RLNRKTNCNG AVLSVEELLI GGEMIKAGTL TLEEVRRKFN NGEINF 534- 579

• FokI, KKR Sharkey P478D(E->K @490, I->K@538, H->R@537, P478D, SEQ ID
NO:170)
QLVKSELEEK KSELRHKLKY VPHEYIELIE IARNPTQDRI LEMKVMEFFM 384- 433
KVYGYRGEHL GGSRKPDGAI YTVGSPIDYG VIVDTKAYSG GYNLDIGQAD 434- 483
EMQRYVKENQ TRNKHINPNE WWKVYPSSVT EFKFLFVSGH FKGNYKAQLT 484- 533
RLNRKTNCNG AVLSVEELLI GGEMIKAGTL TLEEVRRKFN NGEINF 534- 579

• FokI DAD (R->D@487, N->D@496, I->A@499, SEQ ID NO:171)
QLVKSELEEK KSELRHKLKY VPHEYIELIE IARNSTQDRI LEMKVMEFFM 384- 433
KVYGYRGKHL GGSRKPDGAI YTVGSPIDYG VIVDTKAYSG GYNLPIGQAD 434- 483
EMQDYVEENQ TRDKHANPNE WWKVYPSSVT EFKFLFVSGH FKGNYKAQLT 484- 533
RLNHITNCNG AVLSVEELLI GGEMIKAGTL TLEEVRRKFN NGEINF 534- 579

• FokI DAD Sharkey (R->D@487, N->D@496, I->A@499, S418P+K441E, SEQ ID
NO:172)
QLVKSELEEK KSELRHKLKY VPHEYIELIE IARNPTQDRI LEMKVMEFFM 384- 433
KVYGYRGEHL GGSRKPDGAI YTVGSPIDYG VIVDTKAYSG GYNLPIGQAD 434- 483
EMQDYVEENQ TRDKHANPNE WWKVYPSSVT EFKFLFVSGH FKGNYKAQLT 484- 533
RLNHITNCNG AVLSVEELLI GGEMIKAGTL TLEEVRRKFN NGEINF 534- 579

• FokI RVR (D->R@483, H->R@537, I->V@538, SEQ ID NO:173)
QLVKSELEEK KSELRHKLKY VPHEYIELIE IARNSTQDRI LEMKVMEFFM 384- 433
KVYGYRGKHL GGSRKPDGAI YTVGSPIDYG VIVDTKAYSG GYNLPIGQAR 434- 483
EMQRYVEENQ TRNKHINPNE WWKVYPSSVT EFKFLFVSGH FKGNYKAQLT 484- 533
RLNRVTNCNG AVLSVEELLI GGEMIKAGTL TLEEVRRKFN NGEINF 534- 579

• FokI RVR Sharkey (D->R@483, H->R@537, I->V@538, S418P+K441E, SEQ ID
NO:174)
QLVKSELEEK KSELRHKLKY VPHEYIELIE IARNPTQDRI LEMKVMEFFM 384- 433
KVYGYRGEHL GGSRKPDGAI YTVGSPIDYG VIVDTKAYSG GYNLPIGQAR 434- 483
EMQRYVEENQ TRNKHINPNE WWKVYPSSVT EFKFLFVSGH FKGNYKAQLT 484- 533
RLNRVTNCNG AVLSVEELLI GGEMIKAGTL TLEEVRRKFN NGEINF 534- 579

All pairwise combinations of ZFNs were tested for functionality and all were found to be active.

TABLE 6

ZFN pairs specific for TCRA

| SBS # (target site, 5′-3′) | Design [Helix Sequence, SEQ ID] | | | | | | Linker |
|---|---|---|---|---|---|---|---|
| | [Mutations to finger backbone] | | | | | | Fok mutants |
| | F1 | F2 | F3 | F4 | F5 | F6 | |
| Site D | | | | | | | |
| Left partner | | | | | | | |
| 55254 5′ ctCC TGAAAGT GGCCGGg tttaatc tgc (SEQ ID NO: 13) | RSDHLST (SEQ ID NO:47) | DRSHLAR (SEQ ID NO:48) | LKQHLNE (SEQ ID NO:49) | QSGNLAR (SEQ ID NO:34) | HNSSLKD (SEQ ID NO:54) | N/A | L0 |
| | none | none | none | none | none | N/A | ELD C-term Fok |
| 68796 ctCCTGA AAGTGGC CGGgttt aatctgc (SEQ ID NO:13) | RSDHLST (SEQ ID NO:47) | DRSHLAR (SEQ ID NO:48) | LKQHLNE (SEQ ID NO:49) | QSGNLAR (SEQ ID NO:34) | HNSSLKD (SEQ ID NO:54) | N/A | L0 |
| | nQm5 | none | nQm5s | nQm5 | none | N/A | ELD C-term Fok |
| 68812 ctCCTGA AAGTGGC CGGgttt aatctgc (SEQ ID NO:13) | RSDHLST (SEQ ID NO:47) | DRSHLAR (SEQ ID NO:48) | LKQHLNE (SEQ ID NO:49) | QSGNLAR (SEQ ID NO:34) | HNSSLKD (SEQ ID NO:54) | N/A | L0 |
| | nQm5 | none | nQm5s | nQm5 | none | N/A | ELD R416E C-term Fok |
| 68820 ctCCTGA AAGTGGC CGGgttt aatctgc (SEQ ID NO:13) | RSDHLST (SEQ ID NO:47) | DRSHLAR (SEQ ID NO:48) | LKQHLNE (SEQ ID NO:49) | QSGNLAR (SEQ ID NO:34) | HNSSLKD (SEQ ID NO:54) | N/A | L0 |
| | none | none | none | none | none | N/A | ELD S418E C-term Fok |
| 68876 ctCCTGA AAGTGGC | RSDHLST (SEQ ID NO:47) | DRSHLAR (SEQ ID NO:48) | LKQHLNE (SEQ ID NO:49) | QSGNLAR (SEQ ID NO:34) | HNSSLKD (SEQ ID NO:54) | N/A | L0 |
| CGGgttt aatctgc (SEQ ID NO:13) | nQm5 | none | nQm5s | nQm5 | none | N/A | ELD K525S C-term Fok |
| 55248 5′agGAT TCGGAAC CCAATCA Ctgacag gt(SEQ ID NO:14) | DQSNLRA (SEQ ID NO:42) | TSSNRKT (SEQ ID NO:43) | LQQTLAD (SEQ ID NO:51) | QSGNLAR (SEQ ID NO:34) | RREDLIT (SEQ ID NO:52) | TSSNLSR (SEQ ID NO:53) | L0 |
| | none | none | none | none | none | none | KKR C-term Fok |
| 68797 agGATTC GGAACCC AATCACt gacaggt (SEQ ID NO:14) | DQSNLRA (SEQ ID NO:42) | TSSNRKT (SEQ ID NO:43) | LQQTLAD (SEQ ID NO:51) | QSGNLAR (SEQ ID NO:34) | RREDLIT (SEQ ID NO:52) | TSSNLSR (SEQ ID NO:53) | L0 |
| | nQm5 | none | nQm5 | none | nQm5 | none | KKR C-term Fok |
| 68813 agGATTC GGAACCC AATCACt gacaggt (SEQ ID NO:14) | DQSNLRA (SEQ ID NO:42) | TSSNRKT (SEQ ID NO:43) | LQQTLAD (SEQ ID NO:51) | QSGNLAR (SEQ ID NO:34) | RREDLIT (SEQ ID NO:52) | TSSNLSR (SEQ ID NO:53) | L0 |
| | nQm5 | none | nQm5 | none | nQm5 | none | KKR R416E C-term Fok |
| 68861 agGATTC GGAACCC AATCACt gacaggt (SEQ ID NO:14) | DQSNLRA (SEQ ID NO:42) | TSSNRKT (SEQ ID NO:43) | LQQTLAD (SEQ ID NO:51) | QSGNLAR (SEQ ID NO:34) | RREDLIT (SEQ ID NO:52) | TSSNLSR (SEQ ID NO:53) | L0 |
| | nQm5 | none | nQm5 | none | nQm5 | none | KKR Q481E C-term Fok |
| 68877 agGATTC GGAACCC AATCACt gacaggt (SEQ ID NO:14) | DQSNLRA (SEQ ID NO:42) | TSSNRKT (SEQ ID NO:43) | LQQTLAD (SEQ ID NO:51) | QSGNLAR (SEQ ID NO:34) | RREDLIT (SEQ ID NO:52) | TSSNLSR (SEQ ID NO:53) | L0 |
| | nQm5 | none | nQm5 | none | nQm5 | none | KKR K525S C-term Fok |
| 55266 tcAAGCT GGTCGAG aAAAGCT ttgaaac (SEQ ID NO:15) | QSSDLSR (SEQ ID NO:57) | QSGNRTT (SEQ ID NO:58) | RSANLAR (SEQ ID NO: 59) | DRSALAR (SEQ ID NO:33) | RSDVLSE (SEQ ID NO: 60) | KHSTRRV (SEQ ID NO:61) | N7c |
| | none | none | none | none | none | none | ELD N-term Fok |
| 68798 tcAAGCT GGTCGAG aAAAGCT ttgaaac | QSSDLSR (SEQ ID NO:57) | QSGNRTT (SEQ ID NO:58) | RSANLAR (SEQ ID NO: 59) | DRSALAR (SEQ ID NO:33) | RSDVLSE (SEQ ID NO: 60) | KHSTRRV (SEQ ID NO:61) | N7c |
| | nQm5 | none | nQm5 | none | nQm5 | none | ELD N-term |
| (SEQ ID NO:15) | | | | | | | Fok |
| 68846 tcAAGCT GGTCGAG aAAAGCT ttgaaac (SEQ ID NO:15) | QSSDLSR (SEQ ID NO:57) | QSGNRTT (SEQ ID NO:58) | RSANLAR (SEQ ID NO: 59) | DRSALAR (SEQ ID NO:33) | RSDVLSE (SEQ ID NO: 60) | KHSTRRV (SEQ ID NO:61) | N7c |
| | nQm5 | none | nQm5 | none | nQm5 | none | ELD I479T N-term Fok |
| 53853 aaCAGGT AaGACAG GGGTCTA gcctggg (SEQ ID NO:16) | TMHQRVE (SEQ ID NO:62) | TSGHLSR (SEQ ID NO:63) | RSDHLTQ (SEQ ID NO:64) | DSANLSR (SEQ ID NO:65) | QSGSLTR (SEQ ID NO:66) | AKWNLDA (SEQ ID NO:67) | L0 |
| | none | none | none | none | none | none | KKR C-term Fok |
| 68879 aaCAGGT AaGACAG GGGTCTA gcctggg (SEQ ID NO:16) | TMHQRVE (SEQ ID NO:62) | TSGHLSR (SEQ ID NO:63) | RSDHLTQ (SEQ ID NO:64) | DSANLSR (SEQ ID NO:65) | QSGSLTR (SEQ ID NO:66) | AKWNLDA (SEQ ID NO:67) | L0 |
| | nQm5 | none | nQm5 | none | nQm5 | none | KKR K525S C-term Fok |
| 68815 aaCAGGT AaGACAG GGGTCTA gcctggg (SEQ ID NO:16) | TMHQRVE (SEQ ID NO:62) | TSGHLSR (SEQ ID NO:63) | RSDHLTQ (SEQ ID NO:64) | DSANLSR (SEQ ID NO:65) | QSGSLTR (SEQ ID NO:66) | AKWNLDA (SEQ ID NO:67) | L0 |
| | nQm5 | none | nQm5 | none | nQm5 | none | KKR R416E C-term Fok |
| 68799 aaCAGGT AaGACAG GGGTCTA gcctggg (SEQ ID NO:16) | TMHQRVE (SEQ ID NO:62) | TSGHLSR (SEQ ID NO:63) | RSDHLTQ (SEQ ID NO:64) | DSANLSR (SEQ ID NO:65) | QSGSLTR (SEQ ID NO:66) | AKWNLDA (SEQ ID NO:67) | L0 |
| | nQm5 | none | nQm5 | none | nQm5 | none | KKR C-term Fok |

Genes encoding the ZFNs for each site were cloned into an expression plasmid as right and left partners separated by a 2A self-cleaving peptide in combinations for each target site. mRNA encoding the ZFNs were derived using standard in vitro transcription methods. Activated T cells (3 days post activation) were then treated with the various mRNAs at 3 different doses (12, 6 or 3 µg in 100 µL, 3E6 T-cells) by electroporation. 4 days post electroporation, the cells were analyzed for cleavage at the target sites and at the target site. The data are presented below in two tables (one for each target site).

TABLE 7a

On Target and Off Target cleavage at Site D

| | SITE D | 55254--2A-55248 | 68796-2A-68813 | 68813-2A-68796 | 68796-2A-68861 | 6886 1-2A-6879 6 | 68812-2A-68813 | 68813-2A-68812 | 68876-2A-68877 | 68877-2A-68876 | Control |
|---|---|---|---|---|---|---|---|---|---|---|---|
| On Target | 12ug | 96.7 | 99.3 | 98.8 | 99.4 | 99.3 | 99.9 | 99.9 | 99.2 | 99.1 | 0.12 |
| | 6ug | 98.5 | 99.2 | 99.1 | 99.4 | 99.4 | 99 | 98.9 | 99.3 | 99.2 | 0.14 |
| | 3ug | 96 | 99.1 | 98.8 | 99.3 | 98.9 | 98.3 | 97.8 | 98.7 | 99.3 | 0.15 |
| Off D1 | 12ug | 39.6 | 0.29 | 0.35 | 0.21 | 0.18 | 0.25 | 0.25 | 0.2 | 0.2 | 0.28 |
| | 6ug | 18 | 0.25 | 0.3 | 0.25 | 0.2 | 0.28 | 0.22 | 0.29 | 0.23 | 0.34 |
| | 3ug | 7.3 | 0.28 | 0.24 | 0.46 | 0.26 | 0.24 | 0.27 | 0.22 | 0.25 | 0.26 |
| 12 µg | off sum | 42.22 | 1.67 | 1.53 | 3.17 | 1.19 | 1.46 | 1.74 | 1.33 | 2.05 | 1.28 |
| | on/off | 2.3 | 59 | 65 | 31 | 84 | 68 | 57 | 75 | 48 | 0.09 |
| 6 µg | off sum | 19.14 | 5.94 | 1.53 | 1.53 | 1.06 | 1.28 | 1.36 | 1.3 | 1.32 | 1.22 |
| | on/off | 5.1 | 17 | 65 | 65 | 94 | 77 | 73 | 76 | 75 | 0.12 |
| 3 µg | off sum | 8.28 | 4.3 | 1.18 | 1.56 | 1.29 | 1.22 | 1.54 | 1.21 | 8.13 | 1.2 |
| | on/off | 12 | 23 | 83 | 63 | 77 | 81 | 63 | 82 | 12 | 0.13 |
| sum off | | 69.63 | 11.91 | 4.23 | 6.26 | 3.54 | 3.96 | 4.64 | 3.84 | 11.5 | 3.7 |
| Ave. on/off | | 6.3 | 33 | 71 | 53 | 85 | 75 | 65 | 77 | 45 | 0.11 |

TABLE 7b

On Target and Off Target cleavage at Site E

| | SITE E | 55266-2A-53853 | 55266-2A-68815 | 68815-2A-55266 | 55266-2A-68879 | 68879-2A-55266 | 68798-2A-68815 | 68815-2A-68798 | 68846-2A-53853 | 53853-2A-68846 | Site E control |
|---|---|---|---|---|---|---|---|---|---|---|---|
| On Target | 12ug | 96.7 | 97.6 | 86.5 | 96.3 | 95.5 | 97.5 | 96.4 | 96.5 | 97 | 0.19 |
| | 6ug | 95.3 | 94.6 | 81.3 | 95.2 | 91.5 | 96.5 | 97.2 | 94.4 | NA | 0.34 |
| | 3ug | 95.3 | NA | NA | NA | NA | NA | NA | NA | NA | 0.17 |
| Off E1 | 12ug | 1.24 | 0.32 | 0.23 | 0.24 | 0.3 | 0.23 | 0.27 | 0.19 | 0.21 | 0.29 |
| | 6ug | 0.79 | 0.23 | 0.24 | 0.27 | 0.25 | 0.22 | 0.22 | 0.18 | 0.23 | 0.25 |
| | 3ug | 0.5 | 0.26 | 0.18 | 0.2 | 0.23 | 0.2 | 0.23 | 0.23 | 0.23 | 0.26 |
| Off E2 | 12ug | 19.69 | 1.05 | 0.51 | 0.95 | 1.04 | 0.37 | 0.36 | 0.18 | 0.23 | 0.24 |
| | 6ug | 11.09 | NA | 0.34 | 0.67 | 0.69 | 0.31 | 0.26 | 0.17 | 0.22 | 0.17 |
| | 3ug | 4.05 | 0.36 | 0.28 | 0.34 | 0.33 | 0.24 | 0.26 | 0.23 | 0.22 | 0.13 |
| Off E3 | 12ug | 4.32 | 0.14 | 0.19 | 0.4 | 0.19 | 0.17 | 0.19 | 0.18 | 0.16 | 0.19 |
| | 6ug | 1.33 | 0.13 | 0.13 | 0.21 | 0.17 | 0.19 | 0.14 | 0.11 | 0.19 | 0.21 |
| | 3ug | 0.47 | 0.13 | 0.15 | 0.2 | 0.18 | 0.14 | 0.15 | 0.12 | 0.1 | 0.14 |
| 12ug | off sum | 25.24 | 1.51 | 0.93 | 1.59 | 1.53 | 0.77 | 0.82 | 0.54 | 0.6 | 0.71 |
| | on/off | 3.8 | 65 | 93 | 61 | 62 | 127 | 117 | 177 | 161 | 0.27 |
| 6ug | off sum | 13.21 | 0.36 | 0.72 | 1.15 | 1.11 | 0.72 | 0.61 | 0.46 | 0.64 | 0.63 |
| | on/off | 7.2 | 261 | 113 | 83 | 82 | 135 | 160 | 204 | NA | 0.54 |
| 3ug | off sum | 5.02 | 0.74 | 0.61 | 0.74 | 0.74 | 0.57 | 0.64 | 0.58 | 0.55 | 0.52 |
| | on/off | 18.98 | NA | NA | NA | NA | NA | NA | NA | NA | 0.32 |
| sum off | | 43.47 | 2.62 | 2.26 | 3.48 | 3.38 | 2.06 | 2.07 | 1.59 | 1.79 | 1.86 |
| Ave. on/off | | 10 | 163 | 103 | 72 | 72 | 131 | 139 | 191 | 161 | 0.38 |

Thus, following modifications, the ZFN reagents maintained the excellent on-target cutting activity, often while diminishing off-target cleavage activity to background (compare for example, the on-target cleavage activity of the parental 55254/55248 pair with the modified 68861/68796 pair, showing 96.7 and 99.3 percent on target cleavage at the saturating doses of 12 µg, respectively, while also having a total off target activity as this dose of 42.22 percent in the parent pair and 1.19% in the modified pair- similar to the control level of 1.28.

As with the TRAC ZFNs: potential phosphate contacting amino acids were modified in the FokI domain of the B2M proteins. Exemplary modifications of the ZFP components (“designs”) are shown below in Table 8.

TABLE 8

B2M-specific ZFN optimization

| SBS # (target site, 5′-3′) | Design [Helix Sequence, SEQ ID] | | | | | | Linker |
|---|---|---|---|---|---|---|---|
| | [Mutations to finger backbone] | | | | | | Fok mutants |
| | F1 | F2 | F3 | F4 | F5 | F6 | |
| SBS57531 5′ gaGTAGCG cGAGCACA GCtaaggc cacg (SEQ ID NO:126) | AQCCLFH (SEQ ID NO:128) | DQSNLRA (SEQ ID NO:42) | RSANLTR (SEQ ID NO:129) | RSDDLTR (SEQ ID NO:130) | QSGSLTR (SEQ ID NO: 66) | N/A | N6a KKR N-term Fok |
| SBS68957 5′ gaGTAGCG cGAGCACA GCtaaggc cacg (SEQ ID NO:126) | AQCCLFH (SEQ ID NO:128) | DQSNLRA (SEQ ID NO:42) | RSANLTR (SEQ ID NO:129) | RSDDLTR (SEQ ID NO:130) | QSGSLTR (SEQ ID NO: 66) | N/A | N6a |
| | none | none | None | none | none | N/A | KKR K525S N-term Fok |
| SBS72678 5′ gaGTAGCG cGAGCACA GCtaaggc cacg (SEQ ID NO:126) | AQCCLFH (SEQ ID NO:128) | DQSNLRA (SEQ ID NO:42) | RSANLTR (SEQ ID NO:129) | RSDDLTR (SEQ ID NO:130) | QSGSLTR (SEQ ID NO: 66) | N/A | N6a |
| | none | none | None | none | none | N/A | KKR R416Y N-term Fok |
| SBS57071 gcCACGGA gCGAGACA TCTCGgcc cgaa (SEQ ID NO:127) | RSDDLSK (SEQ ID NO: 131) | DSSARKK (SEQ ID NO:132) | DRSNLSR (SEQ ID NO:22) | QRTHLRD (SEQ ID NO:133) | QSGHLAR (SEQ ID NO:29) | DSSNREA (SEQ ID NO: 134) | L0 ELD C-term Fok |
| SBS72732 gcCACGGA gCGAGACA TCTCGgcc cgaa (SEQ ID NO:127) | RSDDLSK (SEQ ID NO: 131) | DSSARKK (SEQ ID NO:132) | DRSNLSR (SEQ ID NO:22) | QRTHLRD (SEQ ID NO:133) | QSGHLAR (SEQ ID NO:29) | DSSNREA (SEQ ID NO: 134) | L0 |
| | none | none | None | none | none | none | ELD P478D C-term Fok |
| SBS72748 gcCACGGA gCGAGACA | RSDDLSK (SEQ ID NO: 131) | DSSARKK (SEQ ID NO:132) | DRSNLSR (SEQ ID NO:22) | QRTHLRD (SEQ ID NO:133) | QSGHLAR (SEQ ID NO:29) | DSSNREA (SEQ ID NO: 134) | L0 |
| TCTCGgcc cgaa (SEQ ID NO:127) | none | none | None | none | none | N/A | ELD Q481D C-term Fok |

The modified B2M reagents were tested for activity as above and were analyzed for phenotypic knockout by FACs analysis using an antibody specific for HLA. All pairwise combinations (57531/57071; 57531/72732; 57531/72748; 68957/57071; 68957/72732; 68957/72748; 72678/57071; 72678/72732; 72678/72748) were found be active with exemplary results for the indicated pairs shown below in Table 9 and demonstrate that the modified variants are active.

TABLE 9

Phenotypic analysis of B2M-specific ZFN

| ZFN pair (2A mRNA) | ZFN Concentration (µg/mL) | | | |
|---|---|---|---|---|
| | 30 | 60 | 90 | 120 |
| | % Indels | | | |
| 57071/68957 | 74 | 79 | 83 | 81 |
| 72732/57531 | 83 | 86 | 87 | 85 |
| 72732/72678 | 86 | nt | nt | 87 |
| 72748/68957 | 37 | nt | nt | 80 |

nt: not tested.

On- and off-target analyses were also carried out using MiSeq for each of the pairs listed above in Table 9. The results are shown below for each pair in tables 10A- 10D, and demonstrate that these reagents are highly specific.

TABLE 10A

Off target analysis for 57071/68957 pair

| | ZFP | | GFP | | | |
|---|---|---|---|---|---|---|
| 57071/68957 | corrected | raw | corrected | raw | p-value | curation |
| Target | 91.64 | 91.94 | 0.19 | 0.25 | 0.00 | positive |
| OT1 | 0.08 | 0.39 | 0.04 | 0.35 | 0.12 | negative |
| OT2 | 0.03 | 0.33 | 0.01 | 0.24 | 0.06 | negative |
| OT3 | 0.08 | 1.22 | 0.03 | 1.00 | 0.05 | negative |
| OT4 | 0.02 | 0.16 | 0.03 | 0.14 | 1.00 | negative |
| OT5 | 0.04 | 0.48 | 0.02 | 0.41 | 1.00 | negative |
| OT6 | 0.04 | 0.27 | 0.03 | 0.22 | 1.00 | maybe |
| OT7 | nt | nt | nt | nt | nt | nt |
| OT8 | 0.02 | 0.18 | 0.02 | 0.13 | 1.00 | negative |
| OT9 | 0.04 | 0.72 | 0.06 | 0.58 | 1.00 | negative |
| OT10 | 0.03 | 0.15 | 0.03 | 0.12 | 1.00 | negative |

TABLE 10B

Off target analysis for 72732/57531 pair

| | ZFP | | GFP | | | |
|---|---|---|---|---|---|---|
| 72732/57531 | corrected | raw | corrected | raw | p-value | curation |
| Target | 95.75 | 96.88 | 0.25 | 0.31 | 0.00 | positive |
| OT1 | 0.03 | 0.26 | 0.02 | 0.26 | 1.00 | negative |
| OT2 | 0.08 | 0.52 | 0.06 | 0.41 | 1.00 | negative |
| OT3 | 0.06 | 0.19 | 0.05 | 0.21 | 1.00 | negative |
| OT4 | 0.06 | 0.47 | 0.04 | 0.40 | 1.00 | negative |
| OT5 | 0.03 | 0.19 | 0.02 | 0.19 | 1.00 | negative |
| OT6 | 0.02 | 0.77 | 0.02 | 0.84 | 1.00 | negative |
| OT7 | 0.04 | 0.98 | 0.06 | 0.79 | 1.00 | negative |
| OT8 | 0.07 | 7.42 | 0.07 | 7.45 | 1.00 | negative |
| OT9 | 0.02 | 0.14 | 0.02 | 0.16 | 1.00 | negative |
| OT10 | 0.03 | 0.27 | 0.03 | 0.28 | 1.00 | negative |

TABLE 10C

Off target analysis for 72732/72678 pair

| | ZFP | | GFP | | | |
|---|---|---|---|---|---|---|
| 72732/72678 | corrected | raw | corrected | raw | p-value | curation |
| Target | 94.76 | 95.23 | 0.17 | 0.21 | 0.00 | positive |
| OT1 | 0.09 | 0.48 | 0.02 | 0.36 | 0.00 | negative |
| OT2 | 0.05 | 0.37 | 0.02 | 0.39 | 0.43 | maybe |
| OT3 | 0.03 | 0.28 | 0.03 | 0.19 | 1.00 | negative |
| OT4 | 0.02 | 0.18 | 0.01 | 0.15 | 1.00 | negative |
| OT5 | 0.01 | 0.09 | 0.03 | 0.11 | 1.00 | negative |
| OT6 | 0.09 | 0.42 | 0.03 | 0.41 | 0.00 | negative |
| OT7 | 1.02 | 17.40 | 2.35 | 19.23 | 1.00 | negative |
| OT8 | 0.07 | 0.71 | 0.04 | 0.58 | 1.00 | negative |
| OT9 | 0.02 | 0.21 | 0.05 | 0.20 | 1.00 | negative |
| OT10 | 0.03 | 0.25 | 0.02 | 0.18 | 1.00 | negative |

TABLE 10D

Off target analysis for 72748/68957 pair

| | ZFP | | GFP | | | |
|---|---|---|---|---|---|---|
| 72748/68957 | corrected | raw | corrected | raw | p-value | curation |
| Target | 93.39 | 93.50 | 0.16 | 0.20 | 0.00 | positive |
| OT1 | 0.05 | 0.30 | 0.02 | 0.24 | 0.69 | negative |
| OT2 | 0.02 | 0.14 | 0.02 | 0.14 | 1.00 | negative |
| OT3 | 0.05 | 2.24 | 0.04 | 2.29 | 1.00 | negative |
| OT4 | 0.02 | 0.33 | 0.03 | 0.31 | 1.00 | negative |
| OT5 | 0.05 | 7.57 | 0.07 | 7.21 | 1.00 | negative |
| OT6 | 0.03 | 1.03 | 0.03 | 1.03 | 1.00 | negative |
| OT7 | 0.76 | 1.86 | 0.59 | 1.79 | 1.00 | negative |
| OT8 | 0.02 | 0.14 | 0.02 | 0.13 | 1.00 | negative |
| OT9 | 0.03 | 0.23 | 0.03 | 0.29 | 1.00 | negative |
| OT10 | 0.33 | 94.52 | 0.29 | 94.49 | 1.00 | negative |

The modified TRAC- and B2M- specific ZFNs were tested in combination and evaluated for knock out efficiency, both by Miseq analysis and by phenotypic analysis analyzing the amount of CD3+ or HLA+ cells by FACs analysis. The analysis was done in T cells, using two different concentrations of added ZFN-encoding mRNA (90 µg/mL or 120 µg/mL). The results are shown below in Table 11 and demonstrate that these reagents are highly efficient.

TABLE 11

TRAC/B2M cleavage

| ZFN reagents (2A-mRNAs) | | Phenotypic screen | | Miseq analysis | |
|---|---|---|---|---|---|
| 68846-2A-53853 (TRAC) µg/mL | 72732-2A-72678 (B2M) µg/mL | %CD3-neg | %HLA-I-neg | %TRAC indels | %B2M indels |
| 0 | 30 | - | 86 | - | 95 |
| 60 | 0 | 98 | - | 92 | - |
| 90 | 90 | 95 | 86 | 90 | 95 |
| 120 | 90 | 94 | 86 | 90 | 94 |
| 90 | 120 | 94 | 86 | 90 | 95 |
| 120 | 120 | 95 | 87 | 91 | 95 |

The reagents were also tested in combination in the presence or absence of a GFP donor construct driven by a PGK promoter. The results are shown in Table 12 where the insertion was done either into the cleaved B2M or TRAC locus. In each case, the PGK-GFP donor was delivered by AAV6 and comprised homology arms with homology flanking either the TRAC or B2M cut sites. The TRAC-specific ZFN pair construct used was 68846-2A-53853 while the construct for the B2M specific pair was 72732-2A-72678.

TABLE 12

Activity of double knock out in two T cell donors.

| T cell donor #1 | | | T cell donor #2 | | |
|---|---|---|---|---|---|
| Sample | Targeted locus | % indel | Sample | Targeted locus | % indel |
| Mock | B2M | 0.3 | Mock | B2M | 0.04 |
| TRAC + B2M | B2M | 84.14 | TRAC + B2M | B2M | 75.33 |
| TRAC + B2M PGK-GFP | B2M | 83.55 | TRAC + B2M PGK-GFP | B2M | 80.96 |
| Mock | TRAC | 0.08 | Mock | TRAC | 0.38 |
| TRAC + B2M | TRAC | 88.05 | TRAC + B2M | TRAC | 85.09 |
| TRAC + B2M PGK-GFP | TRAC | 78.94 | TRAC + B2M PGK-GFP | TRAC | 74.54 |

Thus, optimized pairs of ZFNs specific for B2M were constructed by choosing a FokI variant (see above) in combination with a ZFP DNA binding domain.

The optimized amino acid sequences for the DNA binding domain for the B2M ZFNs 72732 and 72678 are shown below:

72732 N term:

RPFQCRICMRNFSRSDDLSKHIRTHTGEKPFACDICGRKFADSSARKKHT
KIHTGEKPFQCRICMRNFSDRSNLSRHIRTHTGEKPFACDICGRKFAQRT
HLRDHTKIHTHPRAPIPKPFQCRICMRNFSQSGHLARHIRTHTGEKPFAC
DICGRKFADSSNREAHTKIH (SEQ ID NO: 175)

72678 C-term:

RPFQCRICMRKFAAQCCLFHHTKIHTGEKPFQCRICMRNFSDQSNLRAHI
RTHTGEKPFACDICGRKFARSANLTRHTKIHTHPRAPIPKPFQCRICMRN
FSRSDDLTRHIRTHTGEKPFACDICGRKFAQSGSLTRHTKIH (SEQ ID
NO: 176)

Additional ZFNs comprising the modified ZFPs of the ZFNs described herein (e.g., SEQ ID NO: 175 and SEQ ID NO: 176) are also generated using different FokI and/or linker domains.

Similarly, the optimized pairs of ZFNs specific for TRAC were constructed by choosing a FokI variant (see for example above) in combination with a ZFP DNA binding domain. The optimized amino acid sequences for the DNA binding domain for the B2M ZFNs 68846 and 53853 are shown below:

68846 C-term:

RPFQCRICMQNFSQSSDLSRHIRTHTGEKPFACDICGRKFAQSGNRTTHT
KIHTHPRAPIPKPFQCRICMQNFSRSANLARHIRTHTGEKPFACDICGRK
FADRSALARHTKIHTGSQKPFQCRICMQNFSRSDVLSEHIRTHTGEKPFA
CDICGRKFAKHSTRRVHTKIH (SEQ ID NO: 177)

53853 N-term:

RPFQCRICMRNFSTMHQRVEHIRTHTGEKPFACDICGRKFATSGHLSRHT
KIHTGSQKPFQCRICMRNFSRSDHLTQHIRTHTGEKPFACDICGRKFADS
ANLSRHTKIHTHPRAPIPKPFQCRICMRNFSQSGSLTRHIRTHTGEKPFA
CDICGRKFAAKWNLDAHTKIH SEQ ID NO: 178).

The ZFNs may be assembled with the DNA binding domain N terminal to the FokI domain, wherein the linker sequence between the DNA binding domain and the FokI domain was the L0 linker: LRGS (SEQ ID NO: 190). Alternatively, if the ZFN is assembled such that the FokI domain is N-terminal to the DNA binding domain, the linker used was the N7c linker: SGAIRCHDEFWF (SEQ ID NO: 179).

Additional features were added into the constructs including a 3x FLAG TAG in the N-terminus region (DYKDHDGDYKDHDIDYKDDDDK, SEQ ID NO: 180), and a nuclear localization sequence (PKKKRKV, SEQ ID NO: 181).

In addition, in some constructs, sequences encoding the ZFN pair of interest are linked together in one DNA sequence where the open reading frames for each ZFN partner are separated by a 2A sequence. Such a DNA sequence, for the 68846-2A-53853 is shown below:

5′ATGGACTACAAAGACCATGACGGTGATTATAAAGATCATGACATCGAT
TACAAGGATGACGATGACAAGATGGCCCCCAAGAAGAAGAGGAAGGTCGG
CATCCACGGGGTACCCGCCGCTATGGGACAGCTGGTGAAGAGCGAGCTGG
AGGAGAAGAAGTCCGAGCTGCGGCACAAGCTGAAGTACGTGCCCCACGAG
TACATCGAGCTGATCGAGATCGCCAGGAACAGCACCCAGGACCGCATCCT
GGAGATGAAGGTGATGGAGTTCTTCATGAAGGTGTACGGCTACAGGGGAA
AGCACCTGGGCGGAAGCAGAAAGCCTGACGGCGCCATCTATACAGTGGGC
AGCCCCATCGATTACGGCGTGATCGTGGACACAAAGGCCTACAGCGGCGG
CTACAATCTGCCTACCGGCCAGGCCGACGAGATGGAGAGATACGTGGAGG
AGAACCAGACCCGGGATAAGCACCTCAACCCCAACGAGTGGTGGAAGGTG
TACCCTAGCAGCGTGACCGAGTTCAAGTTCCTGTTCGTGAGCGGCCACTT
CAAGGGCAACTACAAGGCCCAGCTGACCAGGCTGAACCACATCACCAACT
GCAATGGCGCCGTGCTGAGCGTGGAGGAGCTGCTGATCGGCGGCGAGATG
ATCAAAGCCGGCACCCTGACACTGGAGGAGGTGCGGCGCAAGTTCAACAA
CGGCGAGATCAACTTCAGCGGCGCCATCAGATGCCACGACGAGTTCTGGT
TCAGGCCCTTCCAGTGTCGAATCTGCATGCAGAACTTCAGTCAGTCCTCC
GACCTGTCCCGCCACATCCGCACCCACACCGGCGAGAAGCCTTTTGCCTG
TGACATTTGTGGGAGGAAATTTGCCCAGTCCGGCAACCGCACCACCCATA
CCAAGATACACACGCATCCCAGGGCACCTATTCCCAAGCCCTTCCAGTGT
CGAATCTGCATGCAGAACTTCAGTCGCTCCGCCAACCTGGCCCGCCACAT
CCGCACCCACACCGGCGAGAAGCCTTTTGCCTGTGACATTTGTGGGAGGA
AATTTGCCGACCGCTCCGCCCTGGCCCGCCATACCAAGATACACACGGGA
TCTCAGAAGCCCTTCCAGTGTCGAATCTGCATGCAGAACTTCAGTCGCTC
CGACGTGCTGTCCGAGCACATCCGCACCCACACCGGCGAGAAGCCTTTTG
CCTGTGACATTTGTGGGAGGAAATTTGCCAAGCACTCCACCCGCCGCGTG
CATACCAAGATACACCTGCGGCAGAAGGACAGATCTGGCGGCGGAGAGGG
CAGAGGAAGTCTTCTAACCTGCGGTGACGTGGAGGAGAATCCCGGCCCTA
GGACCATGGACTACAAAGACCATGACGGTGATTATAAAGATCATGACATC
GATTACAAGGATGACGATGACAAGATGGCCCCCAAGAAGAAGAGGAAGGT
CGGCATTCATGGGGTACCCGCCGCTATGGCTGAGAGGCCCTTCCAGTGTC
GAATCTGCATGCGTAACTTCAGTACCATGCACCAGCGCGTGGAGCACATC
CGCACCCACACCGGCGAGAAGCCTTTCGCCTGTGACATTTGTGGGAGGAA
ATTTGCCACCTCCGGCCACCTGTCCCGCCATACCAAGATACACACGGGCA
GCCAAAAGCCCTTCCAGTGTCGAATCTGCATGCGTAACTTCAGTCGCTCC
GACCACCTGACCCAGCACATCCGCACCCACACCGGCGAGAAGCCTTTTGC
CTGTGACATTTGTGGGAGGAAATTTGCCGACTCCGCCAACCTGTCCCGCC
ATACCAAGATACACACGCACCCGCGCGCCCCGATCCCGAAGCCCTTCCAG
TGTCGAATCTGCATGCGTAACTTCAGTCAGTCCGGCTCCCTGACCCGCCA
CATCCGCACCCACACCGGCGAGAAGCCTTTTGCCTGTGACATTTGTGGGA
GGAAATTTGCCGCCAAGTGGAACCTGGACGCCCATACCAAGATACACCTG
CGGGGATCCCAGCTGGTGAAGAGCGAGCTGGAGGAGAAGAAGTCCGAGCT
GCGGCACAAGCTGAAGTACGTGCCCCACGAGTACATCGAGCTGATCGAGA
TCGCCAGGAACAGCACCCAGGACCGCATCCTGGAGATGAAGGTGATGGAG
TTCTTCATGAAGGTGTACGGCTACAGGGGAAAGCACCTGGGCGGAAGCAG
AAAGCCTGACGGCGCCATCTATACAGTGGGCAGCCCCATCGATTACGGCG
TGATCGTGGACACAAAGGCCTACAGCGGCGGCTACAATCTGCCTATCGGC
CAGGCCGACGAGATGCAGAGATACGTGAAGGAGAACCAGACCCGGAATAA
GCACATCAACCCCAACGAGTGGTGGAAGGTGTACCCTAGCAGCGTGACCG
AGTTCAAGTTCCTGTTCGTGAGCGGCCACTTCAAGGGCAACTACAAGGCC
CAGCTGACCAGGCTGAACCGCAAAACCAACTGCAATGGCGCCGTGCTGAG
CGTGGAGGAGCTGCTGATCGGCGGCGAGATGATCAAAGCCGGCACCCTGA
CACTGGAGGAGGTGCGGCGCAAGTTCAACAACGGCGAGATCAACTTCTGA
TAA(SEQ ID NO: 182) .

The amino acid sequence of the 68846-2A-53853 open reading frame is:

• MDYKDHDGDY KDHDIDYKDD DDKMAPKKKR KVGIHGVPAA MGQLVKSELE EKKSELRHKL 1-60
KYVPHEYIEL IEIARNSTQD RILEMKVMEF FMKVYGYRGK HLGGSRKPDG AIYTVGSPID 61-120
YGVIVDTKAY SGGYNLPTGQ ADEMERYVEE NQTRDKHLNP NEWWKVYPSS VTEFKFLFVS 121-180
GHFKGNYKAQ LTRLNHITNC NGAVLSVEEL LIGGEMIKAG TLTLEEVRRK FNNGEINFSG 181-240
AIRCHDEFWF RPFQCRICMQ NFSQSSDLSR HIRTHTGEKP FACDICGRKF AQSGNRTTHT 241-300
KIHTHPRAPI PKPFQCRICM QNFSRSANLA RHIRTHTGEK PFACDICGRK FADRSALARH 301-360
TKIHTGSQKP FQCRICMQNF SRSDVLSEHI RTHTGEKPFA CDICGRKFAK HSTRRVHTKI 361-420
HLRQKDRSGG GEGRGSLLTC GDVEENPGPR TMDYKDHDGD YKDHDIDYKD DDDKMAPKKK 421-480
RKVGIHGVPA AMAERPFQCR ICMRNFSTMH QRVEHIRTHT GEKPFACDIC GRKFATSGHL 481-540
SRHTKIHTGS QKPFQCRICM RNFSRSDHLT QHIRTHTGEK PFACDICGRK FADSANLSRH 541-600
TKIHTHPRAP IPKPFQCRIC MRNFSQSGSL TRHIRTHTGE KPFACDICGR KFAAKWNLDA 601-660
HTKIHLRGSQ LVKSELEEKK SELRHKLKYV PHEYIELIEI ARNSTQDRIL EMKVMEFFMK 661-720
VYGYRGKHLG GSRKPDGAIY TVGSPIDYGV IVDTKAYSGG YNLPIGQADE MQRYVKENQT 721-780
RNKHINPNEW WKVYPSSVTE FKFLFVSGHF KGNYKAQLTR LNRKTNCNGA VLSVEELLIG 781-840
GEMIKAGTLT LEEVRRKFNN GEINF (SEQ ID NO:183) 841-
865

The features of this polypeptide are broken out below in Table 13.

TABLE 13

Features of 68846-2A-53853 peptide sequence

| Feature | Designation | Location (within SEQ ID NO:183) |
|---|---|---|
| 3xFLAG sequence | xx | 2-23 |
| Nuclear localization sequence | xx | 26-32 |
| ELD I479T Fok1 domain | xx | 43-238 |
| N7c linker | xx | 239-250 |
| 68846 DNA binding domain | xx | 251-421 |
| 2A Linker | xx | 432-449 |
| 3xFLAG sequence | xx | 452-474 |
| Nuclear localization sequence | xx | 477-483 |
| 53853 DNA binding domain | xx | 495- 665 |
| L0 linker | xx | 666-669 |
| KKR FokI domain | xx | 670-865 |

The sequence for the 72732-2A-72678 opening reading frame is shown below:

• ATGGACTACA AAGACCATGA CGGTGATTAT AAAGATCATG ACATCGATTA CAAGGATGAC
GATGACAAGA TGGCCCCCAA GAAGAAGAGG AAGGTCGGCA TCCACGGGGT ACCCGCCGCT
ATGGCTGAGA GGCCCTTCCA GTGTCGAATC TGCATGCGTA ACTTCAGTCG TAGTGACGAC
CTGAGCAAGC ACATCCGCAC CCACACAGGC GAGAAGCCTT TTGCCTGTGA CATTTGTGGG
AGGAAATTTG CCGACAGCAG CGCCCGCAAA AAGCATACCA AGATACACAC GGGCGAGAAG
CCCTTCCAGT GTCGAATCTG CATGCGTAAC TTCAGTGACC GCTCCAACCT GTCCCGCCAC
ATCCGCACCC ACACCGGCGA GAAGCCTTTT GCCTGTGACA TTTGTGGGAG GAAATTTGCC
CAGCGCACCC ACCTGCGCGA CCATACCAAG ATACACACGC ACCCGCGCGC CCCGATCCCG
AAGCCCTTCC AGTGTCGAAT CTGCATGCGT AACTTCAGTC AGTCCGGCCA CCTGGCCCGC
CACATCCGCA CCCACACCGG CGAGAAGCCT TTTGCCTGTG ACATTTGTGG GAGGAAATTT
GCCGACTCCT CCAACCGCGA GGCCCATACC AAGATACACC TGCGGGGATC CCAGCTGGTG
AAGAGCGAGC TGGAGGAGAA GAAGTCCGAG CTGCGGCACA AGCTGAAGTA CGTGCCCCAC
GAGTACATCG AGCTGATCGA GATCGCCAGG AACAGCACCC AGGACCGCAT CCTGGAGATG
AAGGTGATGG AGTTCTTCAT GAAGGTGTAC GGCTACAGGG GAAAGCACCT GGGCGGAAGC
AGAAAGCCTG ACGGCGCCAT CTATACAGTG GGCAGCCCCA TCGATTACGG CGTGATCGTG
GACACAAAGG CCTACAGCGG CGGCTACAAT CTGGACATCG GCCAGGCCGA CGAGATGGAG
AGATACGTGG AGGAGAACCA GACCCGGGAT AAGCACCTCA ACCCCAACGA GTGGTGGAAG
GTGTACCCTA GCAGCGTGAC CGAGTTCAAG TTCCTGTTCG TGAGCGGCCA CTTCAAGGGC
AACTACAAGG CCCAGCTGAC CAGGCTGAAC CACATCACCA ACTGCAATGG CGCCGTGCTG
AGCGTGGAGG AGCTGCTGAT CGGCGGCGAG ATGATCAAAG CCGGCACCCT GACACTGGAG
GAGGTGCGGC GCAAGTTCAA CAACGGCGAG ATCAACTTCA GATCTGGCGG CGGAGAGGGC
AGAGGAAGTC TTCTAACCTG CGGTGACGTG GAGGAGAATC CCGGCCCTAG GACCATGGAC
TACAAAGACC ATGACGGTGA TTATAAAGAT CATGACATCG ATTACAAGGA TGACGATGAC
AAGATGGCCC CCAAGAAGAA GAGGAAGGTC GGCATTCATG GGGTACCCGC CGCTATGGGA
CAGCTGGTGA AGAGCGAGCT GGAGGAGAAG AAGTCCGAGC TGCGGCACAA GCTGAAGTAC
GTGCCCCACG AGTACATCGA GCTGATCGAG ATCGCCTACA ACAGCACCCA GGACCGCATC
CTGGAGATGA AGGTGATGGA GTTCTTCATG AAGGTGTACG GCTACAGGGG AAAGCACCTG
GGCGGAAGCA GAAAGCCTGA CGGCGCCATC TATACAGTGG GCAGCCCCAT CGATTACGGC
GTGATCGTGG ACACAAAGGC CTACAGCGGC GGCTACAATC TGCCTATCGG CCAGGCCGAC
GAGATGCAGA GATACGTGAA GGAGAACCAG ACCCGGAATA AGCACATCAA CCCCAACGAG
TGGTGGAAGG TGTACCCTAG CAGCGTGACC GAGTTCAAGT TCCTGTTCGT GAGCGGCCAC
TTCAAGGGCA ACTACAAGGC CCAGCTGACC AGGCTGAACC GCAAAACCAA CTGCAATGGC
GCCGTGCTGA GCGTGGAGGA GCTGCTGATC GGCGGCGAGA TGATCAAAGC CGGCACCCTG
ACACTGGAGG AGGTGCGGCG CAAGTTCAAC AACGGCGAGA TCAACTTCAG CGGCGCTCAG
GGATCTACCC TGGACTTTAG GCCCTTCCAG TGTCGAATCT GCATGCGTAA GTTTGCCGCC
CAGTGTTGTC TGTTCCACCA TACCAAGATA CACACGGGCG AGAAGCCCTT CCAGTGTCGA
ATCTGCATGC GTAACTTCAG TGACCAGTCC AACCTGCGCG CCCACATCCG CACCCACACC
GGCGAGAAGC CTTTTGCCTG TGACATTTGT GGGAGGAAAT TTGCCCGCTC CGCCAACCTG
ACCCGCCATA CCAAGATACA CACGCACCCG CGCGCCCCGA TCCCGAAGCC CTTCCAGTGT
CGAATCTGCA TGCGTAACTT CAGTCGCTCC GACGACCTGA CCCGCCACAT CCGCACCCAC
ACCGGCGAGA AGCCTTTTGC CTGTGACATT TGTGGGAGGA AATTTGCCCA GTCCGGCTCC
CTGACCCGCC ATACCAAGAT ACACCTGCGG CAGAAGGACT GATAA (SEQ ID NO:184)

The amino acid sequence of the 72732-2A-72678 open reading is shown below.

MDYKDHDGDY KDHDIDYKDD DDKMAPKKKR KVGIHGVPAA MAERPFQCRI CMRNFSRSDD 1-60
LSKHIRTHTG EKPFACDICG RKFADSSARK KHTKIHTGEK PFQCRICMRN FSDRSNLSRH 61-120
IRTHTGEKPF ACDICGRKFA QRTHLRDHTK IHTHPRAPIP KPFQCRICMR NFSQSGHLAR 121-180
HIRTHTGEKP FACDICGRKF ADSSNREAHT KIHLRGSQLV KSELEEKKSE LRHKLKYVPH 181-240
EYIELIEIAR NSTQDRILEM KVMEFFMKVY GYRGKHLGGS RKPDGAIYTV GSPIDYGVIV 241-300
DTKAYSGGYN LDIGQADEME RYVEENQTRD KHLNPNEWWK VYPSSVTEFK FLFVSGHFKG 301-360
NYKAQLTRLN HITNCNGAVL SVEELLIGGE MIKAGTLTLE EVRRKFNNGE INFRSGGGEG 361-420
RGSLLTCGDV EENPGPRTMD YKDHDGDYKD HDIDYKDDDD KMAPKKKRKV GIHGVPAAMG 421-480
QLVKSELEEK KSELRHKLKY VPHEYIELIE IAYNSTQDRI LEMKVMEFFM KVYGYRGKHL 481-540
GGSRKPDGAI YTVGSPIDYG VIVDTKAYSG GYNLPIGQAD EMQRYVKENQ TRNKHINPNE 541-600
WWKVYPSSVT EFKFLFVSGH FKGNYKAQLT RLNRKTNCNG AVLSVEELLI GGEMIKAGTL 601-660
TLEEVRRKFN NGEINFSGAQ GSTLDFRPFQ CRICMRKFAA QCCLFHHTKI HTGEKPFQCR 661-720
ICMRNFSDQS NLRAHIRTHT GEKPFACDIC GRKFARSANL TRHTKIHTHP RAPIPKPFQC 721-780
RICMRNFSRS DDLTRHIRTH TGEKPFACDI CGRKFAQSGS LTRHTKIHLR QKD 781-833
(SEQ ID NO:185)

The features of the 72732-2A-72678 amino acid sequence are shown below in Table 14.

TABLE 14

Features of the 72732-2A-72678 amino acid sequence

| Feature | Designation | Location (within SEQ ID NO:185) |
|---|---|---|
| 3x FLAG sequence | xx | 2-23 |
| Nuclear localization sequence | xx | 26-32 |
| 72732 DNA binding domain | xx | 44-213 |
| L0 linker | xx | 214-217 |
| ELD P478D FokI domain | xx | 218-413 |
| 2A Linker | xx | 419-436 |
| 3xFLAG sequence | xx | 440-461 |
| Nuclear localization sequence | xx | 464-470 |
| KKR R416Y FokI domain | xx | 481-676 |
| N6alinker | xx | 677-686 |
| 72678 DNA binding domain | xx | 687-828 |

### Example 6: In Vivo Testing of ZFN Reagents

T cells as described herein are administered to animal models of graft vs. host disease and/or cancer (e.g., nude mice injected with cancer cell lines such as multiple myeloma to establish tumor models). For example, activated human T cells are electroporated with mRNAs encoding the B2M- and TRAC-specific ZFNs where each pair is encoded by a single mRNA separated by a sequence encoding a 2A self-cleaving peptide (MacLeod, et al. (2017) Mol Ther. 25(4):949-961). The cells are also transduced with AAV particles comprising a CAR donor (e.g., CD19 CAR). The cells are then cultured and stained for CAR expression and a lack of CD3+ cells. Any residual CD3+ cells are depleted by magnetic separation. NSG mice are injected intravenously with firefly luciferase expressing Raji cells (Raji-ffLuc) and, after four days, are injected with the CD3-/anti-CD19 CAR T cells. Engraftment and growth of the Raji-ffLuc cells is evident by day four post injection and increases significantly in untreated mice. Peak CAR T cell frequencies in the blood of treated mice are observed on day 8, reaching ~10% of cells in peripheral blood in the high-dose group. By days 17-19, all mice in control groups show evidence of significant tumor burden, especially in the spine and bone marrow, resulting in complete hindlimb paralysis, and are euthanized. In contrast, all groups of mice treated with anti-CD19 CAR T cells show no evidence of tumor growth by day 11 and, remained tumor-free through day 32 of the study.

No or minimal residual disease is detected in tissue of animals (e.g., bone marrow, spleen, lungs, liver, heart, etc.) receiving T cells as described herein. By contrast, control subjects have detectable tumor cells in most tissues.

All patents, patent applications and publications mentioned herein are hereby incorporated by reference in their entirety.

Although disclosure has been provided in some detail by way of illustration and example for the purposes of clarity of understanding, it will be apparent to those skilled in the art that various changes and modifications can be practiced without departing from the spirit or scope of the disclosure. Accordingly, the foregoing description and examples should not be construed as limiting.

What is claimed is:

1. A polynucleotide encoding a zinc finger nuclease comprising:
a ZFP from a ZFN designated 68957, 72678, 72732 or 72748;
an engineered FokI cleavage domain; and
a linker between the FokI cleavage domain and the ZFP.

2. The polynucleotide of claim 1, comprising a 2A sequence.

3. A pharmaceutical composition comprising the polynucleotide of claim 1.

4. A method of modifying an endogenous beta-2-microglobulin (B2M) gene in a cell, the method comprising administering the polynucleotide of claim 1 to the cell such that the endogenous B2M gene is modified.

5. The method of claim 4, further comprising introducing an exogenous sequence into the cell such that the exogenous sequence is inserted into the endogenous B2M gene.

6. The method of claim 4, wherein the modification comprises a deletion.

7. A method of producing a genetically modified cell comprising a genomic modification within an endogenous B2M gene, the method comprising the steps of:
a) contacting a cell with the polynucleotide of claim 1;
b) subjecting the cell to conditions conducive to expressing the fusion protein from the polynucleotide; and
c) modifying the endogenous B2M gene with the expressed fusion protein sufficient to produce the genetically modified cell.

8. A kit comprising the polynucleotide of claim 1.

9. A polynucleotide encoding a zinc finger nuclease (ZFN) comprising left and right ZFNs as follows: a ZFN designated 68796 and a ZFN designated 68813; a ZFN designated 68796 and a ZFN designated 68861; a ZFN designated 68812 and a ZFN designated 68813; a ZFN designated 68876 and a ZFN designated 68877; a ZFN designated 68815 and a ZFN designated 55266; a ZFN designated 68879 and a ZFN designated 55266; a ZFN designated 68798 and a ZFN designated 68815; or a ZFN designated 68846 and a ZFN designated 53853.

10. The polynucleotide of claim 9, wherein the polynucleotide is mRNA.

11. The polynucleotide of claim 9, comprising a 2A sequence between the sequences encoding the left and right ZFNs.

12. A pharmaceutical composition comprising the polynucleotide of claim 9.

13. A method of modifying an endogenous T cell receptor (TCR) gene in a cell, the method comprising administering the polynucleotide of claim 9 to the cell such that the endogenous TCR gene is modified.

14. The method of claim 13, further comprising introducing an exogenous sequence into the cell such that the exogenous sequence is inserted into the endogenous TCR gene.

15. The method of claim 13, wherein the modification comprises a deletion.

16. A method of producing a genetically modified cell comprising a genomic modification within an endogenous TCR gene, the method comprising the steps of:
a) contacting a cell with the polynucleotide of claim 9;
b) subjecting the cell to conditions conducive to expressing the fusion protein from the polynucleotide; and
c) modifying the endogenous TCR gene with the expressed fusion protein sufficient to produce the genetically modified cell.

17. A kit comprising the polynucleotide of claim 9.
